(19) United States
(12) Patent Application Publication
(10) Pub. No.: US 2025/0000881 A1
(43) Pub. Date: Jan. 2, 2025

(54) TREATMENT OF TREATMENT RESISTANT DEPRESSION WITH PSILOCYBIN

(71) Applicant: COMPASS PATHFINDER LIMITED, Altrincham, Cheshire (GB)

(72) Inventors: Trevor Anthony MILL, Oxfordshire (GB); Guy Manning GOODWIN, Oxford (GB); Joyce TSAI, NEW YORK, NY (US); Lindsey MARWOOD, Altrincham (GB); Sunil MISTRY, Watford (GB)

(73) Assignee: COMPASS PATHFINDER LIMITED, Altrincham, Cheshire (GB)

(21) Appl. No.: 18/703,950

(22) PCT Filed: Nov. 2, 2022
(86) PCT No.: PCT/US2022/048713

§ 371 Date: Apr. 23, 2024

Related U.S. Application Data
(60) Provisional application No. 63/277,407, filed on Nov. 9, 2021.
(60) Provisional application No. 63/284,973, filed on Dec. 1, 2021.

(51) Int. Cl. A61K 31/675; A61K 9/48; A61P 25/24

(52) U.S. Cl. CPC A61K 31/675; A61K 9/4858; A61K 9/4866; A61P 25/24

(57) ABSTRACT

The disclosure provides methods for treating treatment-resistant depression a subject in need thereof comprising administering to the subject two or more doses of psilocybin. The methods described herein may be used to treat subjects both responsive and unresponsive to a first dose of psilocybin

### CROSS-REFERENCE TO RELATED APPLICATION

The present application claims the benefit of U.S. Provisional Application 63/277,407 filed Nov. 9, 2021, and 63/284,973 filed on Dec. 1, 2021, the contents of which are hereby incorporated by reference in their entireties.

### BACKGROUND

Depression is one of the most common mental illnesses, affecting more than 264 million people worldwide. It is characterized by depressed mood and markedly diminished interest or pleasure in activities. Other symptoms include significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts.

Current treatments for depression often consist of a combination of psychotherapy and one or more daily medications that regulate neurotransmitters such as dopamine, serotonin, and norepinephrine. These medications often take weeks to months to achieve their full effects and in the meantime, individuals continue to suffer from their symptoms and be at risk of self-harm, as well as harm to their personal and professional lives.

There remains a need in the art for an effective treatment for depression that provides a rapid onset of antidepressant effects within hours or a few days and is sustained long-term.

### SUMMARY

The present disclosure provides methods for treating depression (e.g., treatment-resistant depression) in a subject in need thereof with psilocybin. In some embodiments, the methods relate to the timing of administering a second dose of psilocybin. More specifically, Applicant discovered that the timing for second dose of psilocybin depends on whether the subject responded to the first dose of psilocybin (referred as a “responder”) or did not respond to the first dose of psilocybin (referred as a “non-responder”). For example, in the long term follow-up study (described in Example 3), Applicant discovered that subjects who responded to a first dose of psilocybin experienced a median time to a depressive event of about 189 days (24 days, 95% CI) after administering of a first dose of psilocybin. In some embodiments, the methods provide for administering a second dose of psilocybin before the median time to the depressive event. In some embodiments, Applicant discovered that subjects who did not respond to a first dose of psilocybin should be administered a second dose about 3 weeks or less after being identified as a non-responder.

In some embodiments, the methods described herein comprise treating treatment-resistant depression with psilocybin in a subject that did not respond to a first dose of psilocybin, comprising administering a second dose of psilocybin about 3 weeks after administering the first dose of psilocybin.

In some embodiments, the methods described herein comprise treating treatment-resistant depression in a subject in need thereof, comprising administering a first dose of psilocybin to the subject; measuring the subject's depressive symptoms using a clinical depression evaluation after the first dose of psilocybin; identifying the subject as a non-responder to the first dose of psilocybin; and administering a second dose of psilocybin to the subject 3 weeks after administering the first dose.

In some embodiments, the methods described herein comprise treating treatment-resistant depression in a subject that responded to a first dose of psilocybin, comprising administering a second dose at least about 26 weeks after administering the first dose.

### BRIEF DESCRIPTION OF THE DRAWINGS

FIG. 1 is a numbered structural formula of psilocybin.

FIG. 2A is a XRPD diffractogram of Polymorph A (GM764B).

FIG. 2B is a XRPD diffractogram of Polymorph A′ (JCCA2160F).

FIG. 2C is a XRPD diffractogram of Polymorph B (JCCA2160-F-TM2).

FIG. 2D is a XRPD diffractogram of a Hydrate A (JCCA2157E).

FIG. 2E is a XRPD diffractogram of an ethanol solvate (JCCA2158D).

FIG. 2F is a XRPD diffractogram of product obtained during development of the process (CB646-E) (top)—compared to the diffractograms Polymorph A′ (JCCA2160F) (middle) and Polymorph B (JCCA2160-TM2) (bottom).

FIG. 3A is a DSC and TGA thermograph of Polymorph A (GM764B).

FIG. 3B is a DSC and TGA thermograph of Polymorph A′ (JCCA2160F).

FIG. 3C is a DSC thermograph of Polymorph B (GM748A).

FIG. 3D is a DSC and TGA thermograph of Hydrate A (JCCA2157E).

FIG. 3E is a DSC and TGA thermograph of ethanol solvate (JCCA2158D).

FIG. 4 is a form phase diagram showing the inter-relationship of form in water-based systems.

FIG. 5 is a 1H NMR (Nuclear Magnetic Resonance) spectrum of psilocybin.

FIG. 6 is a 13C NMR spectrum of psilocybin.

FIG. 7 is a FT-IR Spectrum of psilocybin.

FIG. 8 is a Mass Spectrum of psilocybin.

FIG. 9A shows the study design of the clinical trial examining psilocybin in treatment resistant depression in Example 2.

FIG. 9B shows the patient disposition from the clinical trial examining psilocybin in treatment resistant depression in Example 2.

FIG. 9C shows the change from baseline in Montgomery-Åsberg Depression Rating Scale (MADRS) total score observed during the treatment resistant depression study described in Example 2.

FIG. 9D shows the change from baseline in MADRS total score with 95% confidence interval bars observed during the treatment resistant depression study described in Example 2.

FIG. 9E shows the number of MADRS responders by visit observed during the treatment resistant depression study described in Example 2.

FIG. 9F shows the number of MADRS remitters by visit observed during the treatment resistant depression study described in Example 2.

FIG. 9G shows the number of sustained responders at week 12 of the treatment resistant depression study described in Example 2.

### DETAILED DESCRIPTION

### Definitions

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

Furthermore, the term “about” as used herein when referring to a measurable value such as a dose, time, temperature, and the like, is meant to encompass variations acceptable in the art, such as ±20%, ±10%, ±5%, ±1%, ±0.5%, or even ±0.1% of the specified amount. In some embodiments, “about” encompasses variations of ±10% of the specified amount.

Unless the context indicates otherwise, it is specifically intended that the various features described herein can be used in any combination.

As used herein, the terms “reduce,” “decrease,” “lessen” and similar terms mean a decrease of at least about 10%, about 15%, about 20%, about 25%, about 35%, about 50%, about 75%, about 80%, about 85%, about 90%, about 95%, about 97%, or more.

As used herein, the terms “improve,” “increase,” “enhance,” and similar terms indicate an increase of at least about 10%, about 15%, about 20%, about 25%, about 50%, about 75%, about 100%, about 150%, about 200%, about 300%, about 400%, about 500%, or more.

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

As used herein, “substantially absent” with reference to XRPD diffractogram peak means the peak has a relative intensity compared to a reference peak present in the diffractogram of less than about 5%, less than about 4%, less than about 3%, less than about 2%, or less than about 1% of the intensity of the reference peak, or that the peak is not detectable.

XRPD diffractograms and XRPD peak positions may be acquired using Cu Kα radiation.

DSC thermograms and TGA thermograms may be acquired using a heating rate of 20° C./min.

As used herein, the term “diffusion tensor imaging” or “DTI” refers to a technique that detects how water travels along the white matter tracts in the brain. In some embodiments, DTI is used to characterize microstructural changes associated with mental disorders (e.g., major depressive disorder) and/or the response to treatment in subjects with mental disorders.

All disease and disorders listed herein are defined as described in the Diagnostic and Statistical Manual of Mental Disorders (DSM-5), published by the American Psychiatric Association, or in International Classification of Diseases (ICD), published by the World Health Organization. As used herein the term “subject” and “patient” are used interchangeably.

As used herein, “treating” and like terms refer to reducing the severity and/or frequency of one or more symptoms, eliminating one or more symptoms and/or the underlying cause of said symptoms, reducing the frequency or likelihood of one or more symptoms and/or their underlying cause, delaying, preventing and/or slowing the progression of diseases and/or disorders and improving or remediating damage caused, directly or indirectly, by the diseases and/or disorders.

As used herein, “therapeutically-effective dose” means a dose sufficient to achieve the intended therapeutic purpose, such as, to alleviate a sign or symptom of a disease or disorder in a subject.

As used herein a “precursor” and/or “derivative” of psilocybin includes, but is not limited to, prodrugs of psilocybin, prodrugs of an active metabolite of psilocybin, and an active metabolite of psilocybin.

As used herein, a subject that is “psilocybin-naïve” has not previously been exposed to psilocybin.

As used herein, the following Medical Dictionary for Regulatory Activities (MedDRA) terms are considered to be adverse events that are psychedelic in nature: altered mood, altered state of consciousness, autoscopy, delusional perception, disinhibition, dissociation, dissociative identity disorder, dreamy state, emotional disorder, euphoric mood, feeling abnormal, hallucination, hyperacusis, hyperaesthesia, hypoaesthesia, illusion, paranoia, parosmia, photophobia, sensory disturbance, time perception altered, thinking abnormal, synaesthesia, substance-induced psychotic distress, and somatic hallucination.

As used herein, the term “non-responder” refers to a subject or patient population that had no, substantially no, or a negative therapeutic response (e.g., no reduction in depressive symptoms) after administration of psilocybin. A non-responder includes a subject or patient population that exhibits a ≤50% reduction in their baseline Montgomery-Åsberg Depression Rating Scale (MADRS) total score after administration of psilocybin.

As used here, the term “responder” or “sustained responder” refers to a subject or patient population that has a positive therapeutic response (e.g., reduction in depressive symptoms) after administration of psilocybin. A responder includes a subject or patient population that exhibits a ≥50% reduction in their baseline MADRS total score after administration of psilocybin. A sustained responder includes a subject or patient population that exhibits a ≥50% reduction in their baseline MADRS total score after administration of psilocybin and sustains the ≥50% reduction in their baseline MADRS total score over multiple weeks.

### Depressive Disorders

In some embodiments, the methods provided herein are used to treat a subject with a depressive disorder. As used herein, the terms “depressive disorder”, “depression disorder”, or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions.

In some embodiments, the depressive disorder is major depressive disorder, atypical depression, bipolar disorder, catatonic depression, depressive disorder due to a medical condition, postpartum depression, premenstrual dysphoric disorder, or seasonal affective disorder.

As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to-day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all—or almost all—activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide.

Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e. depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g. vitamin B12 deficiency), autoimmune conditions (e.g., lupus and rheumatoid arthritis), viral or other infections (hepatitis, mononucleosis, herpes), back pain, and certain cancers (e.g., pancreatic).

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Medical Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with “treatment-resistant depression”. The term “treatment-resistant depression” refers to a kind of depression (e.g., as described herein) that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, or 5 treatment attempts. In some embodiments, the subject with treatment-resistant depression has been diagnosed with major depressive disorder and has failed to respond to 3 or more treatment attempts. In some embodiments, the subject with treatment-resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of major depressive disorder. In some embodiments, the methods provided herein reduce at least one sign or symptom of major depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of atypical depression. In some embodiments, the methods provided herein reduce at least one sign or symptom of atypical depression by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of bipolar disorder. In some embodiments, the methods provided herein reduce at least one sign or symptom of bipolar disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of bipolar I disorder. In some embodiments, the methods provided herein reduce at least one sign or symptom of bipolar I disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of bipolar II disorder. In some embodiments, the methods provided herein reduce at least one sign or symptom of bipolar II disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of catatonic depression. In some embodiments, the methods provided herein reduce at least one sign or symptom of catatonic depression by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of a depressive disorder due to a medical condition. In some embodiments, the methods provided herein reduce at least one sign or symptom of a depressive disorder due to a medical condition by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of postpartum depression. In some embodiments, the methods provided herein reduce at least one sign or symptom of postpartum depression by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of premenstrual dysphoric disorder. In some embodiments, the methods provided herein reduce at least one sign or symptom of premenstrual dysphoric disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of seasonal affective disorder. In some embodiments, the methods provided herein reduce at least one sign or symptom of seasonal affective disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression is reduced or eliminated in the subject within 1 hour, 2 hours, 3 hours, 6 hours, 12 hours, 24 hours, 48 hours, 3 days, 1 week, 2 weeks, 3 weeks, 4 weeks, 5 weeks, 6 weeks, 7 weeks, 8 weeks, or 3 months following administration of psilocybin or an active metabolite thereof.

In some embodiments, the sign or symptom of depression is reduced or eliminated in the subject for a period of 1 day, 3 days, 7 days, 10 days, about 2 weeks, about 3 weeks, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, 12 months, about 18 months, about 24 months, or about 48 months following administration of psilocybin or an active metabolite thereof. In some embodiments, the sign or symptom of depression is reduced or eliminated in the subject (or a population) for a period (or median period) of about 100 days, about 110 days, about 120 days, about 130 days, about 140 days, about 150 days, about 160 days, about 170 days, about 180 days, about 190 days, about 200 days, about 210 days, about 220 days, about 230 days, about 240 days, or about 250 days. In some embodiments, the sign or symptom of depression is reduced or eliminated in the subject (or population) for a period (or mean period) of about 175 days, about 176 days, about 177 days, about 178 days, about 179 days, about 180 days, about 181 days, about 182 days, about 183 days, about 184 days, about 185 days, about 186 days, about 187 days, about 189 days, about 190 days, about 191 days, about 192 days, about 193 days, about 194 days, about 195 days, about 196 days, about 197 days, about 198 days, about 199 days, about 200 days, about 201 days, about 202 days, about 203 days, about 204 days, or about 205 days, including all values and ranges therein. In some embodiments, there is no recurrence of depression in the subject upon administration of psilocybin or an active metabolite thereof.

In some embodiments, no other treatment is administered to the subject to reduce the sign or symptom of depression after administration of the psilocybin.

In some embodiments, the method of the present disclosure further comprises administering to the subject at least one additional therapeutic to reduce the sign or symptom of depression. In some embodiments, at least one additional therapeutic is a selective serotonin reuptake inhibitor, a serotonin and norepinephrine reuptake inhibitor, a tricyclic antidepressant, a tetracyclic antidepressant, a dopamine reuptake inhibitor, a 5-HT1A receptor antagonist, a 5-HT2 receptor antagonist, a 5-HT3 receptor antagonist, a monoamine oxidase inhibitor, or a noradrenergic antagonist. In some embodiments, at least one additional therapeutic is administered prior to administration of psilocybin, on the same day as the administration of psilocybin, or after administration of psilocybin.

In some embodiments, the subject with the depressive disorder has an additional comorbidity or disorder. In some embodiments, the additional comorbidity or disorder is an anxiety disorder, an obsessive-compulsive disorder, alcoholism, a personality disorder, a cardiovascular disease, a neurological disease, or cancer. In some embodiments, the subject has dementia, Alzheimer's Disease, or Parkinson's Disease. In some embodiments, reducing at least one sign or symptom of depression in the subject using the methods of the present disclosure prevents one or more comorbidities or disorders in the subject.

### Psilocybin

In some embodiments, a method of treatment comprises the administration of a therapeutically effective amount of psilocybin, a prodrug of psilocybin, an active metabolite of psilocybin, or a prodrug of an active metabolite of psilocybin to a subject in need thereof as described herein. In some embodiments, a method of treatment comprises the administration of a therapeutically effective amount of psilocybin as described herein. In some embodiments, a method of treatment comprises the administration of a therapeutically effective amount of psilocin as described herein. Some embodiments comprise psilocybin, a prodrug of psilocybin, an active metabolite of psilocybin, or a prodrug of an active metabolite of psilocybin for use in the treatment of an indication as described herein. Some embodiments comprise psilocybin for use in the treatment of an indication as described herein. Some embodiments comprise psilocin for use in the treatment of an indication as described herein. Some embodiments comprise the use of psilocybin, a prodrug of psilocybin, an active metabolite of psilocybin, or a prodrug of an active metabolite of psilocybin in the manufacture of a medicament for the treatment of an indication as described herein.

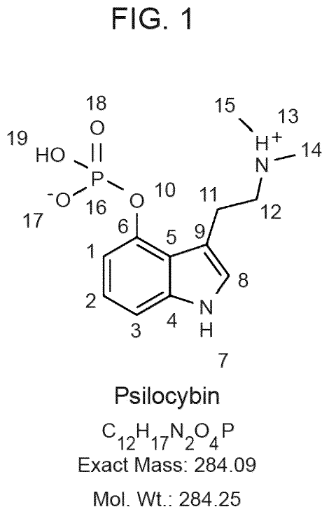

A numbered structural formula of psilocybin is shown in FIG. 1. Novel polymorphs and hydrates of psilocybin, along with the preparation and formulations thereof are disclosed in U.S. Application No. 2019/0119310 A1, which is incorporated by reference herein in its entirety. US2019/0119310 discloses a number of formulations and the challenges of formulating psilocybin due to e.g. its hygroscopicity and poor flow characteristics. US2019/0119310 also discloses the importance of a controlled aqueous crystallisation process.

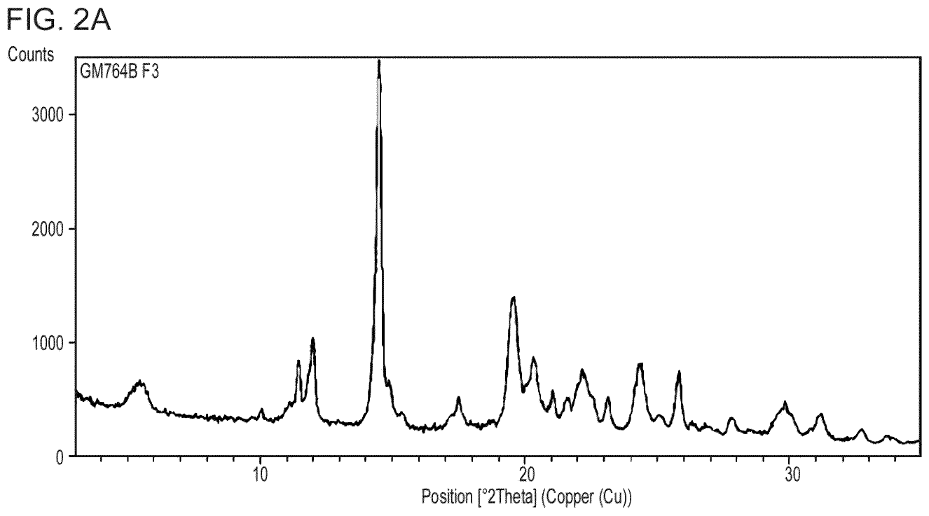

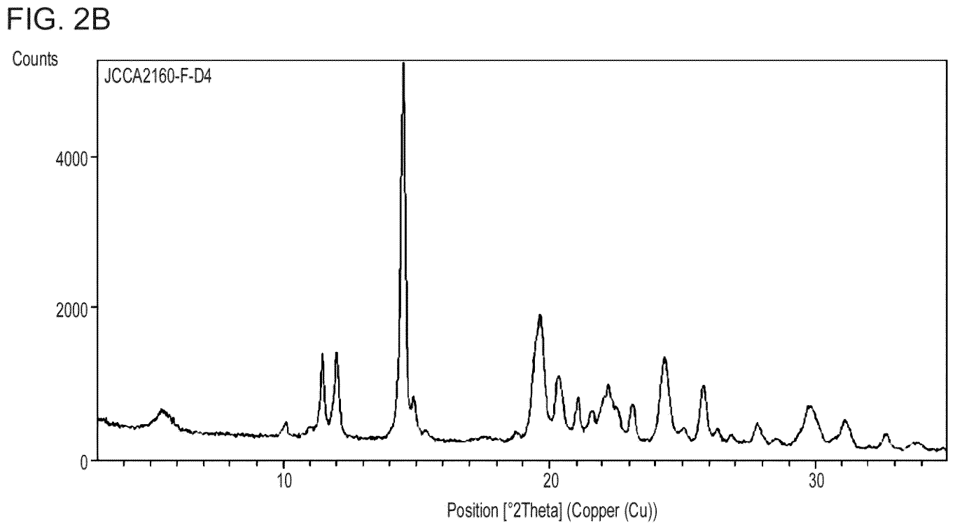

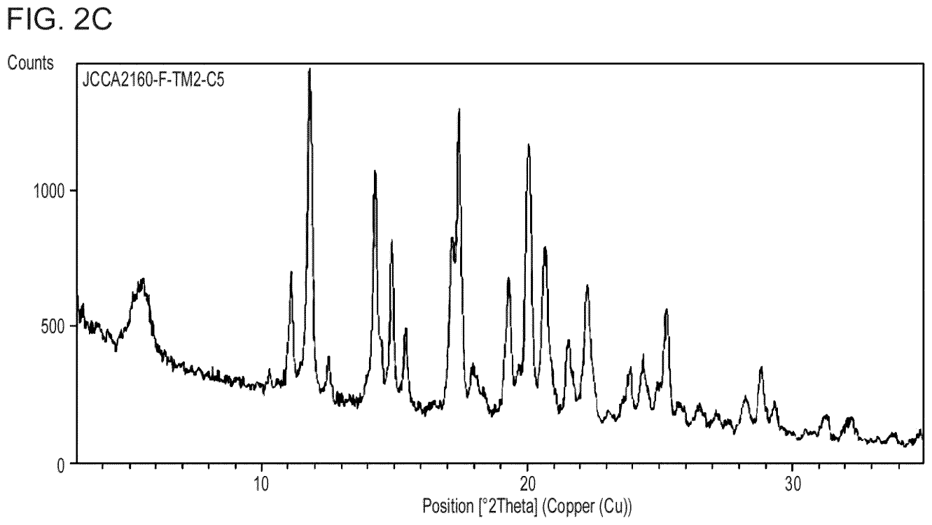

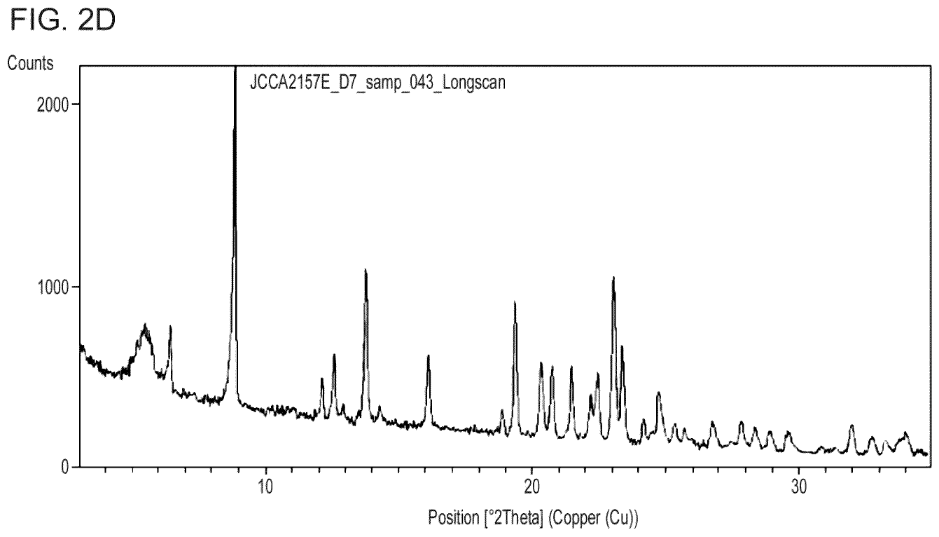

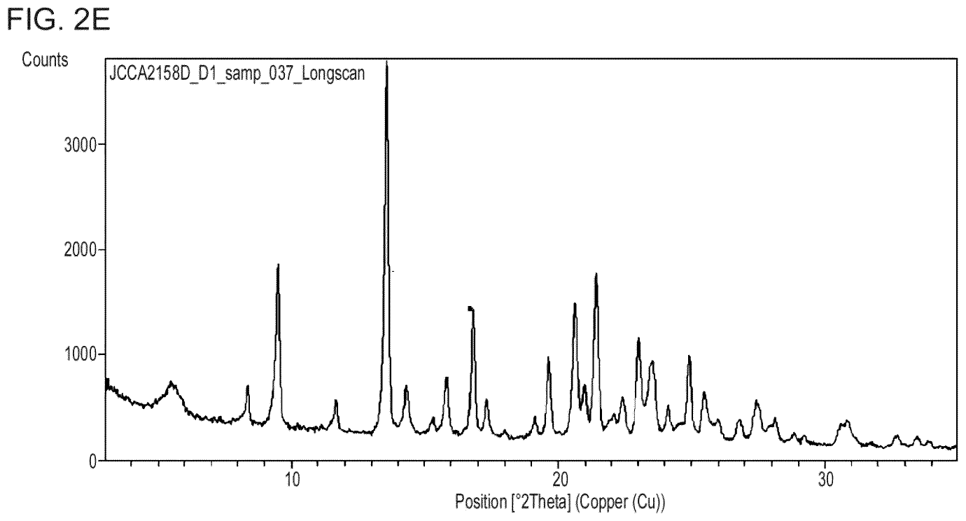

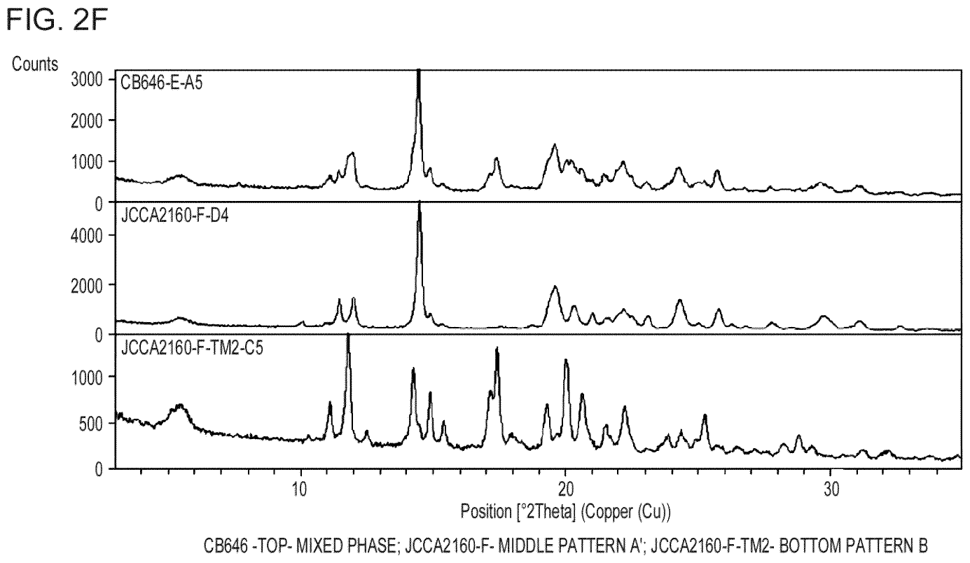

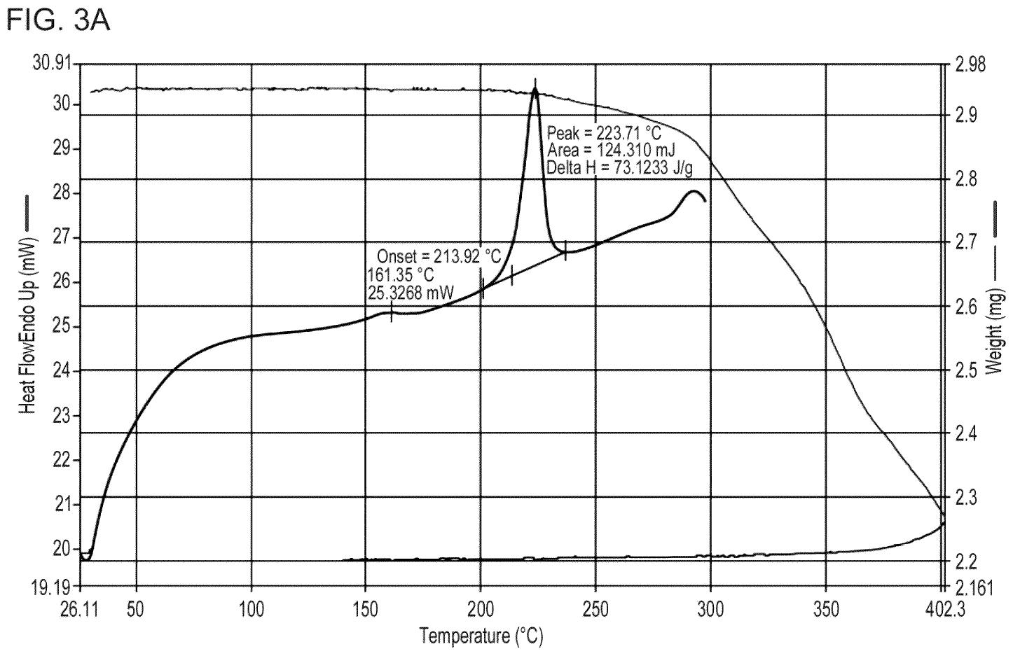

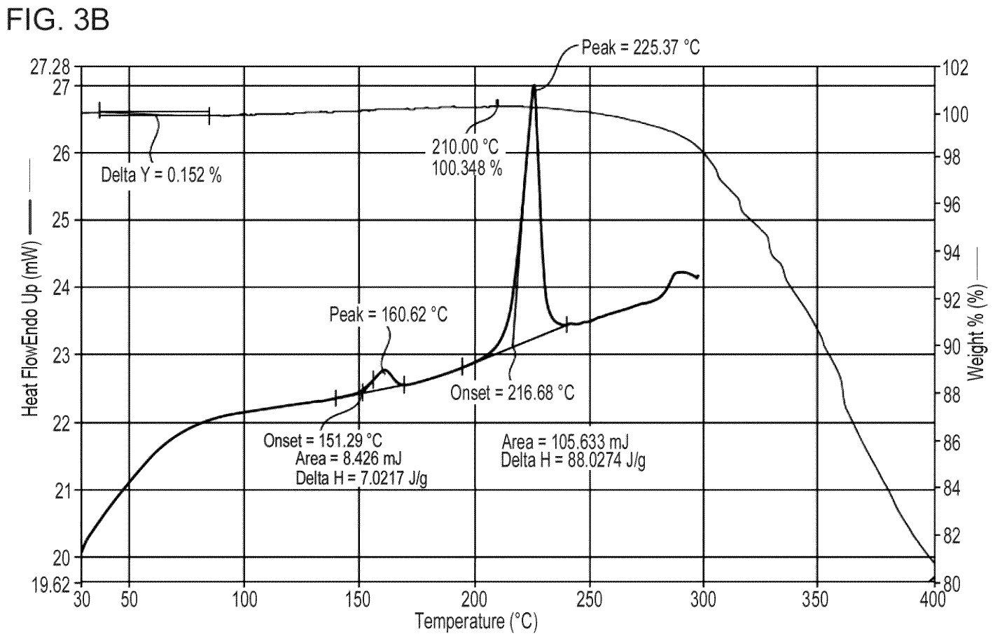

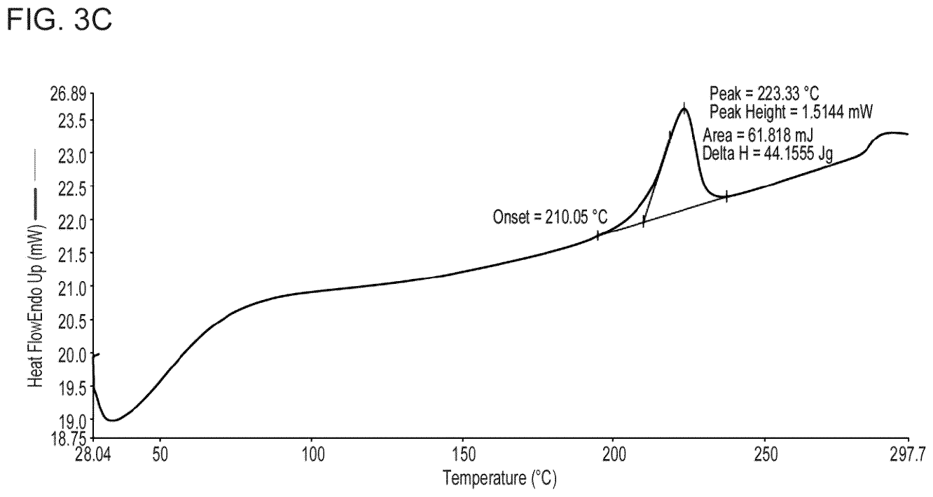

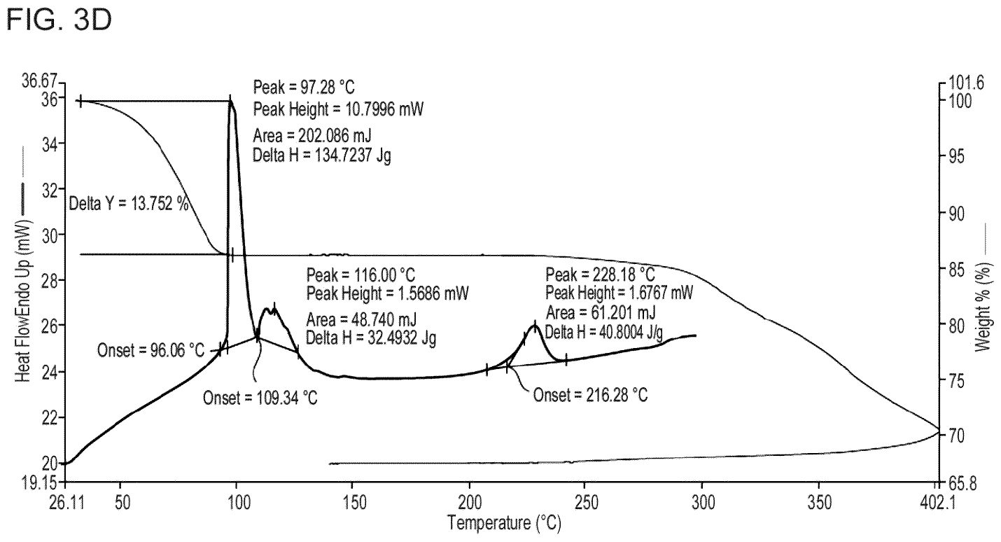

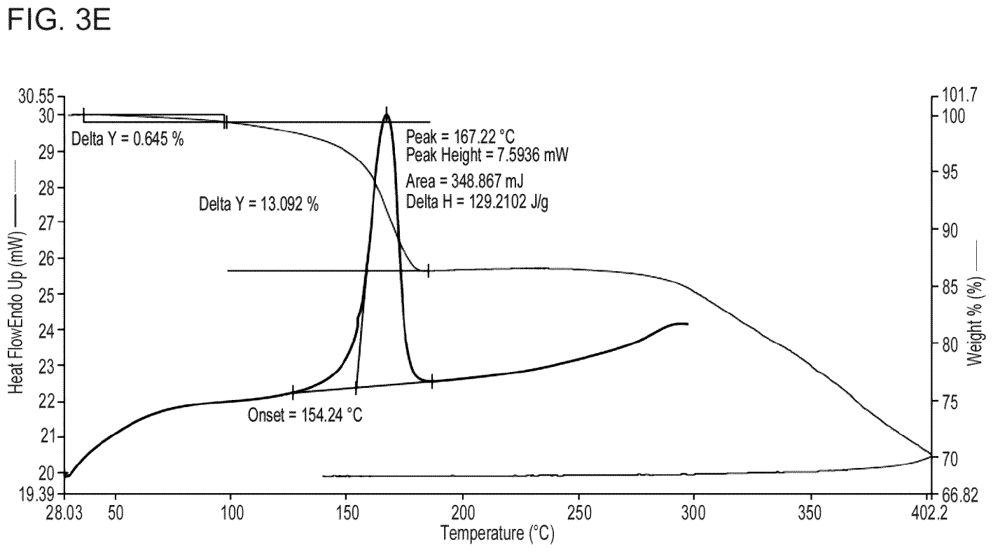

In some embodiments, the psilocybin comprises crystalline psilocybin in the form Polymorph A or Polymorph A′, as described herein, the crystalline psilocybin exhibiting peaks in an X-ray powder diffraction (XRPD) diffractogram at 11.5, 12.0 and 14.5°2θ±0.1°2θ. In some embodiments, the crystalline psilocybin further exhibits at least one peak in the XRPD diffractogram at 19.7, 20.4, 22.2, 24.3 or 25.7°2θ±0.1°2θ. Illustrative XRPD diffractograms are provided as FIGS. 2A and 2B. In some embodiments, the crystalline psilocybin exhibits an endothermic event in a DSC thermogram having a first onset temperature of between 145° C. and 165° C. and a second onset temperature of between 205° C. and 220° C. Illustrative DSC thermograms are provided as FIGS. 3A and 3B.

### Polymorph A

In some embodiments, the present disclosure provides crystalline psilocybin in the form Polymorph A, characterized by one or more of:
peaks in an XRPD diffractogram at 11.5, 12.0, 14.5, and 17.5, °2θ±0.1°2θ;
peaks in an XRPD diffractogram at 11.5, 12.0, 14.5 and 17.5, °2θ±0.1°2θ, further characterized by at least one further peak at 19.7, 20.4, 22.2, 24.3 or 25.7°2θ+0.1°2θ;
an XRPD diffractogram as substantially illustrated in FIG. 2A; or
an endothermic event in a DSC thermogram having an endothermic event in a DSC thermogram having a first onset temperature of between 145° C. and 165° C. and a second onset temperature of between 205° C. and 220° C. substantially as illustrated in FIG. 3A.

In some embodiments, the peak at 17.5°2θ±0.1°2θ has a relative intensity compared to the peak at 14.5°2θ±0.1°2θ of at least 5%, at least 6%, at least 7%, at least 8%, at least 9%, or at least 10%.

In some embodiments, the present disclosure provides crystalline psilocybin in the form Polymorph A, characterized by one or more of:
peaks in an XRPD diffractogram at 11.5, 12.0, 14.5, and 17.5, °2θ±0.2°2θ;
peaks in an XRPD diffractogram at 11.5, 12.0, 14.5 and 17.5, °2θ±0.2°2θ, further characterized by at least one further peak at 19.7, 20.4, 22.2, 24.3 or 25.7°2θ±0.2°2θ;
an XRPD diffractogram as substantially illustrated in FIG. 2A; or
an endothermic event in a DSC thermogram having an endothermic event in a DSC thermogram having a first onset temperature of between 145° C. and 165° C. and a second onset temperature of between 205° C. and 220° C. substantially as illustrated in FIG. 3A.

In some embodiments, the crystalline psilocybin of Polymorph A exhibits an XRPD diffractogram having at least 3, 4, 5, 6, 7, 8, 9, 10, 11, 12, 13, 14, 15, 16, or 17 of the peaks listed in Table A, or equivalent peaks within about +0.1° 20 of the peaks listed in Table A. In some embodiments, the crystalline psilocybin of Polymorph A exhibits an XRPD diffractogram having at least 3, 4, 5, 6, 7, 8, 9, 10, 11, 12, 13, 14, 15, 16, or 17 of the peaks listed in Table A, or equivalent peaks within about ±0.2°2θ of the peaks listed in Table A. In some embodiments, Polymorph A exhibits a peak at 17.5°2θ±0.1°2θ that is substantially absent in Polymorph A′. In some embodiments, Polymorph A exhibits a peak at 17.5°2θ±0.2°2θ that is substantially absent in Polymorph A′.

TABLE A
XRPD peak positions for Polymorph A

| Position [°2 Th.] | Relative Intensity [%] |
|---|---|
| 5.6 | 8.42 |
| 11.5 | 13.05 |
| 12.0 | 26.45 |
| 14.5 | 100.00 |
| 17.5 | 10.71 |
| 19.7 | 37.29 |
| 20.4 | 20.06 |
| 22.2 | 17.83 |
| 23.2 | 6.99 |
| 24.3 | 17.93 |
| 25.7 | 16.40 |
| 26.8 | 3.15 |
| 27.8 | 4.54 |
| 29.7 | 9.53 |
| 31.2 | 6.51 |
| 32.6 | 2.45 |
| 33.7 | 1.75 |

In some embodiments, crystalline psilocybin Polymorph A exhibits XRPD diffractogram peaks at 11.5, 12.0, 14.5, and 17.5°2θ±0.1°2θ. In some embodiments, crystalline psilocybin Polymorph A exhibits at least one additional peak appearing at 19.7, 20.4, 22.2, 24.3 or 25.7°2θ±0.1°2θ. In some embodiments, crystalline psilocybin Polymorph A exhibits at least two additional peaks appearing at 19.7, 20.4, 22.2, 24.3 or 25.7°2θ±0.1°2θ. In some embodiments, crystalline psilocybin Polymorph A exhibits at least three additional peaks appearing at 19.7, 20.4, 22.2, 24.3 or 25.7°2θ±0.1°2θ. In some embodiments, crystalline psilocybin Polymorph A exhibits an XRPD diffractogram substantially the same as the XRPD diffractogram shown in FIG. 2A.

In some embodiments, crystalline psilocybin Polymorph A is characterized by XRPD diffractogram peaks at 14.5 and 17.5°2θ±0.1°2θ with the peak at 17.5°2θ having an intensity which is at least about 5%, at least about 6%, at least about 7%, at least about 8%, at least about 9%, or at least about 10% of the intensity of the peak at 14.5°2θ.

In some embodiments, the crystalline psilocybin Polymorph A exhibits no peak at 10.1—that is, the peak at 10.1 is absent or substantially absent.

In some embodiments, crystalline psilocybin Polymorph A is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C. In some embodiments, crystalline psilocybin Polymorph A exhibits an endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph A further exhibits an endothermic event in the DSC thermogram having an onset temperature of between about 145 and about 165° C., between about 145 and about 160° C., or between about 145 and about 155° C. In some embodiments, crystalline psilocybin Polymorph A exhibits an endothermic event having an onset temperature of between about 205 and about 220° C., between about 210 and about 220° C., between about 210 and about 218° C., or between about 210 and about 216° C.; and an endothermic event having an onset temperature of between about 145 and about 165° C., between about 145 and about 160° C., between about 145 and about 155° C., in a DSC thermogram. In some embodiments, crystalline psilocybin Polymorph A exhibits a DSC thermogram substantially the same as the DSC thermogram in FIG. 3A.

In some embodiments, crystalline psilocybin Polymorph A exhibits a water content of <0.5% w/w, <0.4% w/w, <0.3% w/w, <0.2% w/w, or <0.1% w/w. The water content of a crystalline compound can be determined by known methods, for example Karl Fischer Titration. In some embodiments, crystalline psilocybin Polymorph A exhibits <0.5% w/w loss, <0.4% w/w, <0.3% w/w, <0.2% w/w, or <0.1% w/w in the TGA thermogram between ambient temperature, e.g., about 25° C., and 200° C. In some embodiments, crystalline psilocybin Polymorph A loses less than 2% by weight, less than 1% by weight, or than 0.5% by weight in a loss on drying test, e.g., a loss on drying test performed at 70° C.

In some embodiments, crystalline psilocybin Polymorph A is a highly pure crystalline form of Polymorph A, for example, the in a loss on drying test psilocybin comprises at least 90%, at least 95%, at least 99%, or at least 99.5% by weight crystalline psilocybin of Polymorph A.

In some embodiments, crystalline psilocybin Polymorph A is a white to off-white solid.

In some embodiments, crystalline psilocybin Polymorph A is chemically pure, for example the psilocybin has a chemical purity of greater than 97%, 98%, or 99% by HPLC. In some embodiments, crystalline psilocybin Polymorph A has no single impurity of greater than 1%, greater than 0.5%, greater than 0.4%, greater than 0.3%, or greater than 0.2% e.g., the impurity phosphoric acid as measured by ³¹P NMR, or the impurity psilocin measured by HPLC. In some embodiments, crystalline psilocybin Polymorph A has a chemical purity of greater than 97 area %, greater than 98 area %, or greater than 99 area % by HPLC. In some embodiments, crystalline psilocybin Polymorph A has no single impurity greater than 1 area %, greater than 0.5 area %, greater than 0.4%, greater than 0.3%, or greater than 0.2% as measured by HPLC. In some embodiments, crystalline psilocybin Polymorph A does not contain psilocin at a level greater than 1 area %, greater than 0.5 area %, greater than 0.4%, greater than 0.3%, or greater than 0.2% as measured by HPLC. In some embodiments, crystalline psilocybin Polymorph A does not contain phosphoric acid at a level greater than 1 weight %, greater than 0.5 weight %, greater than 0.4 weight %, 0.3 weight %, or greater than 0.2 weight %, as measured by ³¹P NMR. In some embodiments, crystalline psilocybin Polymorph A has a chemical assay of at least 95 weight %, at least 96 weight %, or at least 98 weight %.

### Methods of Manufacturing Crystalline Psilocybin Polymorph A.

In another embodiment, the disclosure provides a method for large scale manufacture of psilocybin characterized in that the method comprises subjecting psilocybin to a water crystallization step, with controlled drying, to produce crystalline psilocybin Polymorph A.

In another embodiment, the disclosure provides a method for large scale manufacture of psilocybin characterized in that the method comprises subjecting psilocybin to a water crystallization step, with controlled drying, to produced crystalline psilocybin Polymorph A with an XRPD diffractogram as illustrated in FIG. 2A and a DSC and TGA thermograph as illustrated in FIG. 3a. In another embodiment, the disclosure provides a method for large-scale manufacture of psilocybin characterized in that the method comprises subjecting psilocybin to a water crystallization step, with controlled drying, to produce a high purity crystalline psilocybin-Polymorph A with an XRPD diffractogram as illustrated in FIG. 2a and a DSC thermograph as illustrated in FIG. 3a.

In another embodiment of the disclosure, psilocybin is recrystallized in about 10-20 volumes of water, heated with agitation to a temperature of at least 70° C., polish filtered with a suitable cut off (typically, below 5 μm), seeded at a temperature of about 70° C., and cooled in a controlled manner to about 5° C. over a period of more than 2 hours.

In some embodiments, psilocybin recrystallization comprises controlled cooling which drops the temperature by about 5° C.-15° C. an hour, more preferably about 10° C. an hour. In certain embodiments, the polish filter step is done through an appropriately sized filter such as, but not limited to, a 1.2 μm in line filter.

In some embodiments, agitation is by stirring at about 400-500 rpm, typically about 450 rpm.

In some embodiments, the psilocybin is dissolved in water heated to no more than 90° C. In some embodiments the psilocybin is dissolved in water heated to no more than 85° C. Without being bound by any particular mechanism, this dissolution step is intended to solubilize psilocybin whilst also minimizing the formation of hydrolysis products.

In some embodiments, the psilocybin solution is stirred to speed the solubilization and reduce the time that the solution is at a high temperature, namely one at or around 80° C., or higher.

In some embodiments, the seed is psilocybin Hydrate A. In one embodiment, 0.1% weight or less of seed is added to the process.

In some embodiments, the psilocybin the crystalline psilocybin is isolated by vacuum filtration.

In some embodiments, the isolated crystals are dried in vacuo at a temperature of at least 30° C., such as between 3° and 50° C., or such as between 4° and 50° C. In some embodiment, the isolated crystals are dried in vacuo for at least 10 hours, such as between 12 and 18 hours, or such as about 30 hours. In some embodiments, the isolated crystals are dried in vacuo at a temperature of at least 30° C., such as between 3° and 50° C., or such as between 4° and 50° C., for at least 10 hours, such as between 12 and 18 hours, or such as about 30 hours. In some embodiments, the isolated crystals are dried until the isolated crystals lose less than 2% weight in a loss on drying test, such as less than 0.5% weight.

In some embodiments, the isolated crystals are washed, several times, in water and dried in vacuo at about 50° C. for at least 12 hours.

In some embodiments, the crystals obtained are typically relatively large (range 50 to 200 microns) and uniform when viewed under the microscope ×10.

In contrast, crystals obtained without controlled cooling which are much smaller in size (typically 5 to 50 microns) when viewed under the microscope ×10.

In some embodiments, there is provided Psilocybin obtained by the method of crystallization described herein.

In some embodiments, there is provided a pharmaceutical formulation comprising psilocybin polymorph A obtained by the method of crystallization described herein.

In some embodiments, psilocybin manufactured prior to crystallization may be produced using one of the following methods: synthetic or biological, e.g. by fermentation or obtained by extraction from mushrooms. In some embodiments, psilocybin manufactured prior to crystallization is manufactured according to all or some of the methods described in U.S Application No. 2019/0119310 A1, which is incorporated by reference herein in its entirety.

### Polymorph A′

The present disclosure provides crystalline psilocybin in the form of Polymorph A′, characterized by one or more of:
(i) peaks in an XRPD diffractogram at 11.5, 12.0 and 14.5°2θ+0.1°2θ, but absent or substantially absent of a peak at 17.5°2θ±0.1°2θ;
(ii) peaks in an XRPD diffractogram at 11.5, 12.0 and 14.5°2θ±0.1°2θ, but absent or substantially absent of a peak at 17.5°2θ+0.1°2θ, further characterized by at least one further peak at 19.7, 20.4, 22.2, 24.3 or 25.7°2θ±0.1°2θ;
(iii) an XRPD diffractogram as substantially illustrated in FIG. 2B; or
(iv) an endothermic event in a DSC thermogram having a first onset temperature of between 145° C. and 165° C. and a second onset temperature of between 205° C. and 220° C. substantially as illustrated in FIG. 3B.

In some embodiments, the crystalline psilocybin comprises crystalline psilocybin Polymorph A′. Crystalline psilocybin Polymorph A′ exhibits peaks in an XRPD diffractogram at 11.5, 12.0 and 14.5°2θ±0.1°2θ, but absent or substantially absent of a peak at 17.5°2θ+0.1°2θ. In some embodiments, crystalline psilocybin Polymorph A′ further exhibits 1, 2, 3, 4, or 5 peaks selected from 19.7, 20.4, 22.2, 24.3 or 25.7°2θ±0.1°2θ. An illustrative XRPD diffractogram for Polymorph A′ is provided as FIG. 2B. An illustrative DSC thermogram having an onset temperature of between 205 and 220° C. for Polymorph A′ is provided as FIG. 3B.

In some embodiments, psilocybin Polymorph A′ exhibits an XRPD diffractogram as summarized in Table B. In some embodiments, crystalline psilocybin Polymorph A′ exhibits at least 3, 4, 5, 6, 7, 8, 9, 10, 11, 12, 13, 14, 15, 16, 17, 18, 19, 20, 21, 22, 23, 24, or 25 peaks listed of Table B or equivalent peaks within about +0.1°2θ, and absent or substantially absent peak at 17.5°2θ±0.1°2θ.

TABLE B
XRPD peak positions for Polymorph A′

| Position [°2 Th.] | Relative Intensity [%] |
|---|---|
| 5.5 | 4.89 |
| 10.1 | 4.09 |
| 11.5 | 22.05 |
| 12.0 | 22.77 |
| 14.5 | 100.00 |
| 14.9 | 11.29 |
| 17.5 | 1.08 |
| 18.7 | 2.44 |
| 19.4 | 23.02 |
| 19.6 | 33.70 |
| 20.3 | 17.01 |
| 21.1 | 12.08 |
| 21.6 | 8.51 |
| 22.2 | 15.54 |
| 22.6 | 8.78 |
| 23.1 | 10.11 |
| 24.3 | 21.83 |
| 25.1 | 4.36 |
| 25.8 | 15.40 |
| 26.3 | 4.28 |
| 26.8 | 2.86 |
| 27.8 | 5.96 |
| 28.6 | 1.91 |
| 29.7 | 10.56 |
| 31.1 | 7.35 |
| 32.6 | 3.72 |
| 33.8 | 1.54 |

In some embodiments, crystalline psilocybin Polymorph A′ exhibits XRPD diffractogram peaks at 11.5, 12.0, and 14.5°2θ±0.1°2θ but substantially absent of a peak at 17.5°2θ±0.1°2θ. In some embodiments, crystalline psilocybin Polymorph A′ further exhibits at least one additional peak appearing at 19.7, 20.4, 22.2, 24.3, or 25.7° 2θ±0.1°2θ. In some embodiments, crystalline psilocybin Polymorph A′ exhibits at least two additional peaks appearing at 19.7, 20.4, 22.2, 24.3, or 25.7°2θ±0.1°2θ. In some embodiments, crystalline psilocybin Polymorph A′ exhibits and is distinguished from Polymorph A by the presence of a peak appearing at 10.1°2θ+0.1°2θ. In yet a further embodiment, crystalline psilocybin Polymorph A′ exhibits an XRPD diffractogram substantially the same as the XRPD diffractogram shown in FIG. 2B.

In some embodiments, crystalline psilocybin Polymorph A′ exhibits XRPD diffractogram peaks at 14.5 and 17.5°2θ±0.1°2θ, wherein the intensity of the peak at 17.5°2θ is less than 5%, less than 4%, less than 3%, less than 2%, or less than 1% of the intensity of the peak at 14.5°2θ.

In some embodiments, crystalline psilocybin Polymorph A′ exhibits XRPD diffractogram peaks at 10.1 and 14.5°2θ±0.1°2θ, wherein the intensity of the peak at 10.1°2θ is at least 1%, at least 2%, at least 3%, or at least 4% of the intensity of the peak at 14.5°2θ.

In some embodiments, crystalline psilocybin Polymorph A′ is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C. In some embodiments, crystalline psilocybin Polymorph A′ is characterized by an endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph A′ exhibits an endothermic event in the DSC thermogram having an onset temperature of between about 145 and about 165° C., between about 145 and about 160° C., or between about 145 and about 155° C. In some embodiments, crystalline psilocybin Polymorph A′ exhibits an endothermic event having an onset temperature of between about 205 and about 220° C., between about 210 and about 220° C., between about 210 and about 218° C., or between about 210 and about 216° C., and an endothermic event having an onset temperature of between about 145 and about 165° C., between about 145 and about 160° C., or between about 145 and about 155° C., in a DSC thermogram. In some embodiments, crystalline psilocybin Polymorph A′ exhibits a DSC thermogram substantially the same as the DSC thermogram in FIG. 3B.

In some embodiments, crystalline psilocybin Polymorph A′ exhibits a water content of <0.5% w/w, <0.4% w/w, <0.3% w/w, <0.2% w/w, or <0.1% w/w. Methods to determine the water content of a crystalline compound are known, for example Karl Fischer Titration. In some embodiments, crystalline psilocybin Polymorph A′ exhibits <0.5% w/w loss, <0.4% w/w, <0.3% w/w, <0.2% w/w, <0.1% w/w in the TGA thermogram between ambient temperature, e.g., 25° C., and 200° C. In some embodiments, crystalline psilocybin Polymorph A′ loses less than 2% by weight, less than 1% by weight, or less than 0.5% by weight in a loss on drying test. In some embodiments, the loss on drying test is performed at 70° C.

In some embodiments, crystalline psilocybin Polymorph A′ is a highly pure crystalline form of Polymorph A′. In some embodiments, the crystalline psilocybin comprises at least 90%, 95%, 99%, or 99.5% by weight of Polymorph A′.

In some embodiments, crystalline psilocybin Polymorph A's is a white to off white solid.

In some embodiments, crystalline psilocybin Polymorph A′ is chemically pure, for example the psilocybin has a chemical purity of greater than 97%, greater than 98%, or than 99% by HPLC. In some embodiments, crystalline psilocybin Polymorph A′ has no single impurity of greater than 1% or greater than 0.5%, e.g., the impurity phosphoric acid as measured by 31P NMR or the impurity psilocin as measured by HPLC. In some embodiments, crystalline psilocybin Polymorph A′ has a chemical purity of greater than 97 area %, greater than 98 area %, or greater than 99 area % by HPLC. In some embodiments, crystalline psilocybin Polymorph A′ has no single impurity greater than 1 area % or greater than 0.5 area %, e.g., as measured by HPLC. In some embodiments, crystalline psilocybin Polymorph A′ does not contain psilocin at a level greater than 1 area % or greater than 0.5 area % as measured by HPLC. In some embodiments, crystalline psilocybin Polymorph A′ does not contain phosphoric acid at a level greater than 1 weight % or greater than 0.5 weight % as measured by 31P NMR. In some embodiments, crystalline psilocybin Polymorph A′ has a chemical assay of at least 95 weight %, at least 96 weight %, or at least 98 weight %.

In some embodiments, crystalline psilocybin Polymorph A′ is chemically pure, for example the psilocybin has a chemical purity of greater than 97%, 98%, or 99% by HPLC. In some embodiments, crystalline psilocybin Polymorph A′ has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Polymorph A′ has a chemical purity of greater than 97 area %, greater than 98 area %, or greater than 99 area % by HPLC. In some embodiments, crystalline psilocybin Polymorph A′ has no single impurity greater than 1 area %, greater than 0.5 area %, greater than 0.4%, greater than 0.3%, or greater than 0.2% as measured by HPLC. In some embodiments, crystalline psilocybin Polymorph A′ does not contain psilocin at a level greater than 1 area %, greater than 0.5 area %, greater than 0.4%, greater than 0.3%, or greater than 0.2% as measured by HPLC. In some embodiments, crystalline psilocybin Polymorph A′ does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph A′ has a chemical assay of at least 95 weight %, at least 96 weight %, or at least 98 weight %.

Illustrative XRPD diffractograms for high purity crystalline psilocybin, Polymorph A or Polymorph A′ are provided in FIGS. 2A and 2B. Illustrative DSC thermographs for high purity crystalline psilocybin, Polymorph A or Polymorph A′ are provided in FIGS. 2A and 2B.

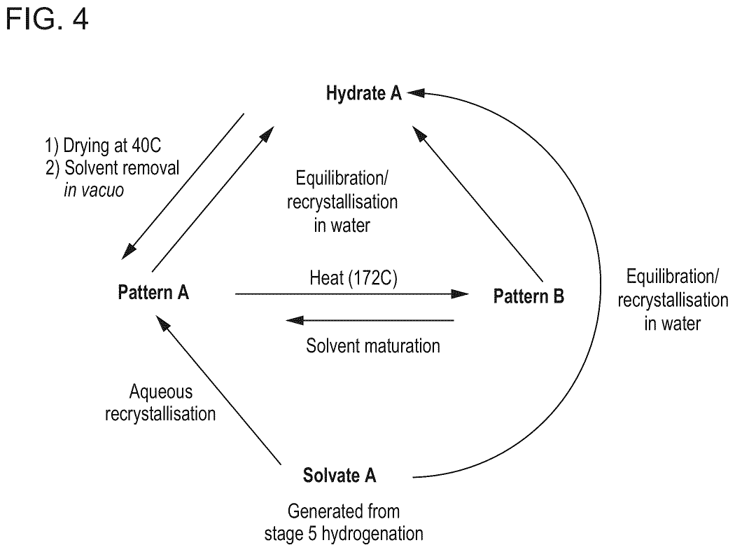

Polymorph A (including its isostructural variant Polymorph A′) (FIGS. 2A and 2B) differs from Polymorph B (FIG. 2C), the Hydrate A (FIG. 2D) and the ethanol solvate (FIG. 2E: Solvate A), and the relationship between some of the different forms is illustrated in FIG. 4.

In some embodiments, the crystalline psilocybin Polymorph A or Polymorph A′ is a white to off white solid, and/or has a chemical purity of greater than 97%, 98%, or 99% by HPLC. In some embodiments, crystalline psilocybin Polymorph A or Polymorph A′ has no single impurity of greater than 1%, greater than 0.5%, greater than 0.4%, greater than 0.3%, or greater than 0.2% e.g., the impurity phosphoric acid as measured by ³¹P NMR, or the impurity psilocin measured by HPLC. In some embodiments, crystalline psilocybin Polymorph A or Polymorph A′ has a chemical purity of greater than 97 area %, greater than 98 area %, or greater than 99 area % by HPLC. In some embodiments, crystalline psilocybin Polymorph A or Polymorph A′ has no single impurity greater than 1 area %, greater than 0.5 area %, greater than 0.4%, greater than 0.3%, or greater than 0.2% as measured by HPLC. In some embodiments, crystalline psilocybin Polymorph A or Polymorph A′ does not contain psilocin at a level greater than 1 area %, greater than 0.5 area %, greater than 0.4%, greater than 0.3%, or greater than 0.2% as measured by HPLC. In some embodiments, crystalline psilocybin Polymorph A or Polymorph A′ does not contain phosphoric acid at a level greater than 1 weight %, greater than 0.5 weight %, greater than 0.4 weight %, 0.3 weight %, or greater than 0.2 weight %, as measured by ³¹P NMR. In some embodiments, crystalline psilocybin Polymorph A or Polymorph A′ has a chemical assay of at least 95 weight %, at least 96 weight %, or at least 98 weight %.

The heating of Polymorph A or A′ results in an endothermic event having an onset temperature of circa 150° C. corresponding to solid-solid transition of Polymorph A or Polymorph A′ to Polymorph B. Continued heating of the resulting solid, i.e., Polymorph B, results in a second endothermic event corresponding to a melting point having an onset temperature of between 205 and 220° C. (see FIGS. 3A and 3B).

### Hydrate A

In some embodiments, the disclosure provides a crystalline form of psilocybin, Hydrate A. In some embodiments, crystalline psilocybin Hydrate A exhibits peaks in an XRPD diffractogram at 8.9, 12.6 and 13.8°2θ±0.1°2θ. In some embodiments, crystalline psilocybin Hydrate A further exhibits at least 1, 2, 3, 4, or 5 further peaks at 6.5, 12.2, 19.4, 20.4 or 20.8°2θ±0.1°2θ. An illustrative XRPD diffractogram is provided as FIG. 2D. In some embodiments, crystalline psilocybin Hydrate A further exhibits an endothermic event in a DSC thermogram having a first onset temperature of between 90° C. and 100° C., a second onset temperature of between 100° C. and 120° C. and a third onset temperature of between 210° C. and 220° C. An illustrative DSC thermogram is provided as FIG. 2D.

In some embodiments, psilocybin Hydrate A exhibits an XRPD diffractogram comprising at least 3, 4, 5, 6, 7, 8, 9, or 10 peaks listed in Table C or equivalent peaks within about +0.1°2θ.

TABLE C
XRPD peak positions for Hydrate A

| Position [°2 Th.] | Relative Intensity [%] |
|---|---|
| 5.6 | 14.40 |
| 6.5 | 18.84 |
| 8.9 | 100.00 |
| 12.2 | 11.51 |
| 12.6 | 18.65 |
| 13.8 | 44.22 |
| 16.2 | 21.22 |
| 18.9 | 6.62 |
| 19.4 | 38.68 |
| 20.4 | 21.32 |
| 20.8 | 19.73 |
| 21.5 | 20.75 |
| 22.3 | 12.80 |
| 22.5 | 19.38 |
| 23.1 | 47.53 |
| 23.5 | 25.79 |
| 24.3 | 5.62 |
| 24.8 | 14.62 |
| 25.4 | 5.27 |
| 26.9 | 6.53 |
| 27.9 | 7.82 |
| 28.4 | 5.78 |
| 29.0 | 5.09 |
| 29.7 | 4.83 |
| 32.1 | 8.27 |
| 32.8 | 4.81 |
| 33.4 | 3.74 |
| 34.2 | 5.96 |

In some embodiments, crystalline psilocybin Hydrate A exhibits XRPD diffractogram peaks at 8.9, 12.6 and 13.8°2θ±0.1°2θ. In some embodiments, crystalline psilocybin Hydrate A exhibits at least one peak appearing at 6.5, 12.2, 19.4, 20.4 or 20.8°2θ±0.1°2θ. In some embodiments, crystalline psilocybin Hydrate A exhibits at least two peaks appearing at 6.5, 12.2, 19.4, 20.4 or 20.8°2θ±0.1°2θ. In some embodiments, crystalline psilocybin Hydrate A exhibits an XRPD diffractogram substantially the same as the XRPD diffractogram shown in FIG. 2D.

In certain embodiments, crystalline psilocybin Hydrate A is characterized by an endothermic event in a DSC thermogram having a first onset temperature of between 85° C. and 105° C., such as between 90° C. and 100° C. and most preferably at about 96° C., a second onset temperature of between 100° C. and 120° C. such as between 105° C. and 115° C., and most preferably at about 109° C. and a third onset temperature of between 205 and 220° C., such as between 21° and 220° C., such as between 21° and 218° C., or such as between 21° and 216° C., or about 216° C. In some embodiments, crystalline psilocybin Hydrate A exhibits an endothermic event in a DSC thermogram having an onset temperature of between about 205 and about 220° C., between about 210 and about 220° C., between about 210 and about 218° C., or between about 210 and about 216° C. In some embodiments, crystalline psilocybin Hydrate A exhibits an endothermic event in the DSC thermogram having an onset temperature of between about 85 and about 105° C., or between about 90 and about 100° C. In some embodiments, crystalline psilocybin Hydrate A exhibits an endothermic event having an onset temperature of between about 205 and about 220° C., between about 210 and about 220° C., between about 210 and about 218° C., or between about 210 and about 216° C., and an endothermic event having an onset temperature of between about 85 and about 105° C. or between about 90 and about 100° C., in a DSC thermogram. In some embodiments, crystalline psilocybin Hydrate A exhibits a DSC thermogram substantially the same as the DSC thermogram in FIG. 3D.

In some embodiments, crystalline psilocybin Hydrate A exhibits a water content of between about 10 and about 18%, between about 12 and about 16%, or about 13%. Methods to determine the water content of a crystalline compound are known, for example Karl Fischer Titration. In some embodiments, crystalline psilocybin Hydrate A exhibits a weight loss in the TGA thermogram of between about 10 and about 18%, between about 12 and about 16%, or about 13%, between ambient temperature, about 25° C., and 120° C.

In some embodiments, crystalline psilocybin Hydrate A is chemically pure, for example the psilocybin has a chemical purity of greater than 97%, 98%, or 99% by HPLC. In some embodiments, crystalline psilocybin Hydrate A has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Hydrate A has a chemical purity of greater than 97 area %, greater than 98 area %, or greater than 99 area % by HPLC. In some embodiments, crystalline psilocybin Hydrate A has no single impurity greater than 1 area %, greater than 0.5 area %, greater than 0.4%, greater than 0.3%, or greater than 0.2% as measured by HPLC. In some embodiments, crystalline psilocybin Hydrate A does not contain psilocin at a level greater than 1 area %, greater than 0.5 area %, greater than 0.4%, greater than 0.3%, or greater than 0.2% as measured by HPLC. In some embodiments, crystalline psilocybin Hydrate A does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Hydrate A has a chemical assay of at least 95 weight %, at least 96 weight %, or at least 98 weight %.

In some embodiments, crystalline psilocybin Hydrate A is a highly pure crystalline form of Hydrate A. In some embodiments, the crystalline psilocybin comprises at least 90%, at least 95%, at least 99%, or at least 99.5% by weight of Hydrate A.

### Polymorph B

In some embodiments, the disclosure provides a crystalline form of psilocybin, Polymorph B. In some embodiments, crystalline psilocybin Polymorph B exhibits peaks in an XRPD diffractogram at 11.1, 11.8 and 14.3°2θ±0.1°2θ. In some embodiments, crystalline psilocybin Polymorph B exhibits at least 1, 2, 3, 4 or 5 peaks in an XRPD diffractogram at 14.9, 15.4, 19.3, 20.0 or 20.6°2θ±0.1°2θ. An illustrative XRPD diffractogram of crystalline psilocybin Polymorph B is provided as FIG. 2C. In some embodiments, crystalline psilocybin Polymorph B exhibits a single endothermic event in a DSC thermogram having an onset temperature of between about 205 and about 220° C. An illustrative DSC thermogram of crystalline psilocybin Polymorph B is provided as FIG. 3C.

In some embodiments, psilocybin Polymorph B exhibits an XRPD diffractogram comprising at least 3, 4, 5, 6, 7, 8, 9, or 10 peaks listed in Table D or equivalent peaks within about ±0.1°2θ.

TABLE D
XRPD peak positions for Polymorph B

| Position [°2 Th.] | Relative Intensity [%] |
|---|---|
| 5.5 | 21.33 |
| 11.1 | 36.91 |
| 11.8 | 100.00 |
| 12.5 | 12.73 |
| 14.3 | 70.23 |
| 14.9 | 50.01 |
| 15.4 | 23.67 |
| 17.1 | 51.58 |
| 17.4 | 91.25 |
| 18.0 | 12.61 |
| 19.3 | 39.33 |
| 20.0 | 76.61 |
| 20.6 | 50.26 |
| 21.5 | 20.77 |
| 22.3 | 40.19 |
| 23.9 | 13.32 |
| 24.3 | 16.03 |
| 25.3 | 32.94 |
| 28.3 | 7.60 |
| 28.9 | 17.89 |
| 29.3 | 8.96 |
| 31.3 | 6.57 |
| 32.2 | 6.90 |
| 33.8 | 2.37 |

In some embodiments, crystalline psilocybin Polymorph B exhibits XRPD diffractogram peaks at 11.1, 11.8 and 14.3°2θ±0.1°2θ. In some embodiments, crystalline psilocybin Polymorph B exhibits at least one peak at 14.9, 15.4, 19.3, 20.0 or 20.6°2θ+0.1°2θ. In some embodiments, crystalline psilocybin Polymorph B exhibits at least two peaks appearing at 14.9, 15.4, 19.3, 20.0 or 20.6°2θ±0.1°2θ. In some embodiments, crystalline psilocybin Polymorph B exhibits an XRPD diffractogram substantially the same as the XRPD diffractogram shown in FIG. 2C.

In some embodiments, crystalline psilocybin Polymorph B is characterized by a single endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph B exhibits a DSC thermogram substantially the same as the DSC thermogram in FIG. 3C.

In some embodiments, crystalline psilocybin Polymorph B exhibits a water content of <0.5% w/w, <0.4% w/w, <0.3% w/w, <0.2% w/w, or <0.1% w/w. Methods to determine the water content of a crystalline compound are known, for example Karl Fischer Titration. In some embodiments, crystalline psilocybin Polymorph B exhibits <0.5% w/w, <0.4% w/w, <0.3% w/w, <0.2% w/w, or <0.1% w/w loss in the TGA thermogram between ambient temperature, about 25° C., and 200° C. In some embodiments, crystalline psilocybin Polymorph B exhibits a loss of less than 2% by weight, less than 1% by weight, or less than 0.5% by weight in a loss on drying test. In some embodiments, the loss on drying test is performed at 70° C.

In some embodiments, crystalline psilocybin Polymorph B is a highly pure crystalline form of Polymorph B, for example, psilocybin comprises at least 90%, at least 95%, at least 99%, or at least 99.5% by weight of Polymorph B.

In some embodiments, crystalline psilocybin Polymorph B is chemically pure, for example the psilocybin has a chemical purity of greater than 97%, 98%, or 99% by HPLC. In some embodiments, crystalline psilocybin Polymorph B has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Polymorph B has a chemical purity of greater than 97 area %, greater than 98 area %, or greater than 99 area % by HPLC. In some embodiments, crystalline psilocybin Polymorph B has no single impurity greater than 1 area %, greater than 0.5 area %, greater than 0.4%, greater than 0.3%, or greater than 0.2% as measured by HPLC. In some embodiments, crystalline psilocybin Polymorph B does not contain psilocin at a level greater than 1 area %, greater than 0.5 area %, greater than 0.4%, greater than 0.3%, or greater than 0.2% as measured by HPLC. In some embodiments, crystalline psilocybin Polymorph B does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph B has a chemical assay of at least 95 weight %, at least 96 weight %, or at least 98 weight %.

In some embodiments, the psilocybin of the disclosure in the form Polymorph A or A′ has the general properties illustrated in Table D.

TABLE D

| | |
|---|---|
| Appearance: | White to off-white solid |
| Major endothermic event in | 210-215° C. |
| DSC (onset temperature) | |
| (corresponding to a melt): | |
| Hygroscopicity: | Psilocybin forms Hydrate A at high |
| | humidity and when added to water |
| | but the water of hydration is lost |
| | rapidly on drying. The anhydrous |
| | form is therefore being developed. |
| Crystalline form: | Anhydrous Polymorph A and/or A′ |
| pKa (calculated): | 1.74, 6.71, 9.75 |
| Solubility | approx. 15 mg/ml in Water |

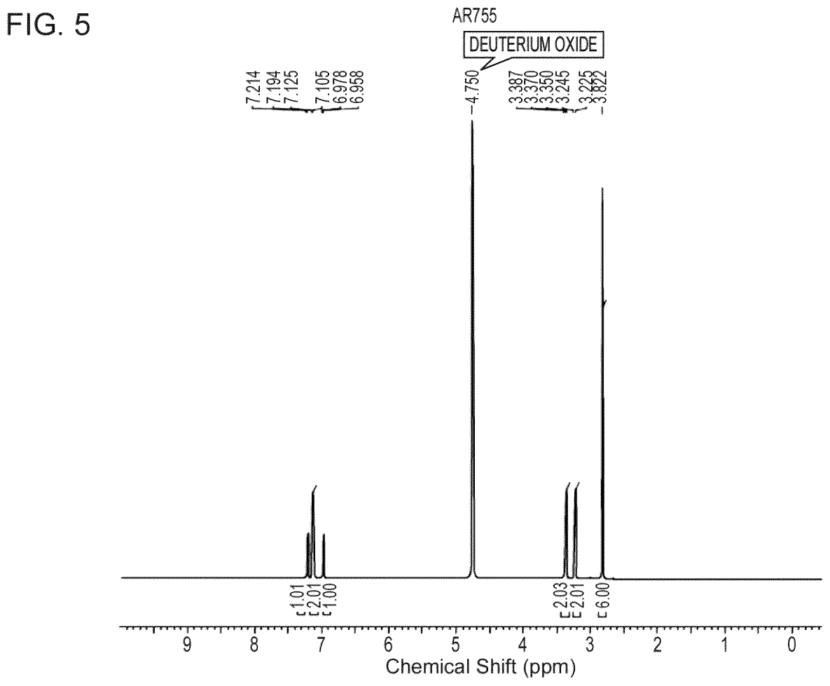

In some embodiments, the psilocybin conforms to the spectra as set out in Table E and illustrated in the spectra of FIGS. 5-8.

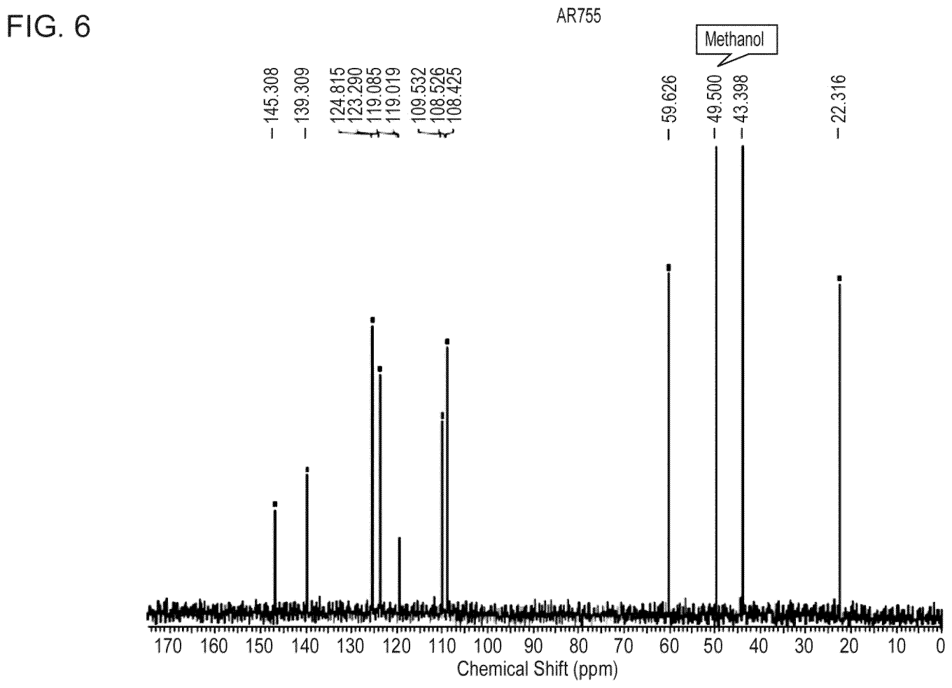

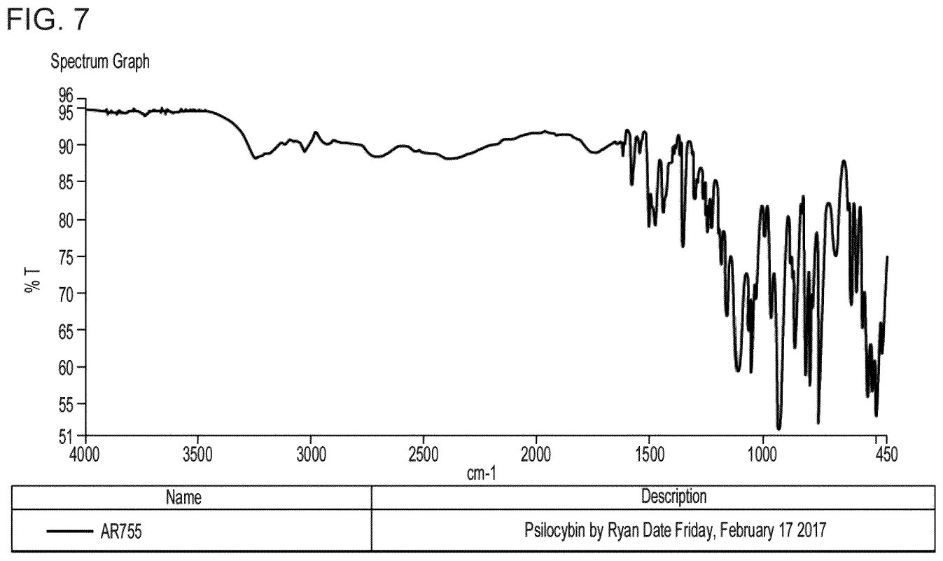

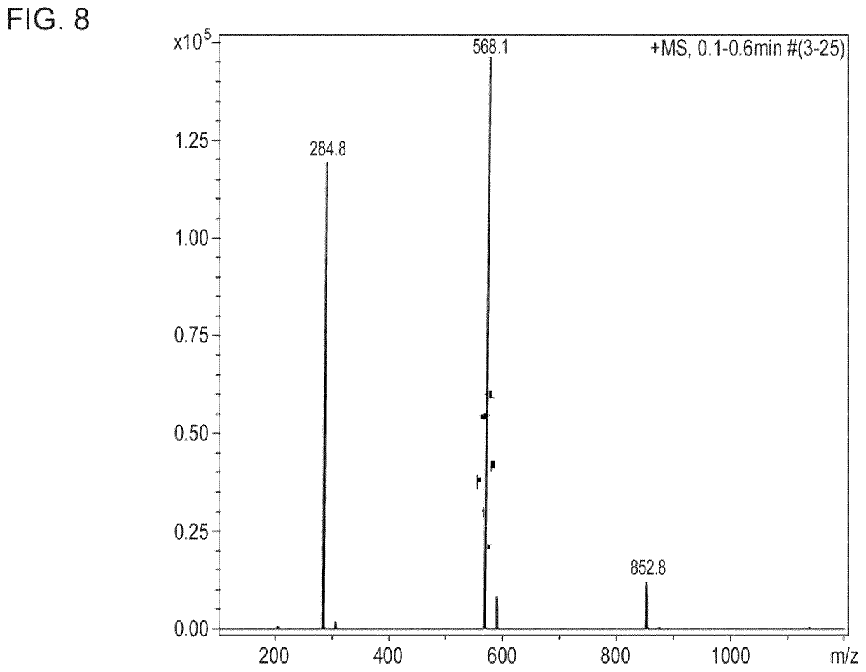

TABLE E

| Technique | Conclusions |
|---|---|
| Proton (¹H) and | Assignment of the proton (FIG. 5) and carbon |
| Carbon (¹³C) NMR | spectra (FIG. 6) are concordant with Psilocybin. |
| FT-Infrared | Assignment of the FT-IR spectrum (FIG. 7) is |
| Spectroscopy (FT-IR) | concordant with Psilocybin. |
| Mass Spectroscopy | Assignment of the mass spectrum (FIG. 8) is |
| (MS) | concordant with Psilocybin. |

Alternatively, and independently, the crystalline psilocybin may take the form of Hydrate A or Polymorph B.

In some embodiments, the disclosure provides the crystalline psilocybin in the form Polymorph A or Polymorph A′ for use in medicine. In some embodiments, the disclosure provides crystalline psilocybin Polymorph A for use in medicine. In some embodiments, the disclosure provides crystalline psilocybin Polymorph A′ for use in medicine. In some embodiments, the disclosure provides a high purity crystalline psilocybin Polymorph A for use in medicine. In some embodiments, the disclosure provides a high purity crystalline psilocybin Polymorph A′ for use in medicine. Alternatively, and independently, the crystalline psilocybin may take the form of Hydrate A or Polymorph B.

In some embodiments, the disclosure provides crystalline psilocybin in the form Polymorph A or Polymorph A′ for use in treating a subject in need thereof. Alternatively, and independently, the crystalline psilocybin may take the form of Hydrate A or Polymorph B.

In some embodiments, the disclosure provides crystalline psilocybin, Polymorph A or Polymorph A′, for use in treating a subject in need thereof. In some embodiments, the disclosure provides crystalline psilocybin, Polymorph A or Polymorph A′, for use in treating a subject in need thereof. In some embodiments, the disclosure provides crystalline psilocybin Polymorph A for use in treating a subject in need thereof. In some embodiments, the disclosure provides crystalline psilocybin Polymorph A′ for use in treating a subject in need thereof. In some embodiments, the disclosure provides a high purity crystalline psilocybin Polymorph A for use in treating a subject in need thereof. In some embodiments, the disclosure provides a high purity crystalline psilocybin Polymorph A′ for use in treating a subject in need thereof.

### Pharmaceutical Compositions and Formulations

In some embodiments, the disclosure provides a pharmaceutical composition comprising crystalline psilocybin and one or more pharmaceutically acceptable carriers or excipients.

In some embodiments, the disclosure provides a pharmaceutical formulation comprising high purity psilocybin and one or more pharmaceutically acceptable carriers or excipients. In some embodiments, the disclosure provides a pharmaceutical formulation comprising crystalline psilocybin Polymorph A and one or more pharmaceutically acceptable carriers or excipients. In some embodiments, the disclosure provides a pharmaceutical formulation comprising crystalline psilocybin Polymorph A′ and one or more pharmaceutically carriers or excipients. In some embodiments, the disclosure provides a pharmaceutical formulation comprising high purity crystalline psilocybin, Polymorph A or Polymorph A′, and one or more pharmaceutically acceptable carriers or excipients. In some embodiments, the disclosure provides a pharmaceutical formulation comprising high purity crystalline psilocybin Polymorph A and one or more pharmaceutically acceptable carriers or excipients. In some embodiments, the disclosure provides a pharmaceutical formulation comprising high purity crystalline psilocybin Polymorph A′ and one or more pharmaceutically acceptable carriers or excipients.

In some embodiments, pharmaceutical excipients for an oral formulation include: diluents, such as microcrystalline cellulose, starch (e.g., pregelatinized starch or partially pregelatinized starch), mannitol, calcium hydrogen phosphate anhydrous or co-mixtures of silicon dioxide, calcium carbonate, microcrystalline cellulose and talc; disintegrants, such as sodium starch glycolate or croscarmellose sodium; binders, such as povidone, co-povidone or hydroxyl propyl cellulose; lubricants, such as magnesium stearate or sodium stearyl fumurate; glidants, such as colloidal silicon dioxide; and film coats, such as Opadry II white or PVA based brown Opadry II.

In some embodiments, the oral dosage form also comprises a disintegrant, such as, but not limited to: starch glycolate, croscarmellose sodium, and/or mixtures thereof. In some embodiments, the oral dosage form comprises 3% or less by wt disintegrant, less than 3% by wt disintegrant and greater than 0.001% by wt disintegrant, about 2.5% by wt or less disintegrant; 2% by wt or less disintegrant; 1.5% by wt or less disintegrant; 1% by wt or less disintegrant; 0.7% by wt or less disintegrant; 0.5% by wt or less disintegrant, or 0.3% by wt or less disintegrant.

In some embodiments, the disintegrant is sodium starch glycolate. In some embodiments, the sodium starch glycolate is present at less than 3% wt. In Other embodiments, the sodium starch glycolate is present at about 2% by wt or less, about 2% by wt; about 1% by wt or less, about 1% by wt; about 0.7% by wt or less, about 0.7% by wt; about 0.5% by wt or less, or about 0.5% by wt. In still other embodiments, the sodium starch glycolate is present at about 0.5% to 1% by wt.

In some embodiments, the oral dosage form comprises 5 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 10 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 25 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5%.

In some embodiments, a pharmaceutical composition or formulation described herein comprises psilocybin, pergelatanized starch, and sodium stearyl fumarate. In some embodiments, the pharmaceutical composition or formulation comprises about 1%-10%, by weight, psilocybin. In some embodiments, the pharmaceutical composition or formulation comprises about 85-99%, by weight, pregelatinized starch. In some embodiments, the pharmaceutical composition or formulation comprises about 0.5%-2%, by weight, sodium stearyl fumarate.

In embodiments, a pharmaceutical composition or formulation described herein comprises (a) about 1%-10%, by weight, psilocybin; (b) about 85-99%, by weight, pregelatinized starch; and (c) about 0.5%-2%, by weight, sodium stearyl fumarate. In some embodiments, the pharmaceutical composition or formulation is one or more pharmaceutical compositions or formulations described in WIPO Patent Appln. Pub. No. 2022/207746 and 2019/073379, the entire contents of which are hereby incorporated by reference.

In some embodiments, the pharmaceutical formulation is a parenteral dosage form. In some embodiments, the pharmaceutical formulation is an oral dosage form. In some embodiments, the pharmaceutical composition comprises a tablet. In some embodiments, the pharmaceutical composition comprises a capsule. In some embodiments, the pharmaceutical composition comprises a dry powder. In some embodiments, the pharmaceutical composition comprises a solution. In some embodiments, more than one dosage form is administered to the subject at substantially the same time. In some embodiments, the subject may be administered the entire therapeutic dose in one tablet or capsule. In some embodiments, the therapeutic dose may be split among multiple tablets or capsules. For example, for a dose of 25 mg, the subject may be administered 5 tablets or capsules each comprising 25 mg of psilocybin. Alternatively, for a dose of 10 mg, the subject may be administered 2 tablets or capsules each comprising 5 mg of psilocybin.

In some embodiments, the oral dosage form comprises a functional filler. The functional filler may be a silicified filler, such as, but not limited to silicified microcrystalline cellulose (SMCC). In some embodiments, the oral dosage form comprises high compactability grades of SMCC with a particle size range of from about 45 to 150 microns. A mixture of two functional fillers having different particle size ranges may be used with the weight percentages of the two favoring the larger sized particles.

In some embodiments, the silicified microcrystalline filler may comprise a first filler, having a particle size range of from about 45 to 80 microns in an amount of up to 30%, up to 20%, up to 15%, or less by weight of filler, and a second filler, having a particle size range of from about 90 to 150 microns, in an amount of up to 70%, up to 80%, up to 85%, or more, by weight of filler.

In some embodiments, the oral dosage form may comprise silicified microcrystalline cellulose with a particle size range of from about 45 to 80 microns (SMCC 50), such as Prosolv 50; silicified microcrystalline cellulose with a particle size range of from about 90 to 150 microns (SMCC 90), such as Prosolv 90; or mixtures thereof. In other embodiments, the oral dosage form may comprise SMCC 50 and SMCC 90. In other embodiments, the oral dosage form may comprise SMCC 50 and SMCC 90, wherein the ratio of SMCC 50 to SMCC 90 is 1:5 to 1:8 wt %. In still other embodiments the ratio of SMCC 50 to SMCC 90 is 1:5-1:7; 1:6-1:7; 1:6-1:8; or 1.7-1.8. In still other embodiments the ratio of SMCC 50 to SMCC 90 is 1:6; 1:6.1; 1:6.2; 1:6.3; 1:6.4; 1:6.5; 1:6.6; 1.6.7; 1:6.8; 1.6.9; or 1:7. The formulation may further comprise or consist essentially of a disintegrant, including without limitation sodium starch glycolate; a glidant, including without limitation colloidal silicon dioxide; and a lubricant, including without limitation sodium stearyl fumarate.

In some embodiments, the oral dosage form may comprise a disintegrant such as sodium starch glycolate, at less than 3% (by wt), less than 2%, or 1% or less.

In some embodiments, the oral dosage form comprises 5 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 10 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 25 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 5 mg of crystalline psilocybin in the form of Polymorph A, 12.5 mg of SMCC 50, 79.5 mg of SMCC 90, 1 mg sodium starch glycolate, 1 mg colloidal silicon dioxide and 1 mg sodium stearyl fumarate. In some embodiments, the tablet or capsule comprises 5 mg of crystalline psilocybin in the form of Polymorph A, 12.5 mg of SMCC 50, 79.5 mg of SMCC 90, 1 mg sodium starch glycolate, 1 mg colloidal silicon dioxide and 1 mg sodium stearyl fumarate.

In some embodiments, the oral dosage form comprises 1 mg of crystalline psilocybin in the form of Polymorph A, 20.5 mg of SMCC 50, 75.5 mg of SMCC 90, 1 mg sodium starch glycolate, 1 mg colloidal silicon dioxide, and 1 mg sodium stearyl fumarate. In some embodiments, the tablet or capsule comprises 1 mg of crystalline psilocybin in the form of Polymorph A, 20.5 mg of SMCC 50, 75.5 mg of SMCC 90, 1 mg sodium starch glycolate, 1 mg colloidal silicon dioxide, and 1 mg sodium stearyl fumarate.

In some embodiments, the tablet or capsule comprises one or more excipients. Non-limiting exemplary excipients include microcrystalline cellulose and starch, including without limitation silicified microcrystalline cellulose.

It should be noted that the formulations may comprise psilocybin in any form, not only the polymorphic forms disclosed herein.

As used herein, oral doses of psilocybin are classified follows: “very low doses” (about 0.045 mg/kg or less); “low doses” (between about 0.115 and about 0.125 mg/kg), “medium doses” (between about 0.115 to about 0.260 mg/kg), and “high doses” (about 0.315 mg/kg or more). See Studerus et al (2011) J Psychopharmacol 25 (11) 1434-1452.

In some embodiments, the formulated dose of psilocybin comprises from about 0.01 mg/kg to about 1 mg/kg. In some embodiments, a human dose (for an adult weighing 60-80 kg) comprises between about 0.60 mg and about 80 mg.

In some embodiments, a formulated dose comprises between about 2 and about 50 mg of crystalline psilocybin. In some embodiments, a formulated dose comprises between 2 and 40 mg, between 2 and 10 mg, between 5 and 30 mg, between 5 and 15 mg, or between 20 and 30 mg of crystalline psilocybin. In some embodiments, a formulated dose comprises about 1 mg, about 5 mg, about 10 mg, or about 25 mg of crystalline psilocybin.

In some embodiments, a formulated dose comprises between about 2 and about 50 mg of crystalline psilocybin Polymorph A or Polymorph A′ or a mixture thereof. In some embodiments, a formulated dose comprises between 2 and 40 mg, between 2 and 10 mg, between 5 and 30 mg, between 5 and 15 mg, or between 20 and 30 mg of crystalline psilocybin Polymorph A or Polymorph A′ or a mixture thereof. In some embodiments, a formulated dose comprises about 1 mg, about 5 mg, about 10 mg, or about 25 mg of crystalline psilocybin Polymorph A or Polymorph A′ or a mixture thereof. In some embodiments, a formulated dose comprises about 5 mg of crystalline psilocybin Polymorph A or Polymorph A′ or a mixture thereof.

In some embodiments, a formulated dose comprises between about 2 and about 50 mg of crystalline psilocybin Polymorph A. In some embodiments, a formulated dose comprises between 2 and 40 mg, between 2 and 10 mg, between 5 and 30 mg, between 5 and 15 mg, or between 20 and 30 mg of crystalline psilocybin Polymorph A. In some embodiments, a formulated dose comprises about 1 mg, about 5 mg, about 10 mg, or about 25 mg of crystalline psilocybin Polymorph A.

In some embodiments, a formulated dose comprises between about 2 and about 50 mg of crystalline psilocybin Polymorph A′. In some embodiments, a formulated dose comprises between 2 and 40 mg, between 2 and 10 mg, between 5 and 30 mg, between 5 and 15 mg, or between 20 and 30 mg of crystalline psilocybin Polymorph A′. In some embodiments, a formulated dose comprises about 1 mg, about 5 mg, about 10 mg, or about 25 mg of crystalline psilocybin Polymorph A′.

In some embodiments, a formulated dose comprises between about 2 and about 50 mg of crystalline psilocybin Polymorph B. In some embodiments, a formulated dose comprises between 2 and 40 mg, between 2 and 10 mg, between 5 and 30 mg, between 5 and 15 mg, or between 20 and 30 mg of crystalline psilocybin Polymorph B. In some embodiments, a formulated dose comprises about 1 mg, about 5 mg, about 10 mg, or about 25 mg of crystalline psilocybin Polymorph B.

In some embodiments, a formulated dose comprises between about 2 and about 50 mg of crystalline psilocybin Hydrate A. In some embodiments, a formulated dose comprises between 2 and 40 mg, between 2 and 10 mg, between 5 and 30 mg, between 5 and 15 mg, or between 20 and 30 mg of crystalline psilocybin Hydrate A. In some embodiments, a formulated dose comprises about 1 mg, about 5 mg, about 10 mg, or about 25 mg of crystalline psilocybin Hydrate A.

### Dosing

In some embodiments, a therapeutically effective dose of psilocybin is administered to the subject. In some embodiments, each dose of psilocybin administered to the subject is a therapeutically effective dose.

In some embodiments, a dose of psilocybin may be in the range of about 1 mg to about 100 mg. For example, the dose may be about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, the dose of psilocybin is between about 0.1 mg to about 100 mg, about 1 mg to about 50 mg, or about 5 mg to about 30 mg. In some embodiments, the dose of psilocybin is about 1 mg, about 10 mg, or about 25 mg. In some embodiments, the dose of psilocybin is in the range of about 0.001 mg to about 1 mg. In some embodiments, the dose of psilocybin is in the rage of about 100 mg to about 250 mg. In some embodiments, the dose of psilocybin is about 25 mg. In some embodiments, the psilocybin is in the form of polymorph A.

In some embodiments, an adult oral dose comprises about 1 mg to about 40 mg, about 2 to about 30 mg, or about 15 to about 30 mg of crystalline psilocybin, for example about 1 mg, about 5 mg, about 10 mg, or about 25 mg of crystalline psilocybin. In some embodiments, an adult oral dose comprises about 25 mg of crystalline psilocybin. In some embodiments, the crystalline psilocybin is in the form of polymorph A.

In some embodiments, a “micro-dose” of psilocybin is administered to a subject. A micro-dose may comprise, for example, about 0.05 mg to about 2.5 mg of crystalline psilocybin, such as about 1.0 mg. In the case of micro-dosing the regime may comprise a regular, continuous regime of, for example, daily administration, every other day administration, or weekly, administration. Such dosing may be absent of psychological support.

In some embodiments, one dose of psilocybin is administered to the subject. In some embodiments, multiple doses of psilocybin are administered to the subject. For example, at least 2, at least 3, at least 4, at least 5, at least 6, at least 7, at least 8, at least 9, at least 10, at least 15, at least 20, at least 25, at least 30, or at least 50 doses of psilocybin may be administered to the subject. In some embodiments, the same dose of psilocybin is administered to a subject during each administration. In some embodiments, a different dose of psilocybin is administered to a subject during each administration. In some embodiments, the dose of psilocybin administered to the subject is increased over time. In some embodiments, the dose of psilocybin administered to the subject is decreased over time.

In some embodiments, the psilocybin is administered at therapeutically effective intervals. In some embodiments, a therapeutically effective interval may be about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 29 weeks, or about 30 weeks, including all values and ranges therein. In some embodiments, a therapeutically effective interval may be about 20 weeks to about 30 weeks, or about 25 to about 30 weeks, or about 25-26 weeks, or about 26 weeks. In some embodiments, a therapeutically effective interval (e.g., for a patient that responded to a first dose of psilocybin) may be about 20 weeks to about 30 weeks, or about 25 to about 30 weeks, or about 25-26 weeks, or about 26 weeks. In some embodiments, a therapeutically effective interval may be about 1 month, about 3 months, about 6 months, or about 12 months. In some embodiments, the psilocybin is administered once per day. In some embodiments, the psilocybin is administered at least once per week or at least twice per week. In some embodiments, the psilocybin is administered at least once per month or at least twice per month. In some embodiments, the psilocybin is administered at least once every three months, at least once every six months, or at least once every 12 months.

In some embodiments, a first dose and a second dose of psilocybin are administered to the subject. In some embodiments, the first dose is about 1 mg and the second dose is about 1 mg. In some embodiments, the first dose is about 10 mg and the second dose is about 10 mg. In some embodiments, the first dose is about 25 mg and the second dose is about 25 mg. In some embodiments, the first dose is about 10 mg and the second dose is about 25 mg. In some embodiments, the first dose is about 25 mg and the second dose is about 10 mg. In some embodiments, the first dose is about 1 mg and the second dose is about 10 mg. In some embodiments, the first dose is about 1 mg and the second dose is about 25 mg. In some embodiments, the first dose is about 10 mg and the second dose is about 1 mg. In some embodiments, the first dose is about 25 mg and the second dose is about 1 mg.

In some embodiments a second dose of psilocybin is administered from about one week to about 12 weeks after a first dose. In some embodiments, a second dose of psilocybin is administered about one week after a first dose. In some embodiments, a second dose of psilocybin is administered about two weeks after a first dose. In some embodiments, a second dose of psilocybin is administered about three weeks after a first dose. In some embodiments, a second dose of psilocybin is administered about four weeks after a first dose. In some embodiments, a second dose of psilocybin is administered about five weeks after a first dose. In some embodiments, a second dose of psilocybin is administered about six weeks after a first dose.

### Administration Routes

Exemplary modes for administration of psilocybin include oral, parenteral (e.g., intravenous, subcutaneous, intradermal, intramuscular [including administration to skeletal, diaphragm and/or cardiac muscle], intradermal, intrapleural, intracerebral, and intra-articular), topical (e.g., to both skin and mucosal surfaces, including airway surfaces, and transdermal administration), inhalation (e.g., via an aerosol), rectal (e.g., via a suppository), transmucosal, intranasal, buccal (e.g., sublingual), vaginal, intrathecal, intraocular, transdermal, in utero (or in ovo), intralymphatic, and direct tissue or organ injection (e.g., to liver, skeletal muscle, cardiac muscle, diaphragm muscle or brain). In some embodiments, psilocybin is administered orally to the subject.

### Methods of Treatment

In some embodiments, the methods provide herein relate to treating treatment-resistant depression in a subject in need thereof.

Re-Dosing Subjects that Did not Respond to a First Dose of Psilocybin

In some embodiments, provided herein are methods of treating treatment-resistant depression in a subject or patient population that do not respond to a first dose of psilocybin (such subject or patient population may be referred to herein as a “non-responder”). Without being bound to any particular theory, it is contemplated that administering a second dose of psilocybin to the subject or patient population can elicit a therapeutic response in the subjects or patient population. The second dose or “re-dosing” can occur after a subject is identified as a non-responder to the first dose of psilocybin, e.g., from 1 day to about 3 weeks after administration of the first dose. In some embodiments, the second dose of psilocybin is administered about 3 weeks after administration of the first dose.

In some embodiments, the second dose of psilocybin is administered on Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, Day 20, or Day 21 after administering the first dose of psilocybin. In some embodiments, the second dose of psilocybin is administered about 1 week, about 1.5 weeks, about 2 weeks, about 2.5 weeks, about 3 weeks, about 3.5 weeks, about 4 weeks, about 4.5 weeks, about 5 weeks, about 5.5 weeks, or about 6 weeks after administering the first dose of psilocybin. As used herein, phrases such as “Day 2”, and the like refer to the number of days after administering the first dose of psilocybin according to the methods of the disclosure. That is, Day 0 is the date on which the first dose of psilocybin is administered. Day 1 is the first day after receiving the first dose of psilocybin. Day 2 is the second day, and so on. Administering the first dose of psilocybin can refer to the first administration to a psilocybin-naive patient who has never been administered psilocybin, or to a patient who may have been administered psilocybin in the past, but has ceased treatment with psilocybin for a period of time sufficient to require re-introduction.

In some embodiments, the subject exhibits no or substantially no reduction in symptoms of depression after administering the first dose of psilocybin. For example, the subject experiences no or substantially no reduction in, but not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

In some embodiments, the methods comprise measuring the subject's depressive symptoms using a clinical depression evaluation (as described herein). In some embodiments, the clinical depression evaluation includes a rating scale (e.g., Montgomery-Åsberg Depression Rating Scale (MADRS)). In some embodiments, the clinical depression evaluation includes using an observational study (described herein) to assess whether the patient exhibits depressive symptoms.

In some embodiments, the subject has a MADRS of 7 to 34 at baseline (i.e., before administration of the first dose of psilocybin). In some embodiments, the subject has a MADRS score of 7, 8, 9, 10, 11, 12, 13, 14, 15, 16, 17, 18, 19, 20, 21, 22, 23, 24, 25, 26, 27, 28, 29, 30, 31, 32, 33, or 34 at baseline. In some embodiments, the subject has a MADRS score of greater than 34 at baseline. In some embodiments, the subject has mild depression (MADRS of 7 to 19) at baseline and/or after the first dose of psilocybin. In some embodiments, the subject has moderate depression (MADRS of 20 to 34) at baseline and/or after the first dose of psilocybin. In some embodiments, the subject has severe depression (MADRS of greater than 34) at baseline and/or after the first dose of psilocybin.

In some embodiments, the subject that is non-responsive has a MADRS of 7 to 34 after administration of a first dose of psilocybin. In some embodiments, the subject has a MADRS of 7, 8, 9, 10, 11, 12, 13, 14, 15, 16, 17, 18, 19, 20, 21, 22, 23, 24, 25, 26, 27, 28, 29, 30, 31, 32, 33, or 34 after administration of a first dose of psilocybin. In some embodiments, the subject has a MADRS of greater than 34 after administration of a first dose of psilocybin.

In some embodiments, the subject that is non-responsive has a change in baseline in a MADRS of about-10 to 0 after administering the first dose of psilocybin. In some embodiments, the subject has a change in baseline in the MADRS of −10, −9, −8, −7, −6, −5, −4, −3, −3, −1, or 0 (or the MADRS increases, indication worsening of depression) after administering the first dose of psilocybin. In some embodiments, the subject that is non-responsive has a change in baseline in a MARDS of less than 50% after administering the first dose of psilocybin. In some embodiments, the subject that is non-responsive has a change in baseline in a MARDS of 45%, 40%, 35%, 30%, 25%, 20%, 15%, 10%, 5% or less after administering the first dose of psilocybin.

In some embodiments, the methods comprise determining the change in baseline in the MADRS on Day 1, Day 2, Day 3, Day 4, Day 5, or Day 6 after administering the first dose of psilocybin. In some embodiments, the change in baseline in the MADRS is determined on Day 1. In some embodiments, the methods comprise determining the MADRS on Day 2 after administering the first dose of psilocybin. In some embodiments, comprise determining the MADRS on Day 3 after administering the first dose of psilocybin. In some embodiments, comprise determining the MADRS on Day 4 after administering the first dose of psilocybin. In some embodiments, comprise determining the MADRS on Day 5 after administering the first dose of psilocybin. In some embodiments, comprise determining the MADRS on Day 6 after administering the first dose of psilocybin. In some embodiments, the methods comprise determining the change in baseline in the MADRS on Day 2, Day 3, Day 4, Day 5, and Day 6 after administering the first dose of psilocybin.

In some embodiments, the methods comprise determining the change in baseline in the MADRS 1 week, 2 weeks, or 3 weeks after administering the first dose of psilocybin. In some embodiments, the methods comprise determining the change in baseline in the MADRS 1 week after administering the first dose of psilocybin. In some embodiments, the methods comprise determining the change in baseline in the MADRS 2 weeks after administering the first dose of psilocybin. In some embodiments, the methods comprise determining the change in baseline in the MADRS 3 weeks after administering the first dose of psilocybin. In some embodiments, the methods comprise determining the change in baseline in the MADRS 1 week, 2 weeks, and 3 weeks after administering the first dose of psilocybin.

In some embodiments, the subject that did not respond to the first dose of psilocybin, is responsive to a second dose of psilocybin. In some embodiments, the subject that did not respond to the first dose of psilocybin, exhibits a reduction in symptoms of depression after administering a second dose of psilocybin. In some embodiments, the methods comprising identifying the subject as responsive to the second dose of psilocybin using a clinical depression evaluation described herein, e.g., a clinical rating scale. In some embodiments, the clinical rating scale is MADRS. In some embodiments, the subject that did not response to the first dose of psilocybin has a change in baseline in a MADRS of −11 to −30 after administration of the second dose of psilocybin. In some embodiments, the subject has a change in baseline in a MADRS of −11, −12, −13, −14, −15, −16, −17, −18, −19, −20, −21, −22, −23, −24, −25, −26, −27, −28, −29, or −30 after administration of the second dose of psilocybin. In some embodiments, the subject that is non-responsive to the first dose of psilocybin has a change in baseline in MARDS of greater than 50% after administering the second dose of psilocybin. In some embodiments, the subject that is non-responsive to the first dose of psilocybin has a change in baseline in MARDS of 50%, 55%, 60%, 65%, 70%, 75%, 80%, 85%, 90%, 95%, or 100% after administering the first dose of psilocybin.

In some embodiments, the methods comprise determining the change in baseline in the MADRS on Day 1, Day 2, Day 3, Day 4, Day 5, or Day 6 after administering the second dose of psilocybin for the non-responder. In some embodiments, the methods comprise determining the change in baseline in the MADRS on Day 1 after administering the second dose of psilocybin. In some embodiments, the methods comprise determining the change in baseline in the MADRS on Day 2 after administering the second dose of psilocybin. In some embodiments, the methods comprise determining the change in baseline in the MADRS on Day 3 after administering the second dose of psilocybin. In some embodiments, the methods comprise determining the change in baseline in the MADRS on Day 4 after administering the second dose of psilocybin. In some embodiments, the methods comprise determining the change in baseline in the MADRS on Day 5 after administering the second dose of psilocybin. In some embodiments, the methods comprise determining the change in baseline in the MADRS on Day 6 after administering the second dose of psilocybin. In some embodiments, the methods comprise determining the change in baseline in the MADRS on Day 2, Day 3, Day 4, Day 5, and Day 6 after administering the second dose of psilocybin.

In some embodiments, the methods comprise determining the change in baseline in the MADRS 1 week, 2 weeks, or 3 weeks after administering the second dose of psilocybin in the non-responder. In some embodiments, the methods comprise determining the change in baseline in the MADRS 1 week after administering the second dose of psilocybin. In some embodiments, the methods comprise determining the change in baseline in the MADRS 2 weeks after administering the second dose of psilocybin. In some embodiments, the methods comprise determining the change in baseline in the MADRS 3 weeks after administering the second dose of psilocybin. In some embodiments, the methods comprise determining the change in baseline in the MADRS 1 week, 2 weeks, and 3 weeks after administering the second dose of psilocybin.

In some embodiments, the subject has a MADRS of 0 to 10 after administration of a second dose of psilocybin (e.g., 0, 1, 2, 3, 4, 5, 6, 7, 8, 9, or 10). In some embodiments, the subject has a MADRS of 0 to 6 after administration of a second dose of psilocybin. In some embodiments, the subject has a MADRS of 0, 1, 2, 3, 4, 5, or 6 after administration of a second dose of psilocybin.

In some embodiments, the subject has no symptoms of depression (MADRS 0 to 6) after administration of the second dose of psilocybin. In some embodiments, the non-responder has severe depression at baseline and/or after the first dose of psilocybin, and then after the second dose of psilocybin the subject has moderate, mild or no depression. In some embodiments, the subject has moderate depression at baseline and/or after the first dose of psilocybin, and then after the second dose of psilocybin the subject has mild or no depression. In some embodiments, the subject has mild depression at baseline and/or after the first dose of psilocybin, and then after the second dose of psilocybin the subject has no depression.

In some embodiments, the methods include treating treatment-resistant depression in a subject in need thereof, comprising administering at least two doses of psilocybin to the subject, wherein the subject does not respond to the first dose of psilocybin, and wherein the second dose of psilocybin is administered about 3 weeks after administering the first dose.

In some embodiments, the methods include treating treatment-resistant depression in a subject in need thereof, comprising administering at least two doses of psilocybin to the subject, wherein the subject does not respond to the first dose of psilocybin, wherein the second dose of psilocybin is administered about 3 weeks after administering the first dose, and wherein the first dose comprises 25 mg of psilocybin and the second dose comprises 25 mg of psilocybin.

In some embodiments, the methods include treating treatment-resistant depression with psilocybin in a subject that did not response to a first dose of psilocybin, comprising administering a second dose of psilocybin 3 weeks after administering the first dose.

In some embodiments, the methods include treating treatment-resistant depression with psilocybin in a subject that did not response to a first dose of psilocybin, comprising administering a second dose of psilocybin 3 weeks after administering the first dose, wherein the first dose comprises 25 mg of psilocybin and the second dose comprises 25 mg of psilocybin.

In some embodiments, the methods include treating treatment-resistant depression in a subject in need thereof, comprising administering a first dose of psilocybin to the subject; measuring the subject's depressive symptoms using a clinical depression evaluation after the first dose of psilocybin; identifying the subject as a non-responder to the first dose of psilocybin; and then administering a second dose of psilocybin to the subject 3 weeks after administering the first dose.

In some embodiments, the methods include treating treatment-resistant depression in a subject in need thereof, comprising administering a first dose of psilocybin to the subject; measuring the subject's depressive symptoms using a clinical depression evaluation after the first dose of psilocybin; identifying the subject as a non-responder to the first dose of psilocybin; and administering a second dose of psilocybin to the subject 3 weeks after administering the first dose, wherein the first dose comprises 25 mg of psilocybin and the second dose comprises 25 mg of psilocybin.

In some embodiments, the methods include treating treatment-resistant depression in a subject in need thereof, comprising administering a first dose of psilocybin to the subject; measuring the subject's depressive symptoms using a MADRS after the first dose of psilocybin; identifying the subject as a non-responder to the first dose of psilocybin; and administering a second dose of psilocybin to the subject 3 weeks after administering the first dose.

In some embodiments, the methods include treating treatment-resistant depression in a subject in need thereof, comprising administering a first dose of psilocybin to the subject; measuring the subject's depressive symptoms using a MADRS after the first dose of psilocybin; identifying the subject as a non-responder to the first dose of psilocybin; and administering a second dose of psilocybin to the subject 3 weeks after administering the first dose, wherein the first dose comprises 25 mg of psilocybin and the second dose comprises 25 mg of psilocybin.

Re-Dosing Subjects that Responded to a First Dose of Psilocybin

Provided herein are methods of treating treatment-resistant depression in subjects or patient populations that respond to a first dose of psilocybin (“responders”). As shown in Tables 9-10, the median time to a first depressive event after is about 189 days (27 weeks) (24 days, 95% CI), suggesting that a first dose of psilocybin is effective out to at least about 27 weeks after administration. Without being bound to any particular theory, it is suggested that administration of second dose of psilocybin prior to the median time to the first depressive event can prevent the occurrence of a next depressive event.

In some embodiments, the time to the first depressive event after the first dose of psilocybin (e.g. in a patient that responded to the first dose of psilocybin) is about 100 days, about 110 days, about 120 days, about 130 days, about 140 days, about 150 days, about 160 days, about 170 days, about 180 days, about 190 days, about 200 days, about 210 days, about 220 days, about 230 days, about 240 days, or about 250 days. In some embodiments, the time to the first depressive event after the first dose of psilocybin is about 175 days, about 176 days, about 177 days, about 178 days, about 179 days, about 180 days, about 181 days, about 182 days, about 183 days, about 184 days, about 185 days, about 186 days, about 187 days, about 189 days, about 190 days, about 191 days, about 192 days, about 193 days, about 194 days, about 195 days, about 196 days, about 197 days, about 198 days, about 199 days, about 200 days, about 201 days, about 202 days, about 203 days, about 204 days, or about 205 days, including all values and ranges therein.

In some embodiments, the next depressive event comprises: initiation of new antidepressant treatment; hospitalization due to depression/suicidality; suicide attempt, prevention of an imminent suicide attempt, or completed suicide; increased suicidality measured by worsening on MADRS item 10; active suicidal ideation measured by the C-SSRS; MADRS worsening; or discontinuation for adverse event or lack of efficacy.

In some embodiments, the methods include treating treatment-resistant in a subject that responded to a first dose of psilocybin, comprising administering a second dose at least about 20 weeks after administering the first dose (e.g., about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks, or more).

In some embodiments, the methods include treating treatment-resistant in a subject that responded to a first dose of psilocybin, comprising administering a second dose at least about 26 weeks after administering the first dose.

In some embodiments, the methods include treating treatment-resistant in a subject that responded to a first dose of psilocybin, comprising administering a second dose about 20 to about 28 weeks after administering the first dose. In some embodiments, the second dose is administered at least about 20 weeks, about 21 weeks, about 22 weeks, about 23 weeks, about 24 weeks, about 25 weeks, about 26 weeks, about 27 weeks, or about 28 weeks.

In some embodiments, the subject that responded to the first dose of psilocybin experiences a reduction in symptoms of depression after administering the first dose of psilocybin. For example, the subject experiences a reduction in, but not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

In some embodiments, the methods further comprise measuring depressive symptoms after administering the first dose of psilocybin using a clinical depression evaluation described herein, e.g., a clinical depression rating scale. In some embodiments, the clinical depression rating scale is MADRS. In some embodiments, the subject that responded to the first dose of psilocybin has a change in baseline in the MADRS of −11 to −30 after administering the first dose pf psilocybin. In some embodiments, the subject has a change in baseline in the MADRS of −11, −12, −13, −14, −15, −16, −17, −18, −19, −20, −21, −22, −23, −24, −25, −26, −27, −28, −29, or −30 after administering the first dose of psilocybin.

In some embodiments, the methods comprise determining the change in baseline in the MADRS on Day 2, Day 3, Day 4, Day 5, or Day 6 after administering the first dose of psilocybin in the responder. In some embodiments, the methods comprise determining the change in baseline in the MADRS on Day 2 after administering the first dose of psilocybin. In some embodiments, the methods comprise determining the change in baseline in the MADRS on Day 3 after administering the first dose of psilocybin. In some embodiments, the methods comprise determining the change in baseline in the MADRS on Day 4 after administering the first dose of psilocybin. In some embodiments, the methods comprise determining the change in baseline in the MADRS on Day 5 after administering the first dose of psilocybin. In some embodiments, the methods comprise determining the change in baseline in the MADRS on Day 6 after administering the first dose of psilocybin. In some embodiments, the methods comprise determining the change in baseline in the MADRS on Day 2, Day 3, Day 4, Day 5, and Day 6 after administering the first dose of psilocybin.

In some embodiments, the methods comprise determining the change in baseline in the MADRS of 1 week, 5 weeks, 10 weeks, 12 weeks, 15 weeks, 16 weeks, 20 weeks, 24 weeks, 25 weeks, or 28 weeks after administering the first dose of psilocybin in the responder. In some embodiments, the methods comprise determining the change in baseline in the MADRS of 1 week after administering the first dose of psilocybin. In some embodiments, the methods comprise determining the change in baseline in the MADRS of 2 weeks after administering the first dose of psilocybin. In some embodiments, the methods comprise determining the change in baseline in the MADRS of 5 weeks after administering the first dose of psilocybin. In some embodiments, the methods comprise determining the change in baseline in the MADRS of 10 weeks after administering the first dose of psilocybin. In some embodiments, the methods comprise determining the change in baseline in the MADRS of 12 weeks after administering the first dose of psilocybin. In some embodiments, the methods comprise determining the change in baseline in the MADRS of 15 weeks after administering the first dose of psilocybin. In some embodiments, the methods comprise determining the change in baseline in the MADRS of 16 weeks after administering the first dose of psilocybin. In some embodiments, the methods comprise determining the change in baseline in the MADRS of 20 weeks after administering the first dose of psilocybin. In some embodiments, the methods comprise determining the change in baseline in the MADRS of 24 weeks after administering the first dose of psilocybin. In some embodiments, the methods comprise determining the change in baseline in the MADRS of 25 weeks after administering the first dose of psilocybin. In some embodiments, the methods comprise determining the change in baseline in the MADRS of 28 weeks after administering the first dose of psilocybin. In some embodiments, the methods comprise determining the change in baseline in the MADRS of 1 week, 5 weeks, 10 weeks, 12 weeks, 15 weeks, 16 weeks, 20 weeks, 24 weeks, 25 weeks, and 28 weeks after administering the first dose of psilocybin.

In some embodiments, the methods comprise determining the change in baseline in the MADRS up to 1 day before administering the second dose of psilocybin in the subject. In some embodiments, the methods comprise determining the change in baseline in the MADRS 1 week, 6 days, 5 days, 4 days, 3 days, 3 days, or 1 day before administering the second dose of psilocybin.

In some embodiments, the subject maintains a change in baseline in the MADRS of −11 to −30 after administering the second dose of psilocybin. In some embodiments, the subject maintains a change in baseline in the MADRS of −11, −12, −13, −14, −15, −16, −17, −18, −19, −20, −21, −22, −23, −24, −25, −26, −27, −28, −29, or −30 after administering the second dose.

In some embodiments, the subject has a MADRS of 7 to 34 at baseline (i.e., before administration of the first dose of psilocybin). In some embodiments, the responder has a MADRS score of 7, 8, 9, 10, 11, 12, 13, 14, 15, 16, 17, 18, 19, 20, 21, 22, 23, 24, 25, 26, 27, 28, 29, 30, 31, 32, 33, or 34 at baseline. In some embodiments, the responder has a MADRS score of greater than 34 at baseline.

In some embodiments, the subject has a MADRS of 7 to 25 after administration of a first dose of psilocybin. In some embodiments, the responder has a MADRS of 7, 8, 9, 10, 11, 12, 13, 14, 15, 16, 17, 18, 19, 20, 21, 22, 23, 24, or 25 after administration of a first dose of psilocybin.

In some embodiments, the subject has a MADRS of 0 to 6 after administration of a first dose of psilocybin. In some embodiments, the subject has a MADRS of 0, 1, 2, 3, 4, 5, or 6 after administration of a first dose of psilocybin.

In some embodiments, the subject has a MADRS of 0 to 6 after administration of a second dose of psilocybin. In some embodiments, the subject has a MADRS of 0, 1, 2, 3, 4, 5, or 6 after administration of a second dose of psilocybin.

In some embodiments, the subject maintains a MADRS of 0 to 6 between administration of a first and second dose of psilocybin. In some embodiments, the subject maintains a MADRS of 0, 1, 2, 3, 4, 5, or 6 between administration of a first and second dose of psilocybin.

In some embodiments, the subject has mild depression (MADRS of 7 to 19) at baseline. In some embodiments, the subject has moderate depression (MADRS of 20 to 34) at baseline. In some embodiments, the subject has severe depression (MADRS of greater than 34) at baseline.

In some embodiments, the subject has no symptoms of depression (MADRS 0 to 6) after administration of the first dose of psilocybin. In some embodiments, the subject has no symptoms of depression (MADRS 0 to 6) after administration of the second dose of psilocybin.

In some embodiments, the subject has severe depression at baseline, and then after the first dose of psilocybin and/or the second dose of psilocybin the subject has moderate, mild or no depression. In some embodiments, the subject has moderate depression at baseline, and then after the first and/or second dose of psilocybin the subject has mild or no depression. In some embodiments, the subject has mild depression at baseline, and then after the first dose of psilocybin and/or second dose of psilocybin the subject has no depression.

In some embodiments, the methods include treating treatment-resistant in a subject that responded to a first dose of psilocybin, comprising administering a second dose at least about 20 weeks after administering the first dose, wherein the first dose comprises 25 mg of psilocybin and the second dose comprises 25 mg of psilocybin.

In some embodiments, the methods include treating treatment-resistant in a subject that responded to a first dose of psilocybin, comprising administering a second dose at least about 26 weeks after administering the first dose, wherein the first dose comprises 25 mg of psilocybin and the second dose comprises 25 mg of psilocybin.

It is to be understood by one of skill in the art that the methods of treatment comprising administering psilocybin, a prodrug of psilocybin, a metabolite of psilocybin, and/or a prodrug of a metabolite of psilocybin for the treatment of one or more indications as described herein also include: the use of psilocybin, a prodrug of psilocybin, a metabolite of psilocybin, and/or a prodrug of a metabolite of psilocybin in the manufacture of a medicament for the treatment of one or more indications as described herein; and the use of psilocybin, a prodrug of psilocybin, a metabolite of psilocybin, and/or a prodrug of a metabolite of psilocybin for the treatment of one or more indications as described herein.

Methods of treating various conditions using psilocybin are described in International Application Nos. PCT/IB2020/053687 and PCT/IB2020/053684, the contents of which are hereby incorporated by reference for all purposes. Methods of using benzodiazepines to increase sensitivity to psilocybin following a chronic SSRI regimen are described in International Application No. PCT/EP2021/079287, the contents of which are hereby incorporated by reference for all purposes.

In some embodiments, a method for treating a subject in need thereof comprises administering to the subject a therapeutically effective dose of psilocybin. In some embodiments, a method for treating a subject in need thereof comprises administering to the subject a therapeutically effective dose of psilocybin in a controlled environment, wherein the subject is provided with psychological support.

In some embodiments, a method for treating a subject in need thereof comprises at least one of the following:
(i) administering to the subject a therapeutically effective dose of psilocybin in a controlled environment, wherein the subject is provided with psychological support;
(ii) having the subject participate in one or more pre-administration psychological support session(s); and/or
(iii) having the subject participate in one or more post-administration psychological support session(s).

After administration of the psilocybin, the subject may not feel the effects of the drug for about 30 minutes to about 90 minutes. In some embodiments, the subject may not feel the effects of the drug for about 60 minutes. This period after administration and before the onset of effects will be referred to herein as the initial stage of the psilocybin session. The time marked by the onset of the drug's effects will be referred to herein as the early stage of the psilocybin session.

In some embodiments, the subject will experience the peak of the psilocybin's effects at about 1.5 hours to about 3.5 hours after administration thereof. The time period marked by the peak psilocybin experience will be referred to herein as the peak stage of the psilocybin session.

In some embodiments, the effects of the psilocybin may substantially wear off from about 4 hours to about 6 hours after administration. This time period will be referred to as the late stage of the psilocybin session.

In some embodiments, the subject's ability to reach a non-dual state (e.g., a mystical experience), or a sense of unity, boundlessness, ego-dissolution or transcendence correlates with positive clinical outcome. Each of these terms may be commonly defined as the breakdown of the usual relationship between self and other, whereby the subject might feel a oneness and increased sense of connectedness to the surrounding environment and/or the world at large.

In some embodiments, low levels of emotional arousal-which could indicate avoidance, lack of involvement or intellectualization-might, in some embodiments, be correlated with little or no improvement in treatment outcomes.

Factors that may influence the subjective experience of psilocybin include, for example, (i) dose, (ii) the mindset of the participant prior to the session, (iii) the setting of the session, (iv) the subject's ability to focus and stay with the experience, and/or (v) the subject's prior experience with psychedelics. These, and other factors, will be described in more detail below, along with ways to maximize therapeutic benefit of the psilocybin session.

### Pre-Administration Psychological Support Sessions

In some embodiments, the subject participates in at least one psychological support session before administration of the psilocybin (“pre-administration psychological support session”). In some embodiments, a pre-administration psychological support session may be held about 1 month prior to the psilocybin administration. In some embodiments, a pre-administration psychological support session may be held about 2 weeks prior to the psilocybin administration. In some embodiments, a pre-administration psychological support session may be held about 1 week prior to the psilocybin administration. In some embodiments, a pre-administration psychological support session may be held about 3 days prior to the psilocybin administration. In some embodiments, a pre-administration psychological support session may be held about 1 day prior to the psilocybin administration. In some embodiments, a pre-administration psychological support session may be held on the same day as and prior to psilocybin administration.

In some embodiments, the subject may participate in one, two, three, four, five, six, seven, or eight pre-administration psychological support sessions. In some embodiments, the subject may participate in at least two pre-administration psychological support sessions. In some embodiments, the subject may participate in at least three pre-administration psychological support sessions. In some embodiments, the subject may participate in pre-administration psychological support sessions at least once per week, for at least two or three weeks prior to the psilocybin session. In some embodiments, the subject may additionally participate in a pre-administration psychological support session the day before the psilocybin session.

The pre-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the pre-administration psychological support session(s).

In some embodiments, the goals of the pre-administration session may include (i) establishing therapeutic alliance between subject and therapist; (ii) answering the subject's questions and addressing any concerns; and/or (iii) demonstrating and practicing the skills of self-directed inquiry and experiential processing. In some embodiments, the pre-administration psychological support sessions focus on discussion of possible psilocybin effects, and/or preparing subjects for the dosing session by practicing relevant therapeutic techniques to reduce avoidance and anxiety, eliciting relevant therapeutic goals, building rapport, and/or establishing therapeutic alliance. During the psychological support session, skills of self-directed inquiry and experiential processing may be demonstrated and/or practiced.

In some embodiments, breathing exercises meant to promote calm and/or ease anxiety may be demonstrated and/or practiced. In some embodiments, the breathing exercise comprise instructing the subject to focus on their breath and/or sensations associated with the breath throughout the body. For example the subject may be instructed to breathe in for a count of four, to hold their breath for a moment, and then to breathe out for a count of eight. In some embodiments, the therapist and subject may discuss the most helpful ways to support in case of emotional distress during the psilocybin session. In some embodiments, the subject is given access (e.g., online access) to materials concerning the safety and mechanism of action of psilocybin.

In some embodiments, the pre-administration psychological support sessions will serve to establish a therapeutic goal for the psilocybin session. In some embodiments, the subject suggests the therapeutic goal for herself or himself. In some embodiments, the therapist suggests the therapeutic goal to the subject. In some embodiments, the subject is reminded of the therapeutic goal during the pre-administration psychological support session.

In some embodiments, the therapists are trained to counsel the subject before, during, and/or after the psilocybin sessions. In some embodiments, the therapist will have mental health training. In some embodiments, the therapist will be a clinical psychologist, a psychiatrist, a social worker, a doctor or a nurse. In some embodiments, the therapist will meet the following criteria:
Demonstrate independent clinical experience with direct subject care in areas that require counselling and psychotherapeutic skills;
Current unrestricted professional license and/or good professional standing with no history of suspension, professional misconduct or disciplinary actions; and/or.
High level of openness to learning new approaches and receiving feedback.

### Psychological Support During Psilocybin Sessions

During the treatment session, the subject may be supervised by one or more trained therapists. The therapist supervising the subject during the psilocybin session may be the same therapist from the subject's pre-administration psychological support session(s), or may be a different therapist. The therapist(s) may provide psychological support to the subject as necessary. As used herein, the term “psychological support” refers to any measure(s) taken by the therapist during the subject's psilocybin session to ensure the safety of the subject and maximize the clinical effectiveness of the psilocybin session. For example, the psychological support may be anything done by the therapist to (1) to ensure psychological safety of the subject; (2) to allow the subject's subjective experience to unfold naturally within the boundaries of the therapeutic intention set at the preparation; (3) to maintain participant's attention and awareness on the experience of the present moment thus allowing exposure and processing of the challenging emotional states and personal memories; and/or (4) to generate insights and solutions for the resolution of challenging personal situations, conflicts and traumatic experiences. In some embodiments, support can be in the form of therapeutic touch, verbal reassurance, guided imagery and/or relaxation or breathing exercises. In some embodiments, the support may comprise reminders, encouragement, or active guiding. Typically, only one technique is applied at a time to allow for minimal intervention and interference with the subject's unique process.

In some embodiments, the main therapeutic goals of the therapist during the psilocybin session are to (i) minimize extreme anxiety, and (ii) provide appropriate support that enables the skills and processes of self-directed inquiry and experiential processing. In some embodiments, the therapist demonstrates genuine presence, patience, curiosity, and/or openness during the psilocybin session. “Presence” refers to being totally available and present with the subject during all stages of the psilocybin session, and exuding calmness at all times. “Curiosity” refers to interest and willingness to understand the subject's experience, without making assumptions. “Patience” means that the therapist facilitates the participant taking as much time as needed to explore their experiences without controlling the natural urge to help or direct the experience. “Openness” is the ability of the therapist to remain cognitively and experientially open, including a capacity to be curious about how the subject's mind may uniquely choreograph the unfolding content of a session. This includes welcoming all emotions and expressions that might occur.

In some embodiments, the psychological support may comprise curious questioning. In this technique, brief, but detailed, questioning of subjects is used to help the subjects shift and sustain their attention towards different levels of cognition and emotions (“How does that make you feel?”) Due to the applicability across a range of mental states and within various settings, the technique of curious questioning can typically be used safely and consistently during the psilocybin session, regardless of the quality or intensity of the experience of each subject.

In some embodiments, the level of psychological support will vary during the various stages of the subject's psilocybin experience (e.g., the initial stage, the early stage, the peak stage, and the late stage). In some embodiments, the type of psychological support will vary during the various stages of the subject's psilocybin experience (e.g., the initial stage, the early stage, the peak stage, and the late stage). Because non-dual, ego-dissolution or “unitive” experiences have been shown to positively correlate with the magnitude and durability of the clinical response, the therapist will, in some embodiments, attend to such states with particular care.

In some embodiments, a subject may experience of a compromised sense of self during the subject's psilocybin experience. In some embodiments, this is interpreted from a psychoanalytic perspective as a disruption of ego-boundaries, which results in a blurring of the distinction between self-representation and object-representation, and precludes the synthesis of self-representations into a coherent whole. In some embodiments, non-dual, ego-dissolution or “unitive” experiences refer to an altered state of consciousness in which there is a reduction in the self-referential awareness that defines normal waking consciousness, resulting in a compromised sense of “self” and instead only a undivided background awareness, often characterised by a sense of unity or “oneness” that exceeds sensory or cognitive apprehension. In some embodiments, a non-dual experience is state of consciousness in which the subject-object dichotomy in normal waking consciousness is substituted for a unified background awareness that is centreless and undivided. In some embodiments, an ego dissolution experience is a spontaneously occurring state of consciousness where there is a reduction in the self-referential awareness that defines normal waking consciousness, resulting in a compromised sense of “self”. In some embodiments, a unitive experience is an experience characterised by a sense of unity or “oneness” that exceeds sensory or cognitive apprehension.

At the initial and early stage of the psilocybin session, psychological support may be used to reduce severe and/or prolonged anxiety. Anxiety prior to or during the onset of psilocybin effects is not uncommon, and the therapists may be specially trained to recognize and actively manage subjects through such periods of anxiety until the subject is comfortable enough to continue on their own. In some embodiments, therapists validate the subject's feelings of anxiety without providing interpretations of perceptual disturbances or guiding subjects towards a particular image or memory, other than encouraging them to stay relaxed and open to the emergent experiences. For example, in some embodiments, the therapist may help alleviate anxiety using a grounding exercise. In such an exercise, the subject may be encouraged to pay attention to the sounds around them or to sensations on their skin when touching the bed/couch, ground, or other objects.

At the initial and early stage of the psilocybin session, the therapist may encourage the subject to lie down, practice relaxation and breathing exercises, and/or listen to calming music. In some embodiments, the therapist may remind the subject of the intention for the treatment session. For example, the therapist may ask the subject “What does feeling better or recovery feel like?” or any number of similar questions. Such reminders prior to the onset of or at the onset of psilocybin effects provide an implicit direction for the subjective experience during the psilocybin session. In some embodiments, the therapist may remind the subject that their primary task during this session is to simply collect new and interesting experiences which can then be discussed with the therapist after the session. The therapist may remind the participant of the purpose of the psilocybin therapy and the role of experiential processing, namely allowing the participant to be open and curious to whatever arises and encountering thoughts and feelings previously unknown to them. In some embodiments, the therapist emphasizes that this process inherently requires letting go and a willing passivity to the psychedelic experience.

During the acute onset of action, the subject might experience perceptual changes in visual, auditory or olfactory modes, and a range of unusual physical sensations. These experiences could be anxiety-provoking. In some embodiments, the therapist may practice reassuring “arm holding”. This is where, upon the subject's request, a therapist will place his or her hand on the subject's wrist, arm, hand, or shoulder, as a way of helping the subject feel secure during this phase. This exercise may have been previously practiced during the pre-administration psychological support session.

In some embodiments, the therapist may encourage the subject to put on an eye mask, such as a Mindfold eyeshade. In some embodiments, the therapist encourages the subject to put on the eye mask before, during, or after the onset of the psilocybin's effects.

In some embodiments, the therapist may encourage the subject to put on headphones and listen to music. In some embodiments, the headphones reduce outside noise (e.g., “noise-cancelling” headphones). In some embodiments, the music is calming music such as instrumental (e.g., classical) music. In some embodiments, the music comprises nature sounds and/or the sound of moving water (e.g., ocean sounds). In some embodiments, the music comprises isochronic tones. In some embodiments, the music comprises moments of silence. In some embodiments, the music is emotionally evocative. In some embodiments, the music comprises a playlist which mirrors the pharmacodynamics of a typical high-dose psilocybin session: the initial stage, the early stage, the peak stage, and the late stage. In some embodiments, listening to music helps the subject to focus on their internal experience.

In case of prolonged anxiety or distress, therapists may, in some embodiments, actively guide participants through such experiences without interpreting or judging the experiences or giving advice. Once participants are comfortable, the therapist may encourage them to again engage in introspection.

During the peak and late stages of the psilocybin session, the therapist may encourage subjects to face and explore their experience, including the challenging ones. Therapists may direct subjects to participate self-directed inquiry and experiential processing to develop a different perspective on their personal challenges and conflicts, and to generate their own solutions. Such self-generated insights are not only therapeutic because of the emotional resolution, but also empowering to subjects.

As used herein, the term “self-directed inquiry” refers to directing attention to internal states. Subjects are encouraged to be curious about experiences in the present moment, including foreground and background thoughts, emotions, and physical sensations. During the preparation and integration stages, this inquiry might mean asking specific and detailed questions to help direct attention to internal states. However, during the period of drug action, inquiry might simply mean an attitude of openness to inner experiences.

As used herein, “experiential processing” refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the therapist will employ a transdiagnostic therapy. In some embodiments, the transdiagnostic therapy is a Method of Levels (MOL) therapy. In still further embodiments, the MOL therapy comprises Self-Directed Enquiry and Experiential Processing. Typically, MOL uses brief, but detailed, curious questioning to help subjects shift and sustain their attention towards different levels of cognition and emotions (Carey, 2006; Carey, Mansell & Tai, 2015). The emphasis within MOL is on identifying and working with a subject's underlying distress as opposed to just their symptoms. Such MOL related methods and techniques can include: (1) Self-directed enquiry-directing attention to internal states. Participants are encouraged to be curious about experiences in the present moment, including foreground and background thoughts, emotions, and physical sensations; during the preparation and integration stages, such enquiry can mean asking specific and detailed questions to help direct attention to internal states, although for some embodiments, during the period of drug action, enquiry can refer to an attitude of openness to inner experiences; and (2) Experiential processing-sustained focus on the experience; refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the psychological support comprises mindfulness-based therapy or CBT cognitive behavioral therapy (CBT). In some embodiments, the psychological support is informed by a functional theory of human behavior called Perceptual Control Theory.

Occasionally, the subject will try to avoid emerging experiences or distract him/herself while trying to regain cognitive control over the unusual state of their mind. Such distractions may take different forms. For example, the subject might want to engage in a conversation or prematurely describe in detail their experience, visions or insights. When this occurs, the therapist may aim to remain as silent as possible, thereby enabling the subject and his/her inner experience to direct the course of the psilocybin session. In some embodiments, the therapist may use active listening skills paired with prompts to encourage the subject to continue focusing attention on present experiences, particularly if the participant engages the therapist in conversation. In another example, a subject might ask to go to the bathroom or have a drink of water. The sudden and urgent character of such requests might suggest that they are really trying to avoid emerging material. In such cases, the therapist may encourage the subject to stay with the experience by simply redirecting their attention. For example, the therapist may say something like, “We will take a bathroom break at the end of this piece of music” or “I will get you water in a little while. Why don't you put the eye shades back on and relax for a few minutes?” If the subject is trying to avoid a difficult experience, they might listen to the suggestion and relax.

In some embodiments, spontaneous movement such as shaking, stretching or dancing while engaging with the experience is accepted and often encouraged, unless the movement seems to be a way to distract oneself from the experience. In some embodiments, if the subject continues to move around a lot, reminders to periodically return to a lying down position and to actively focus inwards may be provided.

The therapist is not required to understand, support or even have an opinion about the nature or content of the subject's experiences, but the therapist may validate them and convey openness toward the subject's own view of them without dismissing or pathologizing any experience based on its unusual content. These experiences may provide the subject with a perspective that goes beyond identification with their personal narrative. In some embodiments, the therapist will validate one or more of the subject's experiences. In some embodiments, validation of the experiences simply means acknowledging the courage of opening up to the experience and the possibility that any experience will serve the intention of the session.

In some embodiments, a therapist provides psychological support for approximately 4-8 hours immediately after administration of the psilocybin. In some embodiments, the therapist uses guided imagery and/or breathing exercises to calm the subject and/or focus the subject's attention. In some embodiments, the therapist holds the hand, arm, or shoulder of the subject. In some embodiments, the therapist counsels the subject to do one or more of the following: (1) to accept feelings of anxiety, (2) to allow the experience to unfold naturally, (3) to avoid psychologically resisting the experience, (4) to relax, and/or (5) to explore the subject's own mental space.

In some embodiments, the therapist avoids initiating conversation with the subject, but responds if the subject initiates conversation. Typically, active intervention is kept to a minimum during the treatment experience. In some embodiments, the subject is encouraged to explore their own mental space, and simple guided imagery may be used to assist relaxation. “Guided imagery” refers to an exercise wherein the subject is asked to imagine a scene (e.g., “Invite a scene, perhaps a landscape, and tell me where you find yourself”; “Imagine a place that feels safe to you.”)

### Post-Administration Psychological Support Session

In some embodiments, subjects may be encouraged to engage in post-administration integration sessions with their therapist. Integration is a process that involves processing, or embodying, a psychedelic experience within a therapeutic context. The process initially begins by the subject verbalising and reflecting upon any experience from the psilocybin session, and discussing it openly with their therapist. Successful integration of a psilocybin experience accommodates for emotional changes and comprises of translating experiences into new insights, perspectives, and subsequently new behaviors that can be used to benefit the subject's quality of life. New perspectives might in turn influence the participant's current knowledge or values and lead to new ways of relating to cognitions, emotions, behaviors and physical experiences.

In some embodiments, the goals and supportive methods used by the therapist throughout integration sessions should remain consistent, regardless of the intensity or content of the subjective experience explored by the subject. That said, the methods of support used by the therapist should accommodate for the full range of experiences a subject might have faced.

The integration process is not one that should be limited to the sessions with the therapist, and is a process that will likely continue to unfold beyond the visits in clinic. The therapist might encourage the participant to use methods such as spending time in nature, exercise, or creative expression to help facilitate the process further. The subject might also be encouraged to discuss experiences with their friends, family, and/or support network. The role of the integration sessions is not to cover and work on every experience, but to empower the participant by building their capacity to experientially process information safely. This enables the patient to continue self-directed integration, even outside of study visits.

In some embodiments, the subject participates in at least one psychological support session after administration of the psilocybin (“post-administration psychological support session”). In some embodiments, a post-administration psychological support session may be held on the same day as the psilocybin session, after the effects of the psilocybin have substantially worn off. In some embodiments, a post-administration psychological support session may be held the day after the psilocybin session. In some embodiments, a post-administration psychological support session may be held two days after the psilocybin session. In some embodiments, a post-administration psychological support session may be held three days after the psilocybin session. In some embodiments, a post-administration psychological support session may be held about one week after the psilocybin session. In some embodiments, a post-administration psychological support session may be held about two weeks after the psilocybin session. In some embodiments, a post-administration psychological support session may be held about one month after the psilocybin session. In some embodiments, a post-administration psychological support session may be held about three months after the psilocybin session. In some embodiments, a post-administration psychological support session may be held about six months after the psilocybin session. In some embodiments, a post-administration psychological support session may be held about twelve months after the psilocybin session.

In some embodiments, the subject may participate in one, two, three, four, five, six, seven, or eight post-administration psychological support sessions. In some embodiments, the subject may participate in at least two, or at least three post-administration psychological support sessions.

The post-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the post-administration psychological support session(s).

In some embodiments, the post-administration psychological support session may focus on integration of the psilocybin experience. Integration may involve processing a psychedelic experience in a therapeutic context. Integration may comprise psychological and somatic processing of the experience and a successful assimilation of insights into the subject's life for the purpose of growth, healing and/or well-being. During an integration session, a subject may be encouraged to talk about and reflect upon their experiences during the psilocybin session. In some embodiments, integration may comprise an external expression of the psilocybin experience, such as choice of words, tone of voice, gestures, and/or particular physical activities (yoga, exercise, bodywork, etc.) In some embodiments, integration comprises creatively expressing any insights or experiences gained during a psilocybin experience, for example through poetry, art, music/singing, dance, writing or drawing.

In some embodiments, the subject may be encouraged to reflect on both the thoughts and the feelings that he or she underwent during the psilocybin session, as well as to express those ideas and emotions into a concrete form that can serve as a tool for continuing to remember and integrate those lessons into the future. In some embodiments, the subject may be encouraged to acknowledge and connect with the range of the emotional cognitive and physical experiences of the psilocybin session, and relate them to current experiences in their life situation. This may be accomplished, for example, by discussing them initially with their therapist, and perhaps later with their family, friends, and support circle. Integration helps accommodate changes in emotional states as new insights are generated and integrated. When further explored through oscillating attention between foreground and background thoughts and emotions, such insights may lead to natural and effortless changes in perspectives or behaviors. In some embodiments, the integration process is not limited to initial integration meetings with the therapist, but continues to unfold spontaneously through a participant's own processing and actions in everyday life.

In the case of a low-intensity experience, the integration process might focus on the mental content that emerged during the hours of relaxation and introspection. This might also include reactions to what might have been an unremarkable experience, such as feeling of disappointment, anger, relief etc.

### Psychological Support Provided Remotely

In some embodiments, psychological support may be provided remotely to a subject. For example, a therapist providing psychological support may not be in the same room, the same building, or in the same facility as a subject. Remote psychological support may be provided, for example by telephone (i.e., by voice call), by video call or video conference, by text, or by email.

In some embodiments, a pre-administration therapy session is conducted remotely. In some embodiments, a post-administration therapy session (e.g., an integration session) is conducted remotely.

In some embodiments, psychological support is provided remotely during the subject's psilocybin session. For example, in some embodiments, the subject takes the psilocybin in his or her own home, and a therapist provides psychological support by voice call, video call, text, email, etc., for at least 4-8 hours after the subject has taken the drug. In some embodiments, the subject takes the psilocybin in an administration facility as described herein, and the therapist provides psychological support to the subject a therapist provides psychological support by voice call, video call, text, email, etc., for at least 4-8 hours after the subject has taken the drug

In some embodiments, remote psychological support is provided to the subject using a digital or electronic system. In some embodiments, the digital or electronic system may comprise one or more of the following features:
The digital or electronic system securely connects patients with one or more therapists or physicians for “virtual visits.” These virtual visits may be introductory or routine.
The digital or electronic system allows a subject to qualify, prequalify, or register for a psilocybin-based clinical trial, or a psilocybin-based psychological support session.
The digital or electronic system is configured to help therapists and/or physicians manage and interact with patients. For example, the electronic system may allow the therapist to share documents with subjects, keep notes about sessions, or schedule future sessions.
The digital or electronic system is configured to provide alerts for crisis intervention. For example, the digital or electronic system may allow the subject to contact the therapist if they are feeling anxiety or otherwise urgently need to talk to the therapist.
The digital or electronic system is configured to help prepare the subject for a visit with their therapist and/or physician. For example, the digital or electronic system may contain information regarding psilocybin, the therapeutic protocol, etc.
The digital or electronic system is configured to allow the therapist to provide psychological support during the subject's psilocybin session. For example, the system may comprise a video calling or chat feature.
The digital or electronic system is configured to allow the therapist to provide psychological support during a post-administration session (e.g., an integration session).
The digital or electronic system is configured to track the subject's adherence to the treatment regimen or goals.
The digital or electronic system is configured to assess one or more clinical endpoints in the subject. For example the system may comprise one or more questionnaires or exercises for the subject to complete. Results may be made available to the subject's physician and/or therapist.

In some embodiments, the digital or electronic system is an “app” for use on a mobile phone or a computer. In some embodiments, the digital or electronic system is a website. In some embodiments, the digital or electronic system comprises a “chat” feature which allows communication between the subject and the therapist in real time. In some embodiments, the website comprises a video calling feature, which allows for the therapist to communicate with the subject using video communication. In some embodiments, the digital or electronic system is configured to allow a single therapist to provide psychological support to one or more subjects at or around the same time.

In some embodiments, psychological support sessions may be pre-recorded (e.g., audio or video recording) and provided to the subject for use at the subject's convenience via the digital or electronic system.

### Administration Facility, “Set and Setting”

As used herein, the term “set and setting” refers to the subject's mindset (“set”) and the physical and social environment (“setting”) in which the user has the psilocybin session. In some embodiments, the psilocybin may be administered in a particular set and setting. In some embodiments, the set and setting is controlled, to the extent possible, to maximize therapeutic benefit of the psilocybin session.

In some embodiments, the psilocybin is administered by in a facility specifically designed for psilocybin administration. Administration of the psilocybin to the subject in a facility where the subject feels safe and comfortable may help ease anxiety in the subject, and may facilitate maximum clinical benefit. Psilocybin may be administered to a subject, for example, in the subject's home or at a clinical facility.

In some embodiments, the psilocybin is administered to the subject in a facility (e.g., a room) with a substantially non-clinical appearance. For example, the psilocybin can be administered in a room that comprises soft furniture (e.g., plush couches, chairs, or pillows) and/or plants. In some embodiments, the room may be decorated using muted colors (e.g., greyed, dulled, or desaturated colors). In some embodiments, the light in the room is dimmed and/or light levels are kept or adjust to be relatively low. In some embodiments, the room lighting is adjusted for intensity and/or color. In some embodiments, a virtual reality or augmented reality system (e.g., computer with visual/graphical and auditory outputs) is used. In some embodiments, the room comprises a sound system, for example a high-resolution sound system. In some embodiments, the sound system can allow for simultaneous ambient and earphone listening. In some embodiments, the subject may bring meaningful photographs or objects into the administration room.

In some embodiments, the room comprises a couch. In some embodiments, the room comprises a bed. In some embodiments the room comprises more than one couch or bed, such as 2, 3, 4, 5, 6, 7, 8, 9, or 10 couches or beds. In some embodiments, the subject sits on or lies in the couch or bed for approximately 4-8 hours, or a substantial fraction thereof, immediately after administration of the psilocybin. In some embodiments, the subject listens to music for approximately 4-8 hours, or a substantial fraction thereof, immediately after administration of the psilocybin. In some embodiments, the subject wears an eye mask for approximately 4-8 hours, or a substantial fraction thereof, immediately after administration of the psilocybin. In some embodiments, the subject is provided with a weighted blanket.

In some embodiments, each subject is supervised by one therapist during the psilocybin session. In some embodiments, each subject is supervised by more than one therapist during the psilocybin session, such as two therapists, three therapists, four therapists, or five therapists. In some embodiments, one therapist multiple subjects, wherein each subject is participating in a psilocybin session. For example, one therapist may supervise two, three, four, five, six, seven, eight, nine, or ten subjects.

Embodiments of the disclosure include use of additional tools and/or technique(s) with dosage/administration, including various transcranial magnetic stimulation (TMS) methods and protocols, for example, prior or subsequent to one or more dosing(s), biofeedback devices, etc.

Some embodiments can be used with a digital health product or digital solution. Teachings of the disclosure include utilization of such digital health products and/or related digital biomarkers as diagnostic and/or prognostic tools for patient monitoring and management pre-treatment, during treatment, and/or post treatment. Digital biomarkers can include, by way of non-limiting example: Number of and/or time of phone calls/e-mails/texts; word length in text communication; Gestures used (taps, swipes, or other); Gyroscope derived information e.g. orientation of the phone; Acceleration of the phone; Keystroke patterns; Location derived information from GPS; facial expressions and/or microexpressions; voice or vocal markers; natural language processing; social media use; sleep patterns; specific words or emojis used or not used; and/or the like. For example, in one embodiment, a digital health product can be utilized to determine dosing amount and/or dosing frequency, indicator of a need for re-dosing, re-dosing amount, a warning or alert, as tracking of compliance, etc.

In some embodiments, methods of treatment can include providing a clearance time for a subject or patient, such one or more medications is not present or substantially cleared from the system of the subject/patient. For example, methods of treatment can be configured such that, upon administration, the subject is not taking other serotonergic medications such as: selective-serotonin reuptake inhibitors, selective norepinephrine reuptake inhibitors, tricyclic antidepressants, monoamine oxidase inhibitors and/or antipsychotics. In some embodiment, the method of treatment include treatment concurrently with one or more medications, including but not limited to selective-serotonin reuptake inhibitors, selective norepinephrine reuptake inhibitors, tricyclic antidepressants, and/or monoamine oxidase inhibitors. In some embodiments, the method include treatment such that subjects or patients take concomitant compounds or medications, including but not limited to benzodiazepines, cannabidiol (CBD) and/or other cannabinoids (e.g., THC (tetrahydrocannabinol); THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); CBT (cannabicitran); and/or the like) magnesium, Levomefolic acid, e.g., for a period of time prior to, just prior to, and/or at the same time as receiving psilocybin.

In some embodiments, the method includes treatment such that a subject has not taken one or more medications, particularly has not taken one or more serotonergic medications for at least 2 days, at least, 3 days, at least 4 days, at least 5 days, at least six days, at least 1 week, at least 2, 3, or 4 weeks before administration of the disclosed psilocybin compound.

In some embodiments, the method and/or treatment can comprise subperceptual-dosing (e.g., a dose of less than 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, or 0.1 mg) prior to and/or following the administration of a relatively larger single dose or multiple doses (given a few days to a few weeks apart), where the relatively larger single dose or multiple doses is one or more of 5 mg or more, 10 mg or more, 15 mg or more, 20 mg or more, 25 mg or more, 30 mg or more, 35 mg or more, 40 mg or more, 45 mg or more, 50 mg or more.

Embodiments of the disclosure include method utilizing a digital biomarker, for example, as a diagnostic and/or prognostic tool for patient management pre-, during and/or post treatment with psilocybin wherein the digital biomarker is one or more biomarkers associated with executive function, cognitive control, working memory, processing speed, and/or emotional valence.

In some embodiments the digital biomarker is identified from patterns in smartphone use such as swipes, taps, and other touchscreen activities, and can be scientifically validated to provide measurements of subject status, such as cognition and mood, including, by way of non-limiting example, as disclosed in one or more of the following, each of which is herein expressly incorporated by reference for all purposes: US20170086727, US20170258382, US20170258383, US20170287348, U.S. Pat. Nos. 10,148,534, 9,737,759, and/or 10,231,651.

Biomarkers which may serve as a diagnostic and/or prognostic tool for patient management pre, during and/or post treatment may be identified using one or more of: Number of and/or time of phone calls/e-mails/texts; word length in text communication; Gestures used (taps, swipes, or other); Gyroscope derived information e.g. orientation of the phone; Acceleration of the phone; Keystroke patterns; Location derived information from GPS; facial expressions and/or microexpressions; voice or vocal markers; natural language processing; social media use; sleep patterns; specific words or emojis used or not used; and/or the like. In some embodiments, health components and/or connected biomonitors and/or smart devices/wearables can be utilized to collect information to be used in diagnostic and/or prognostic outputs. For example, in some embodiments, a heart rate monitor or similar device can collect a subject's data and heart rate variability (for example only, as disclosed in U.S. Pat. No. 10,058,253, the entirety of which is herein incorporated by reference) can be used to assess/determine a metric relating to the subject's current emotional state, relative change in emotional state, etc., which can be used in determining a new or follow-on treatment plan, adjusting a treatment plan, etc.

In accordance with a further aspect of the disclosure there is provided a method of assessing a subject pre, during and/or post treatment of a central nervous system disorder to determine whether to provide a psilocybin treatment or a further psilocybin treatment comprising monitoring one or more biomarkers associated with executive function, cognitive control, working memory, processing speed, and emotional valence, and determining the treatment based on an outcome. The method can further comprise the step of administering psilocybin for a first or a subsequent time.

In some embodiments, the biomarker is identified from patterns in smartphone use such as swipes, taps, and other touchscreen activities, and are scientifically validated to provide measurements of cognition and mood. For example, in some instances, the pattern is identified using one or more of: Number of and/or time of phone calls/e-mails/texts; word length in text communication; Gestures used (taps, swipes, or other); Gyroscope derived information e.g. orientation of the phone; Acceleration of the phone; Keystroke patterns; Location derived information from GPS; facial expressions and/or microexpressions; voice or vocal markers; natural language processing; social media use; sleep patterns; specific words or emojis used or not used; and/or the like.

Embodiments include a method of assessing a subject pre, during and/or post treatment of a central nervous system disorder to determine whether to provide a psilocybin treatment or a further psilocybin treatment comprising monitoring one or more biomarkers associated with executive function, cognitive control, working memory, processing speed, and emotional valence, and determining the treatment based on an outcome; the method can further comprise administering psilocybin for a first or a subsequent time.

In some embodiments, the disclosure provides for treating 2 or more subjects, the method comprising administering to each subject a therapeutically-effective dose of psilocybin at the same time or substantially the same time (e.g., dosed within several minutes of each other, within 5, 10, 15, 20, 25, or 30 min of each other), wherein each subject is aware of the other subject also receiving treatment. In some embodiments, the subjects are in the same room. In some embodiments, the subjects are in different rooms.

In some embodiments, the disclosure provides a method of treating a subject, the method comprising administering to the subject a therapeutically-effective dose of psilocybin, and providing a virtual reality/immersive reality digital tool. In some embodiments, the light in the room is dimmed and/or light levels are kept or adjusted to be relatively low. In some embodiments, darkened glasses or eye shades are provided. In some embodiments, the room lighting is adjusted for intensity and/or color. In some embodiments, a virtual reality or augmented reality system (e.g., computer with visual/graphical and auditory outputs) is used.

### Subjects

In some embodiments, the subject is a male. In some embodiments, the subject is a female. In some embodiments, the female subject is pregnant or post-partum. In some embodiments, the subject is attempting to reduce or eliminate their use of a pharmaceutical agent, such as an anti-depressant or an anti-epileptic drug. In some embodiments, the subject is attempting to reduce or eliminate their use of the pharmaceutical agent before becoming pregnant, having surgery or other medical procedure, or starting to use different pharmaceutical agent.

The subject may be a geriatric subject, a pediatric subject, a teenage subject, a young adult subject, or a middle aged subject. In some embodiments, the subject is less than about 18 years of age. In some embodiments, the subject is at least about 18 years of age. In some embodiments, the subject is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age.

The subject may have a chronic disease or a terminal disease. In some embodiments, the subject may have a life-altering disease or condition (such as the loss of a limb or onset of blindness).

The subject may have recently been diagnosed with a disease, disorder, or condition. For example, the subject may have been diagnosed within 1 month, within 3 months, within 6 months, or within 1 year. In some embodiments, the subject may have been living with a disease, disorder, or condition for an extended period time, such as at least 6 months, at least 1 year, at least 3 years, at least 5 years, or at least 10 years.

In some embodiments, the subject may be a cancer patient, such as a Stage 4 or terminal cancer patient. In some embodiments, the subject may have been determined to have a limited time to live, such as less than 1 year, less than 6 months, or less than 3 months.

The subject may have previously taken a psychedelic drug, or may have never previously taken a psychedelic drug. For example, the subject may or may not have previously taken psilocybin, a psilocybin mushroom (“magic mushroom”), LSD (lysergic acid diethylamide or acid), mescaline, or DMT (N,N-Dimethyltryptamine).

In some embodiments, the subject may have previously taken one or more serotonergic antidepressants (e.g., selective serotonin reuptake inhibitors (SSRIs)). In some embodiments, the subject has never previously taken a serotonergic antidepressant. In some embodiments, the subject has not taken any serotonergic antidepressants for at least 2 weeks, at least 4 weeks, or at least 6 weeks prior to receiving psilocybin.

In some embodiments, the subject may have previously received electroconvulsive therapy (ECT). In some embodiments, the subject has not received any ECT for at least 2 weeks, at least 4 weeks, or at least 6 weeks prior to receiving psilocybin.

The subject may have a medical condition that prevents the subject from receiving a particular medical therapy (such as an SSRI or ECT). In some embodiments, the subject may have previously had an adverse reaction to a particular medical therapy (such as an SSRI or ECT). In some embodiments, a prior medical therapy (such as an SSRI or ECT) was not effective in treating a disease, disorder, or condition in the subject.

Diseases, Disorders, and/or Conditions to be Treated

Provided herein are methods of treating a subject in need thereof, the method comprising administering to the subject a therapeutically-effective dose of psilocybin.

The methods described herein may be used to treat a variety of diseases, disorders, or conditions including particular psychiatric and neurological aspects of the diseases, disorders, or conditions.

In some embodiments, the disclosure provides a method of treating a subject in need thereof, the method comprising administering to the subject a therapeutically-effective dose of psilocybin or a metabolite thereof, wherein the subject has at least one or more of the following diseases, disorders, or conditions: Disruptive Mood Dysregulation Disorder, Major Depressive Disorder (MDD), Treatment Resistant Depression, Persistent Depressive Disorder (Dysthymia), Premenstrual Dysphoric Disorder, Substance/Medication-Induced Depressive Disorder, Post-Partum Depression, Depressive Disorder due to Another Medical Condition, Separation Anxiety Disorder, Selective Mutism, Specific Phobia, Social Anxiety Disorder (Social Phobia), Panic Disorder, Panic Attack, Agoraphobia, Generalized Anxiety Disorder, Substance-Medication-Induced Anxiety Disorder, Anxiety Disorder Due to Another Medical Condition, Somatic Symptom Disorder, Illness Anxiety Disorder (hypochondriac), Conversion Disorder (Functional Neurological Symptom Disorder), Factitious Disorder, Post-Traumatic Stress Disorder (PTSD), Adjustment Disorders, Acute Distress Disorder, Obsessive-Compulsive Disorder, Body Dysmorphic Disorder, Hoarding Disorder, Trichotillomania (Hair-Pulling) Disorder, Excoriation (Skin-Picking) Disorder, Substance/Medication-Induced Obsessive-Compulsive and Related Disorder, Obsessive-Compulsive and Related Disorder due to Another Medical Condition, Substance-Related Disorders, Alcohol-Related Disorders, Cannabis-Related Disorders, Hallucinogen-Related Disorders, Inhalant-Related Disorders, Cocaine-Related Disorders, Opioid-Related Disorders, Sedative-, Hypnotic-, or Anxiolytic-Related Disorders, Stimulant-Related Disorders, Tobacco-Related Disorders, Non-Substance-Related Disorders (Gambling or Gaming Disorder), Migraines, Cluster Headaches such as Chronic Cluster Headaches, Cyclical Vomiting, Tension-Type Headache, Dysphasia, Pica, Anorexia Nervosa, Bulimia Nervosa, Binge-Eating Disorder, Oppositional Defiant Disorder, Intermittent Explosive Disorder, Conduct Disorder, Antisocial Personality Disorder, Psychopathy, Pyromania, or Kleptomania.

In some embodiments, the disclosure provides a method of treating a subject in need thereof, the method comprising administering to the subject a therapeutically-effective dose of psilocybin, wherein the subject has at least one of the following diseases, disorders, or conditions: a Neurocognitive Disorder due to Alzheimer's, Lewy Bodies, Traumatic Brain Injury, Prion Disease, Human Immunodeficiency Virus (HIV) Infection, Parkinson's, Huntington's; concussion; Chronic Traumatic Encephalopathy (CTE); Language Disorder, Speech Sound Disorder (Phonological Disorder); Childhood-Onset Fluency Disorder (Stuttering); Social (Pragmatic) Communication Disorder; Tourette's Disorder; Persistent (Chronic) Motor or Vocal Tic Disorder; Amnestic Disorder Due to Known Physiological Condition (possibly in ECT shock-resistant subjects); Transient Cerebral Ischemic Attack, Cerebral Infarction, Cerebral Bleeding, Progressive Supranuclear Ophthalmoplegia, or Retrograde Amnesia.

In some embodiments, the disclosure provides a method of treating a subject in need thereof, the method comprising administering to the subject a therapeutically-effective dose of psilocybin, wherein the subject has at least one of the following diseases, disorders, or conditions: Autism, Autism Spectrum-Disorder, or Antisocial Personality Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Attention-Deficit/Hyperactivity Disorder, Other Specified Attention-Deficit/Hyperactivity Disorder or Unspecified Attention-Deficit/Hyperactivity Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Schizotypal (Personality) Disorder, Delusional Disorder, Schizophrenia, or Schizoaffective Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Insomnia Disorder, Hypersomnolence Disorder, Narcolepsy, or Primary Central Sleep Apnea.

In some embodiments, the disclosure provides a method of treating a subject in need thereof, the method comprising administering to the subject a therapeutically-effective dose of psilocybin, wherein the subject has at least one of the following diseases, disorders, or conditions: Schizoid Personality Disorder, Schizotypal Personality Disorder, Antisocial Personality Disorder, Borderline Personality Disorder, or Obsessive-Compulsive Personality Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Female Sexual Interest/Arousal Disorder, Male Hypoactive Sexual Desire Disorder, and Excessive Sexual Drive.

In some embodiments, the disclosure provides a method of treating a subject in need thereof, the method comprising administering to the subject a therapeutically-effective dose of psilocybin, wherein the subject has at least one of the following diseases, disorders, or conditions: Bipolar I Disorder, Bipolar II Disorder, or Cyclothymic Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Age-Related Hearing Loss or Tinnitus.

In some embodiments, the disclosure provides a method of treating a subject in need thereof, the method comprising administering to the subject a therapeutically-effective dose of psilocybin, wherein the has at least one of the following diseases, disorders, or conditions: Multiple Sclerosis, Cranial Nerve Disorder, Neuromyelitis Optica, Bell's Palsy, Guillain Barre Syndrome, Demyelinating Disease of Central Nervous System, or Chronic Inflammatory Demyelinating Polyneuritis.

In some embodiments, the disclosure provides a method of treating a subject in need thereof, the method comprising administering to the subject a therapeutically-effective dose of psilocybin, wherein the subject suffers from pain, such as phantom pain, chronic pain, or pain associated with another disease or disorder.

In some embodiments, the disclosure provides a method of treating a subject in need thereof, the method comprising administering to the subject a therapeutically-effective dose of psilocybin, wherein the subject has at least one of the following diseases, disorders, or conditions: Myelopathy, Traumatic Brain Injury, Intellectual Disabilities, Mania, Neurodegeneration, Paraphilic disorders (e.g., Pedophilic Disorder), Suicidal Behavior Disorder, Nonsuicidal Self-Injury, Persistent Complex Bereavement Disorder, GI Tract Related Diseases (e.g., IBS), Epilepsy, Sickle Cell Disease, locked-in syndrome, restless leg syndrome, stroke (such as ischemic stroke or hemorrhagic stroke), or Amyotrophic Lateral Sclerosis (ALS).

In some embodiments, the disclosure provides a method of treating a subject, the method comprising administering to the subject a therapeutically-effective dose of psilocybin, wherein after administration the subject exhibits an improvement in cognition. In some embodiments, the improvement in cognition is an improvement in attention, episodic memory, working memory, spatial memory, social cognition, executive function, and/or cognitive flexibility.

In some embodiments, the disclosure provides a method of treating a subject in need thereof, the method comprising administering to the subject a therapeutically-effective dose of psilocybin, wherein the subject has Treatment Resistant Depression (TRD).

In some embodiments, the disclosure provides a method of treating a subject in need thereof, the method comprising administering to the subject a therapeutically-effective dose of psilocybin, wherein the subject has Major Depressive Disorder (MDD).

### Pre-Treatments and Combination Therapies

In some embodiments, the methods of treatment comprising administering psilocybin to a subject in need thereof further comprise pretreating the subject with magnesium before administration of the psilocybin. Sometimes, magnesium is administered daily for a least 1 day, at least 2 days, at least 3 days, at least 4 days, at least 5 days, at least 6 days, at least 1 week, at least 2 weeks, at least 3 weeks, at least 4 weeks, at least 5 weeks, or at least 6 weeks before administration of the psilocybin. In some embodiments, about 10 mg to about 500 mg of magnesium are administered to the subject per day. In some embodiments, about 30 mg, about 75 mg, about 80 mg, about 130 mg, about 240 mg, about 310 mg, about 320 mg, about 360 mg, about 410 mg, about 400 mg, or about 420 mg are administered to the subject per day. In some embodiments the magnesium is administered to the subject on the same day as the psilocybin. In some embodiments, the magnesium is administered to the subject immediately before, concurrently with, or immediately after administration of the psilocybin. In some embodiments, magnesium supplements are administered to the subject until the subject's blood level for magnesium is about 1.5 to about 2.5 mEq/L. In some embodiments, psilocybin is not administered to the subject if the subject's blood level of magnesium is less than about 1.5 to about 2.5 mEq/L.

In some embodiments, the methods of treatment comprising administering psilocybin to a subject in need thereof further comprise pretreating the subject with niacin before administration of the psilocybin. Sometimes, niacin is administered daily for a least 1 day, at least 2 days, at least 3 days, at least 4 days, at least 5 days, at least 6 days, at least 1 week, at least 2 weeks, at least 3 weeks, at least 4 weeks, at least 5 weeks, or at least 6 weeks before administration of the psilocybin. In some embodiments, about 1 mg to about 5,000 mg of niacin are administered to the subject per day, for example about 1 mg to about 50 mg, about 10 mg to about 100 mg, about 100 mg to about 200 mg, about 1 mg to about 200 mg, about 100 mg to about 200 mg, about 10 mg to about 50 mg, about 10 to about 35 mg, about 100 mg to about 500 mg, or about 1,000 mg to about 3,000 mg. In some embodiments, about 10 mg, about 14 mg, about 15 mg, about 16 mg, about 20 mg, about 30 mg, about 35 mg, about 50 mg, about 60 mg, about 75 mg, about 100 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg of niacin are administered to the subject per day (while avoiding any toxic exposure from excess niacin). In some embodiments, niacin is included as an ingredient/component, for example, to reduce risk of abuse and/or to improve efficacy. In some embodiments the niacin is administered to the subject on the same day as the psilocybin. In some embodiments, the niacin is administered to the subject immediately before, concurrently with, or immediately after administration of the psilocybin.

In some embodiments, psilocybin is administered to the subject in combination with one or more additional therapies. In some embodiments, psilocybin is administered to the subject in combination with one or more anti-depressant or anti-anxiety drugs, such as SSRIs, tricyclic antidepressants (TCAs), monoamine oxidase inhibitors (MAOIs), or serotonin norepinephrine reuptake inhibitors (SNRIs).

In some embodiments, the disclosure provides a method of reducing anxiety in a subject undergoing treatment with psilocybin, the method comprising administering to the subject: i) psilocybin or a precursor or derivative thereof, and ii) one or more benzodiazepines.

In some embodiments, the one or more benzodiazepines are administered to the subject at or around the same time as the psilocybin or precursor or derivative thereof. In some embodiments, the one or more benzodiazepines are administered to the subject prior to administration of the psilocybin or precursor or derivative thereof, such as about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes before administration of the psilocybin or precursor or derivative thereof. In some embodiments, the one or more benzodiazepines are administered to the subject after the psilocybin or precursor or derivative thereof, such as about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes after administration of the psilocybin or precursor or derivative thereof.

In some embodiments, the one or more benzodiazepines are administered at a dose that is lower than doses typically used to treat anxiety, such as about 10%, 20%, 25%, 30%, 40%, 50%, or 75% of a typical dose. In some embodiments, the one or more benzodiazepines are administered at a dose that is approximately equivalent to doses typically used to treat anxiety. In some embodiments, the one or more benzodiazepines are administered at a dose that is higher than doses typically used to treat anxiety, such as about 125%, 150%, 175%, 200%, 250%, or 300% of a typical dose. In some embodiments, the one or more benzodiazepine is administered orally to the subject.

In some embodiments, the benzodiazepine is selected from the group consisting of adinazolam, alprazolam, bentazepam, bretazenil, bromazepam, bromazolam, brotizolam, camazepam, chlordiazepoxide, cinazepam, cinolazepam, clobazam, clonazepam, clonazolam, clorazepate, clotiazepam, cloxazolam, delorazepam, deschloroetizolam, diazepam, diclazepam, estazolam, ethyl carfluzepate, ethyl loflazepate, etizolam, flualprazolam, flubromazepam, flubromazolam, fluclotizolam, flunitrazepam, flunitrazolam, flurazepam, flutazolam, flutoprazepam, halazepam, ketazolam, loprazolam, lorazepam, lormetazepam, meclonazepam, medazepam, metizolam, mexazolam, midazolam, nifoxipam, nimetazepam, nitemazepam, nitrazepam, nitrazolam, nordiazepam, norflurazepam, oxazepam, phenazepam, pinazepam, prazepam, premazepam, pyrazolam, quazepam, rilmazafone, temazepam, tetrazepam, and triazolam.

In certain embodiments, a patient is administered psilocybin or a precursor or derivative thereof as described herein along with one or more 5-HT2A specific antagonists and/or inverse agonists. In some embodiments, the patient is administered psilocybin or a precursor or derivative thereof and the one or more 5-HT2A specific antagonists and/or inverse agonists at the same time. In other embodiments, the patient is administered one or more 5-HT2A specific antagonists and/or inverse agonists prior to psilocybin administration, such as, but not limited to about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes before psilocybin administration. In some embodiments, the patient is administered one or more 5-HT2A specific antagonists and/or inverse agonists after psilocybin administration, such as, but not limited to about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes after psilocybin administration.

In certain embodiments, the one or more 5-HT2A specific antagonists and/or inverse agonists are administered at doses that are lower than doses typically used, e.g., about 10%, about 20%, about 25%, about 30%, about 40%, about 50%, or about 75% of a typical dose. In other embodiments, the one or more 5-HT2A specific antagonists and/or inverse agonists are administered at doses that are equivalent to doses typically used. In yet other embodiments, the one or more 5-HT2A specific antagonists and/or inverse agonists are administered at doses that are higher than doses typically used, e.g., about 125%, about 150%, about 175%, about 200%, about 250%, or about 300% of a typical dose.

Suitable 5-HT2A antagonists include but are not limited to, trazodone, mirtazapine, metergoline, ketanserin, ritanserin, nefazodone, clozapine, olanzapine, quetiapine, risperidone, asenapine, MDL-100907, cyproheptadine, pizotifen, LY-367,265, 2-alkyl-4-aryl-tetrahydro-pyrimido-azepine, 9-aminomethyl-9,10-dihydroanthracene (AMDA), haloperidol, chlorpromazine, hydroxyzine (atarax), 5-MeO-NBpBrT, niaprazine, altanserin, aripiprazole, etoperidone, setoperone, chlorprothixene, cinaserin, adatanserin, medifoxamine, rauwolscine, phenoxybenzamine, pruvanserin, deramciclane, nelotanserin, lubazodone, mepiprazole, xylamidine, R-(+)-alpha-(2,3-dimethoxyphenyl)-1-[2-(4-fluorophenethyl)]-4-piperidinemethanol (M100907), mianserin, AT 1015, DV 7028, eplivanserin, 4F 4PP, fanaserin, alpha-phenyl-1-(2-phenylethyl)-4-piperidinemethanol (MDL 11,939), melperone, mesulergine, paliperidone, 1-[2-(3,4-Dihydro-1H-2-benzopyran-1-yl)ethyl]-4-(4-fluorophenyl) piperazine dihydrochloride (PNU 96415E), (2R,4R)-5-[2-[2-[2-(3-methoxyphenyl)ethyl]phenoxy]ethyl]-1-methyl-3-pyrrolidinol (R-96544), sarpogrelate, spiperone, ziprasidone, zotepine, and 7-[[4-[2-(4-fluorophenyl)ethyl]-1-piperazinyl]carbonyl]-1H-indole-3-carbonitrile (EMD 281014).

Suitable 5-HT_{2A} reverse agonists include but are not limited to, AC-90179, nelotanserin (APD-125), eplivanserin, pimavanserin (ACP-103), and volinaserin.

In certain embodiments, the 5-HT_{2A} antagonist is selected from the compounds of Table F.

TABLE F
5-HT2A antagonists

Acepromazine
Agomelatine
Amitriptyline
Amoxapine
Amperozide
APD791
Aripiprazole
Aripiprazole lauroxil
Blonanserin
Brexpiprazole
Butriptyline
Captodiame
Cariprazine
Chlorpromazine
Chlorprothixene
Cinitapride
Citalopram
Clomipramine
Clozapine
Cyclobenzaprine
Cyproheptadine
Deramciclane
Desipramine
Dosulepin
Doxepin
Epinastine
Esmirtazapine
Etoperidone
Flibanserin
Fluoxetine
Flupentixol
Fluspirilene
Iloperidone
Imipramine
Lisuride
Loxapine
Lurasidone
Mesoridazine
Methotrimeprazine
Methysergide
Mianserin
Mirtazapine
Nefazodone
Nortriptyline
Olanzapine
Paliperidone
Pimavanserin
Pizotifen
Promazine
Propiomazine
PRX-08066
Quetiapine
Risperidone
Sertindole
Thioproperazine
Thioridazine
Tramadol
Trazodone
Triflupromazine
Trimipramine
YKP-1358
Yohimbine
Ziprasidone
Zotepine
Zuclopenthixol

In some embodiments, the disclosure provides a method of reducing the negative side effects associated with a traumatic psychedelic experience in a patient undergoing treatment with psilocybin, the method comprising administering to the patient: i) psilocybin or a precursor or derivative thereof, and ii) one or more cannabinoids or cannabinoid derivatives.

In some embodiments, the cannabinoid is selected from the group consisting of THC (tetrahydrocannabinol), THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); and CBT (cannabicitran). In particular embodiments, the cannabinoid is CBD (cannabidiol).

In some embodiments, at least one symptom of a disease, disorder, or condition described herein is alleviated within 24 hours of administering psilocybin. In some embodiments, at least one symptom of the disease, disorder, or condition is alleviated within 1 week of the administering. In some embodiments, at least one symptom of the disease, disorder, or condition is alleviated within 1 month of the administering. In some embodiments, at least one symptom of the disease, disorder, or condition is alleviated within 6 months of the administering. In some embodiments, at least one symptom of the disease, disorder, or condition is alleviated within 12 months of the administering.

In some embodiments, at least one symptom of the disease, disorder, or condition is alleviated for a period of at least 1 month after administering psilocybin. In some embodiments, at least one symptom of the disease, disorder, or condition is alleviated for a period of at least 3 months after the administering. In some embodiments, at least one symptom of the disease, disorder, or condition is alleviated for a period of at least 6 months after the administering. In some embodiments, at least one symptom of the disease, disorder, or condition is alleviated for a period of at least 12 months after the administering.

In some embodiments, no other treatment is administered to the subject to treat the disease, disorder, or condition before administration of the psilocybin. In some embodiments, no other treatment is administered to the subject to treat the disease, disorder, or condition after administration of the psilocybin.

### Safety and Efficacy of Psilocybin

The present disclosure also relates to the safety and efficacy of the use of psilocybin as disclosed herein. The following is a non-exhaustive list of tests that can be used to determine the effects of psilocybin, and in particular the psilocybin formulations as disclosed herein administered as disclosed herein.

In some embodiments, the Spatial Working Memory (SWM) test is utilized to evaluate the safety and efficacy of psilocybin as disclosed herein. SWM requires retention and manipulation of visuospatial information. Study subjects are required to find the blue tokens in the on-screen ‘boxes’. Boxes are searched by touching them to determine whether they contain a token. Once a token has been located it is ‘stacked’ in a column on the right of the screen. Study subjects then search for further tokens until they have all been located. The remaining tokens will thereafter only be found in boxes that have not so far yielded a token. Study subjects are explicitly told this is the case and it they revisit a box in which a token has been found they commit a ‘between error’, the usual primary metric for this test. Occasions on which the subject revisits a box in the same search are scored as a ‘within’ error. Many study subjects will adopt a search strategy via which they systematically search the array of boxes. This is also scored by the Cambridge Neuropsychological Test Automated Battery system and yields a ‘strategy’ score. SWM performance is impaired by damage to the prefrontal cortex, especially the dorsolateral prefrontal cortex. Similarly, in neuroimaging studies in healthy volunteers, SWM performance is associated with activations in the dorsolateral and mid-ventrolateral prefrontal cortex. This test takes approximately 4 min to complete.

In some embodiments, the efficacy of psilocybin is evaluated using the spatial working memory between errors (SWMBE) score. In some embodiments, after treating according to the methods of the disclosure, a subject's SWMBE score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the efficacy of psilocybin is evaluated using the spatial working memory strategy (SWMS) score. In some embodiments, after treating according to the methods of the disclosure, a subject's SWMS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the Rapid Visual Information Processing (RVP) test is utilized to evaluate the safety and efficacy of psilocybin. The RVP is a measure of sustained attention outputting measures of response accuracy, target sensitivity and reaction times. In this test, the study subject is required to monitor a stream of digits from 2 to 9 for specific sequences (e.g., 3-5-7) and to acknowledge detection of the sequence by touching the on-screen response button as quickly as possible after presentation of the third digit. Digits are presented pseudorandomly to create the possibility of ‘false alarm’ responses in which the first 2 digits of a sequence are not followed by a true target, e.g., when 3 is followed by a 5, but not then by a 7. In order to complete the task successfully study subjects must sustain attention to the white box in which the digits appear. Performance on this task is measured by the speed of response to the presentation of the final digit of a target, as well as the study subject's ability to detect specified sequences. This test takes approximately 7 min to complete. In some embodiments, performance on the Rapid Visual Information Processing test is reported using a RVP A Prime (RPVA) score. Higher scores on the RVPA indicated better performance. In some embodiments, after treating according to the methods of the disclosure, a subject's RVPA score increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Paired Associates Learning (PAL) test is utilized to evaluate safety and/or efficacy of psilocybin. The PAL task is a measure of visuo-spatial memory in which study subjects are required to remember locations at which visual stimuli are located. Boxes are displayed on the screen and are “opened” in a randomized order. One or more of them will contain a pattern. The patterns are then displayed in the middle of the screen, one at a time and the subject must select the box in which the pattern was originally located. If the subject makes an error, the boxes are opened in sequence again to remind the subject of the locations of the patterns. Increased difficulty levels can be used to test high-functioning, healthy individuals. The primary metric for this test is the number of errors made. This test takes approximately 8 min to complete. Successful performance of the PAL test is dependent on functional integrity of the temporal lobe, particularly the entorhinal cortex. In some embodiments, the Paired Associates Learning total errors adjusted (PALTEA) score is used to assess the efficacy of psilocybin. In some embodiments, after treating according to the methods of the disclosure, a subject's PALTEA score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the efficacy and/or safety of psilocybin is evaluated using the cognitive flexibility panel test.

In some embodiments, the Emotion Recognition Task (ERT) test is utilized to evaluate the safety and/or efficacy of psilocybin. The ERT measures the ability to identify 6 basic emotions in facial expressions along a continuum of expression magnitude. In some embodiments, the ERT is performed according to the following protocol: Subjects are shown computer morphed images derived from the facial features of real individuals each showing a specific emotion, on a screen, one at a time. Each face is displayed for 200 ms and then immediately covered up, and the subject must select which emotion the face displayed from the six options (happy, sad, anger, fear, surprise, disgust). The ERT percent correct (ERTPC) of correct responses (emotion selection) the subject made is assessed. A higher score indicates better performance. In some embodiments, after treating according to the methods of the disclosure, a subject's ERTPC increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Intra-Extra Dimensional Set Shift (IED) test is used to evaluate the safety and/or efficacy of psilocybin. The IED consists of four 7-item subscales, each of which taps a separate aspect of the global concept “empathy.” In some embodiments, the Intra-Extra Dimensional Set Shift total errors (IEDYERT) score is used to assess the efficacy of psilocybin. In some embodiments, after treating according to the methods of the disclosure, a subject's IEDYERT score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the One Touch Stockings (OTS) of Cambridge test is used to evaluate the safety and/or efficacy of psilocybin. The OTS is a test of executive function, based upon the Tower of Hanoi test. It assesses both the spatial planning and the working memory subdomains. This test takes approximately 10 min to perform. The OTS test reports an one touch stockings of Cambridge problems solved on first choice (OTSPSFC) score. A higher OTSPSFC score is associated with better executive function. In some embodiments, after treatment according to the methods of the disclosure, a subject's OTSPSFC score increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, verbal fluency is used to evaluate the safety and/or efficacy of psilocybin. In the verbal fluency test, the study subject is asked to name as many different category exemplars (e.g., ‘animals’) as they can in 1 min, subject to certain scoring rules, such as repetition. Successful performance on this test is reliant on the integrity of a number of cognitive abilities and especially those traditionally viewed as executive functions, such as planning and working memory. The primary metric for this test is the total number of acceptable words generated. In some embodiments, after treatment with psilocybin, a subject's verbal fluency category score improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, the Digit Span Forward (DSF) test is used to evaluate the safety and/or efficacy of psilocybin. DSF is used to measure number storage capacity. Subjects hear a sequence of digits and are tasked to recall the sequence correctly, with increasingly longer sequences being tested in each trial. The subject's span is the longest number of sequential digits that can accurately be remembered. Digit span tasks can be given forwards or backwards, meaning that once the sequence is presented, the subject is asked to either recall the sequence in normal or reverse order. For this study, subjects will be asked to recall the sequence in the order presented, i.e., Digit Span Forward. The primary metric for this test is the number of digit sequences successfully recalled. In some embodiments, after treatment with psilocybin, a subject's Digit Span Forward score improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, the Five Dimension Altered States of Consciousness questionnaire (5D-ASC) is utilized to evaluate the safety and/or efficacy of psilocybin. The 5D-ASC measures the acute drug effects using 5 primary dimensions and 11 lower-order scales to assess alterations in mood, perception and experience of self in relation to environment and thought disorder. The 5 dimensions include oceanic boundlessness, anxious ego dissolution, visionary restructuralization, auditory alterations and reduction of vigilance. In some embodiments, after treatment according to the methods of the disclosure, a subject experiences an increase on a dimension or a subscale compared to prior to treatment. The lower-order scales include “experience of unity,” “spiritual experience,” “blissful state,” “insightfulness,” “disembodiment,” “impaired control of cognition,” “anxiety,” “complex imagery,” “elementary imagery,” “audio-visual synesthesia,” and “changed meaning of percepts.” In some embodiments, the increase is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Positive and Negative Affect Schedule (PANAS) is used to evaluate the safety and/or efficacy of psilocybin. The PANAS measures the acute emotional drug effects and comprises 2 mood scales that measure positive and negative affect. Positive affect refers to the propensity to experience positive emotions and interact with others positively. Negative affect involves experiencing the world in a more negative way. Subjects respond to 10 questions associated with negative affect and 10 questions associated with positive affect. The questions are scaled using a 5-point scale that ranges from “slightly or not at all (1)” to “extremely (5)”. A total higher score on the positive affect questions indicates more of a positive effect while a lower score on the negative affect questions indicates less of a negative affect. In some embodiments, after treating according to the methods of the disclosure, a subject experiences a decrease in negative affect score of the PANAS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment. In some embodiments, after treating according to the methods of the disclosure, a subject experiences an increase in positive affect score of the PANAS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Generalised Anxiety Disorder-7 item scale (GAD-7) is used to evaluate the safety and/or efficacy of psilocybin. The GAD-7 is useful in primary care and mental health settings as a screening tool and symptom severity measure for the seven most common anxiety disorders. Participants choose one of 4 severity scores associated problems related to the common anxiety disorders and then indicate the degree to which these problems caused functional and/or social difficulties. Scores are determined by calculating the values for each column. A total score is obtained by the sum of all total column values. In some embodiments, after treating according to the methods of the disclosure, a subject experiences a decrease in score of the GAD-7, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the 16-Item Quick Inventory of Depressive Symptomatology-Self Report (QIDS-SR-16) is used to evaluate the safety and/or efficacy of psilocybin. The 16-item QIDS-SR-16 a self-rated scale designed to assess the severity of depressive symptoms in the nine diagnostic symptom domains of a major depressive episodes, exclusive of atypical or melancholic symptoms. The QIDS-SR-16 is sensitive to change with various treatments, demonstrating its utility in research settings. The total score ranges from 0 to 27 with 0 representing no depression and 27 representing severe depression. The total score is the sum of the 9 symptom domains. In some embodiments, after treating according to the methods of the disclosure, a subject experiences a decrease in score of the QIDS-SR-16, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the Sheehan Disability Scale (SDS) is used to evaluate the safety and/or efficacy of psilocybin. The SDS is a brief, 5-item self-report inventory that assesses functional impairment in work/school, social life, and family life. The total score ranges from 0 to 30 with 0 representing no impairment and 30 representing severe impairment. In some embodiments, after treating according to the methods of the disclosure, a subject experiences a decrease in score of the SDS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the Work and Social Adjustment Scale (WSAS) is used to evaluate the safety and/or efficacy of psilocybin. The WSAS is a 5-item self-report scale used to assess psychosocial functioning and to predict durability of response to antidepressant treatment. Each of the 5 questions is rated on a scale from 0 to 8, where 0 is no impairment and 8 is very severe impairment. In some embodiments, after treating according to the methods of the disclosure, a subject experiences a decrease in score of the WSAS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the EuroQol-5-Dimension-3-Level Scale (EQ-5D-3L) is used to evaluate the safety and/or efficacy of psilocybin. The EQ-5D-3L system comprises five dimensions: mobility, self-care, usual activities, pain/discomfort and anxiety/depression. Each dimension has 3 levels: no problems, slight problems, moderate problems, severe problems and extreme problems. The participant is asked to indicate his/her health state by ticking the box next to the most appropriate statement in each of the five dimensions. This decision results in a 1-digit number that expresses the level selected for that dimension. The digits for the five dimensions can be combined into a 5-digit number that describes the participant's health state. In some embodiments, after treating according to the methods of the disclosure, a subject experiences an increase in score of the EQ-5D-3L, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the Digit Symbol Substitution Test (DSST) is used to evaluate the safety and/or efficacy of psilocybin. In some embodiments, after treating according to the methods of the disclosure, a subject experiences an increase in score of the DSST, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment.

In some embodiments, the NEO-Five Factor Inventory (NEO-FFI) test is used to evaluate the safety and/or efficacy of psilocybin. The NEO-FFI evaluates 5 broad domains of personality-Neuroticism, Extroversion, Openness, Agreeableness and Conscientiousness.

In some embodiments, the Symptom Checklist-90 item (SCL-90) questionnaire is used to evaluate the safety and/or efficacy of psilocybin. The SCL-90 is a relatively brief self-report psychometric instrument designed to evaluate a broad range of psychological problems and symptoms of psychopathology. In some embodiments, the SCL-90 is used to assess somatization, obsessive-compulsive behaviors, interpersonal sensitivity, depression, anxiety, hostility, phobic anxiety, paranoid ideation, and psychoticism of a subject treated according to the methods of the disclosure. The 90 items in the questionnaire are scored on a 5-point Likert scale, indicating the rate of occurrence of the symptom during the time reference. In some embodiments, after treating according to the methods of the disclosure, a subject's SCL-90 score decreases by about 5% to about 100%, for example, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Life Changes Inventory (LCI) questionnaire is utilized to evaluate the safety and/or efficacy of psilocybin. The LCI is designed as a questionnaire to investigate those variables present in the day-to-day experience of adults that might relate either to stability or decline of intellectual ability.

In some embodiments, Social Cognition Panel scales are utilized to evaluate the safety and/or efficacy of psilocybin. The social cognition panel scales comprise the pictorial empathy test (PET), reading the mind in the eyes test (RMET), social value orientation (SVO) test, the Toronto Empathy Questionnaire (TEQ), and the scale of social responsibility (SSR).

In some embodiments, the Pictorial Empathy Test (PET) is utilized to evaluate the effect of psilocybin on affective empathy.

In some embodiments, Reading the Mind in the Eyes Test (RMET) is utilized to evaluate the safety and/or efficacy of psilocybin. The RMET has 36 items, in which subjects are presented with a photograph of the eyes region of the face and must choose 1 of 4 adjectives or phrases to describe the mental state of the person pictured. A definition handout is provided at the beginning of the task and a practice item precedes the first trial.

In some embodiments, the Social Value Orientation (SVO) test is utilized to evaluate the safety and/or efficacy of psilocybin. The SVO Slider Measure has 6 primary items with 9 secondary (and optional) items. All of the items have the same general form. Each item is a resource allocation choice over a well-defined continuum of joint payoffs.

In some embodiments, after treating according to the methods of the disclosure, one or more of the subject's Social Cognition Panel Scales Score, i.e., PET, RMET, SVO, TEQ, and/or SSR score), improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Toronto Empathy Questionnaire (TEQ) is utilized to evaluate the safety and/or efficacy of psilocybin. The TEQ represents empathy as a primarily emotional process. The TEQ has exhibited good internal consistency and high test-retest reliability. The TEQ is a brief, reliable and valid instrument for the assessment of empathy. In some embodiments, after treating according to the methods of the disclosure, a subject's TEQ score increases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Scale of Social Responsibility (SSR) is utilized to evaluate the safety and/or efficacy of psilocybin. The SSR measures perceptions regarding the importance of ethics and social responsibility.

In some embodiments, the Sheehan Suicidality Tracking Scale (SSTS) is utilized to evaluate the safety and/or efficacy of psilocybin. The SSTS is a 16-item scale that assesses the seriousness of suicidality phenomena on a Likert-type scale (0-4) ranging from “not at all” (0) to “extremely”. The SSTS assesses the frequency of key phenomena and the overall time spent in suicidality.

In some embodiments, the Mini International Neuropsychiatric Interview (MINI) (version 7.0.2) is utilized to evaluate the safety and efficacy of psilocybin. The MINI is a brief structured interview for the major Axis I psychiatric disorders in DSM-5 and International Classification of Diseases-10. In some embodiments, the MINI is used to diagnose a subject with a disorder.

In some embodiments, the McLean Screening Instrument for Borderline Personality Disorder (MSIBPD) is utilized for evaluating the safety and/or efficacy of psilocybin. The MSIBPD is a useful screening tool for identifying the presence of DMS-IV borderline personality disorder.

In some embodiments, the Tellegen Absorption Scale is utilized for evaluating the safety and/or efficacy of psilocybin. The Tellegen Absorption Scale is a 34-item multidimensional measure that assesses imaginative involvement and the tendency to become mentally absorbed in everyday activities.

In some embodiments, the safety and/or efficacy of psilocybin is evaluated by physical examination. A physical examination, includes, but is not limited to, an examination of the subject's general appearance, including an examination of the skin, neck, eyes, ears, nose, throat, heart, lungs, abdomen, lymph nodes, extremities and musculoskeletal system.

In some embodiments, body weight and height of a subject are assessed. In some embodiments, body mass index is used to assess the safety and/or efficacy of psilocybin.

In some embodiments, an electrocardiogram (ECG) is utilized to evaluate the safety and/or efficacy of psilocybin. In some embodiments, a Standard 12-lead ECG is obtained.

In some embodiments, vital signs of a subject are used to evaluate safety and/or efficacy of psilocybin. Vital signs include, but are not limited to, blood pressure (BP), respiratory rate, oral body temperature and pulse. In some embodiments, blood pressure is taken after a subject has been sitting down for at least three minutes.

In some embodiments, clinical laboratory tests are utilized to evaluate the safety and/or efficacy of psilocybin. In some embodiments, the clinical laboratory tests include blood samples and/or urine samples. In some embodiments, hemoglobin, hematocrit, red blood cell count, mean corpuscular hemoglobin, mean corpuscular volume, mean corpuscular hemoglobin concentration, white blood cell count (with differential) and platelet count are measured to evaluate safety and/or efficacy of psilocybin. In some embodiments, albumin, alkaline phosphatase, alanine aminotransferase (ALT), amylase, aspartate aminotransferase (AST), bicarbonate, bilirubin (direct, indirect and total), calcium, chloride, creatine kinase, creatinine, γ-glutamyl transferase, glucose, lactate dehydrogenase, lipase, magnesium, phosphate, potassium, protein-total, sodium, blood urea nitrogen and/or uric acid are measured to evaluate the safety and/or efficacy of psilocybin.

In some embodiments, urine is tested for pregnancy and/or illicit drugs.

In some embodiments, the safety and/or efficacy of psilocybin are evaluated by measuring adverse events. Adverse events are classified as mild, moderate, or severe. A mild adverse event does not interfere in a significant manner with the subject's normal level of functioning. A moderate adverse event produces some impairment of functioning, but is not hazardous to the subject's health. A serious adverse event produces significant impairment of functioning or incapacitation and is a definite hazard to the subject's health. Adverse events may include, for example, euphoric mood, dissociative disorder, hallucination, psychotic disorder, cognitive disorder, disturbances in attention, mood alterations, psychomotor skill impairments, inappropriate affects, overdoses, and intentional product misuse. In some embodiments, serious adverse events include death, life-threatening adverse events, inpatient hospitalization or prolongation of existing hospitalization, persistent or significant disability/incapacity, and congenital anomaly/birth defect in the offspring of a subject who received psilocybin. In some embodiments, serious adverse events include intensive treatment in an emergency room or at home for allergic bronchospasm, blood dyscrasias or convulsions that do not result in inpatient hospitalization, or the development of drug dependency or drug abuse.

### Clinical Depression Evaluation

In some embodiments, the sign or symptom of depression is measured in a subject before, during, or after treatment with the methods described herein. In some embodiments, the sign or symptom of depression is measured according to a diary assessment, an assessment by clinician or caregiver, a clinical rating scale, an imaging test, or a blood of CSF test.

In some embodiments, the sign or symptom of depression in a subject is measured using a neuropsychological assessment or clinical rating scale. In some embodiments, the neuropsychological assessment or clinical rating scale is the Hamilton Depression Rating Scale, the Clinical Global Impression (CGI) Scale, the Montgomery-Åsberg Depression Rating Scale (MADRS), the Beck Depression Inventory (BDI), the Zung Self-Rating Depression Scale, the Raskin Depression Rating Scale, the Inventory of Depressive Symptomatology (IDS), the Quick Inventory of Depressive Symptomatology (QIDS), the Columbia-Suicide Severity Rating Scale, or the Suicidal Ideation Attributes Scale.

In some embodiments, the sign or symptom of depression in a subject is measured using the Hamilton Depression Rating (HAM-D) scale. The HAM-D scale is a 17-item scale that measures depression severity before, during, or after treatment. The scoring is based on 17 items and generally takes 15-20 minutes to complete the interview and score the results. Eight items are scored on a 5-point scale, ranging from 0=not present to 4=severe. Nine items are scored on a 3-point scale, ranging from 0=not present to 2=severe. A score of 10-13 indicates mild depression, a score of 14-17 indicates mild to moderate depression, and a score over 17 indicates moderate to severe depression. In some embodiments, after treatment with the methods described herein, the subject's HAM-D score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Clinical Global Impression (CGI) scale. The CGI scale is a 3-item scale that measures illness severity, global improvement or change, and therapeutic response. The CGI is rated on a 7-point scale, with the severity of illness measured using a range of responses from 1 (normal) through 7 (amongst the most severely ill subjects). In some embodiments, after treatment with the methods described herein, the subject's CGI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Montgomery-Åsberg Depression Rating Scale (MADRS). The MADRS scale is a 10-item scale that measures the core symptoms of depression. Nine of the items are based upon patient report, and 1 item is on the rater's observation during the rating interview. A score of 7 to 19 indicates mild depression, 20 to 34 indicates moderate depression, and over 34 indicates severe depression. MADRS items are rated on a 0-6 continuum with 0=no abnormality and 6=severe abnormality. In some embodiments, after treatment with the methods described herein, the subject's MADRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the subject's MADRS score decreases by at least about 50% compared to prior to treatment. In some embodiments, after treatment with the methods described herein, the subject's MADRS score decreases by between 5 and about 20 points, for example, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points and about 20 points, compared to prior to treatment. In some embodiments, after treatment with the methods described herein, the subject's MADRS score is ≤10 points (i.e., the treated subject is a MADRS remitter). In some embodiments, the subject's MADRS score is ≤10 points for at least about 3 weeks to about 12 weeks, for example, about 3 weeks, about 4 weeks, about 5 weeks, about 6 weeks, about 7 weeks, about 8 weeks, about 9 weeks, about 10 weeks, about 11 weeks and about 12 weeks, after treatment with the methods described herein.

In some embodiments, the sign or symptom of depression in a subject is measured using the Beck Depression Inventory (BDI). The BDI is a 21-item, self-report rating inventory that measures characteristic attitudes and symptoms of depression. The items are rated on 0-3 continuum with 0=no abnormality and 3=severe abnormality. A score of 17-20 indicates borderline clinical depression, a score of 21-30 indicates moderate depression, a score of 31-40 indicates severe depression, and over 40 indicates extreme depression. In some embodiments, after treatment with the methods described herein, the subject's BDI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Zung Self-Rating Depression Scale. The Zung Self-Rating Depression Scale is a 20-item self-report questionnaire that measures the psychological and somatic symptoms associated with depression. The questionnaire takes about 10 minutes to complete, and items are framed in terms of positive and negative statements. Each item is scored on a Likert scale ranging from 1 to 4. A total score is derived by summing the individual item scores, and ranges from 20 to 80. Most people with depression score between 50 and 69, while a score of 70 and above indicates severe depression. In some embodiments, after treatment with the methods described herein, the subject's Zung Self-Rating Depression score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Raskin Depression Rating Scale. The Raskin Depression Rating Scale measures baseline levels of depression and change in depression severity overtime. Each item is scored on a scale ranging from 1 to 5 with 1=not at all and 5=very much. A total score is derived by summing the individual item scores, and scores of 9 or greater represents moderate depression. In some embodiments, after treatment with the methods described herein, the subject's Raskin Depression Rating Scale score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Inventory of Depressive Symptomatology (IDS). The IDS is a 30-item inventory that measures depressive signs and symptoms. Each item is scored on a scale of 0 to 3 with 0=absence of pathology and 3=severe pathology. A total score is derived by summing the individual item scores; scores between 26-38 indicates mild depression, scores between 39-48 indicate moderate depression, and scores 49 or greater indicate severe depression. In some embodiments, after treatment with the methods described herein, the subject's IDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Quick Inventory of Depressive Symptomatology (QIDS). The QIDS is a 16-item inventory that measures depressive signs and symptoms. Each item is scored on a scale of 0 to 3 with 0=absence of pathology and 3=severe pathology. A total score is derived by summing the individual item scores; scores between 11-15 indicate moderate depression, scores between 16-20 indicate severe depression, and scores 21 or greater indicate very severe depression. In some embodiments, after treatment with the methods described herein, the subject's QIDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Young Mania Rating Scale (YMRS). The YMRS is an 11-item inventory that measures manic signs and symptoms. There are 4 items that are graded on a 0 to 8 scale, ranging from 0=absent to 8=severe. The remaining 7 items are graded on a 0 to 4 scale, ranging from 0=absent to 4=severe. A total score is derived by summing the individual item scores; scores between 9-15 indicate mild mania, scores between 16-25 indicate moderate mania, and scores 26 or greater indicate severe mania. In some embodiments, after treatment with the methods described herein, the subject's YMRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Columbia-Suicide Severity Rating Scale (C-SSRS). The C-SSRS measures the severity of suicidal ideation and behavior. The scale contains 10 binary questions (no=0 points and yes=1 point) and each question addresses a different component of the subject's suicidal ideation severity and behavior. A subject is considered to have suicidal ideation and/or behavior if they answer “yes” to any of the 10 questions. In some embodiments, after treatment with the methods described herein, the subject's C-SSRS score decreases by between about 5% and about 100%, for example, about 5% about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Suicidal Ideation Attributes Scale (SIDAS). The SIDAS measures the presence and severity of suicidal thoughts. The scale contains 5 questions measured on a 10-point scale with 0=never and 10=always. Total scores are calculated as the sum of the 5 items and range from 0 to 50. Scores of 21 or greater indicate high risk of suicidal behavior. In some embodiments, after treatment with the methods described herein, the subject's SIDAS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom in subjects with depression is measured using a Spielberger's Trait and Anxiety Inventory, a Generalized Anxiety Disorder 7-Item Scale, a Warwick-Edinburgh Mental Wellbeing Scale, a Flourishing Scale, a Snaith Hamilton Anhedonia Pleasure Scale, a Life Orientation Test, a Meaning in Life Questionnaire, a Brief Resilience Scale, a Dysfunctional Attitudes Scale, a 44-item Big Five Inventory, a Peters 21-item Delusional Inventory, an Examination of Anomalous Self-Experience, a Ruminative Responses Scale, a White Bear Suppression Inventory, a Barrett Impulsivity Scale, a Brief Experiential Avoidance Questionnaire, a Modified Tellegen Absoprtion Questionnaire, a Scale to Assess Therapeutic Relationship, Credibility/Expectancy Questionnaire, a Connectedness to Nature Scale, a Political Perspective Questionnaire, a Social Connectedness Scale, a Bech-Rafaelsen Mania Rating Scale, a Revised Santa Clara brief compassion scale, a Gratitude Questionnaire, a Short Suggestibility Scale, a Rosenberg Self-Esteem Scale, a Universality Subscale of the Spiritual Transcendence Scale, an Oxford Questionnaire on the Emotional Side-effects of Antidepressants, a Lauks Emotional Intensity Scale, Sexual Dysfunction Questionnaire, a Brief Index of Sexual Functioning for Women, a Sexual Perceptions Questionnaire, a Barnes Akathisia Rating Scale, a Work Productivity and Activity Impairment Questionnaire, a Work and Social Adjustment Scale, a Connectedness Questionnaire, a Standard Assessment of Personality, a Positive and Negative Syndrome Scale, a Mastery Insight Scale, a Self-Reflection and Insight Scale, a Psychological Insight Scale, a Metaphysical Beliefs Questionnaire, a Spiritual Bypassing Scale, an Adverse Childhood Experience Questionnaire, a Therapeutic Music Experience Questionnaire, a Setting Questionnaire, an Absorption in Music Scale, a Psychedelic Predictor Scale, a Surrender Scale, a EuroQOL-5 Dimension-3 Level Scale, or any combinations thereof. In some embodiments, after treatment with the methods described herein, the sign or symptom of depression measured using any of the assessments listed above decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using an imaging test before, during, or after treatment with the methods described herein. In some embodiments, the imaging test is a CT scan. In some embodiments, the imaging test is a functional MRI scan. In some embodiments, the functional MRI scan measures the blood oxygen level-dependent (BOLD) response as an indicator of brain activity and/or functional connectivity. In some embodiments, the BOLD response is measured in the subject at resting state, in response to emotional faces, or in response to music. In some embodiments, after treatment with the methods described herein, the BOLD response in a region of the brain increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In other embodiments, after treatment with the methods described herein, the BOLD response in a region of the brain decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression is measured using a marker for depression in the blood or cerebral spinal fluid. In some embodiments, the marker for depression is measured in the subject before, during, or after treatment with the methods or compositions described herein. In some embodiments, the marker for depression is red blood cell folate, serum folate, vitamin B12, plasma homocysteine, serum methylfolate, and/or testing for one or more of brain-derived neurotrophic factor (BDNF) Val66Met, bone morphogenetic protein rs41271330, and/or 5-HTTLPR polymorphisms. In some embodiments, the marker for depression decreases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment. In other embodiments, the marker for depression increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

### NUMBERED EMBODIMENTS OF THE DISCLOSURE

In addition to the disclosure above, the Examples below, and the appended claims, the disclosure sets forth the following numbered embodiments.

1. A method of treating treatment-resistant depression with psilocybin in a subject that did not respond to a first dose of psilocybin, comprising administering a second dose of psilocybin 2 days to about 3 weeks after administering the first dose of psilocybin.

2. A method of treating treatment-resistant depression in a subject in need thereof, comprising
administering a first dose of psilocybin to the subject;
measuring the subject's depressive symptoms using a clinical depression evaluation after the first dose of psilocybin;
identifying the subject as a non-responder to the first dose of psilocybin; and
administering a second dose of psilocybin to the subject 2 days to about 3 weeks after administering the first dose.

3. The method of embodiment 1 or 2, wherein the subject exhibits no or substantially no reduction in symptoms of depression after administering the first dose of psilocybin.

4. The method of embodiment 1, comprising identifying the subject as non-responsive to the first dose of psilocybin using a clinical depression rating scale.

5. The method of embodiment 2 or 4, wherein the clinical depression evaluation is Montgomery-Åsberg Depression Rating Scale (MADRS).

6. The method of any one of embodiments 2-5, wherein the subject that is non-responsive has a change in baseline in a MADRS of about-10 to 0 or less than 50% after administering the first dose of psilocybin.

7. Th method of embodiment 6, wherein the subject has a change in baseline in the MADRS of −10, −9, −8, −7, −6, −5, −4, −3, −2, or −1 after administering the first dose of psilocybin.

8. The method of embodiment 6 or 7, wherein the change in baseline in the MADRS is measured on Day 2, Day 3, Day 4, Day 5, or Day 6 after administering the first dose of psilocybin, or 1 week, 2 weeks, or 3 weeks after administering the first dose of psilocybin.

9. The method of any one of embodiments 1-7, wherein the subject is responsive to the second dose of psilocybin.

10. The method of any one of embodiments 1-9, wherein the subject exhibits a reduction in symptoms of depression after administering the second dose of psilocybin.

11. The method of any one of embodiments 1-10, comprising identifying the subject as responsive to the second dose of psilocybin using a clinical depression rating scale.

12. The method of embodiment 11, wherein the clinical depression rating scale is MADRS, and the subject has a change in baseline in the MADRS of −10 to −30 after administering the second dose of psilocybin.

13. The method of embodiment 12, wherein the subject has a change in baseline in the MADRS of −11, −12, −13, −14, −15, −16, −17, −18, −19, −20, −21, −22, −23, −24, or −25, after administering the second dose of psilocybin.

14. The method of embodiment 12 or 13, wherein the change in baseline in the MADRS is measured on Day 2, Day 3, Day 4, Day 5, or Day 6 after administering the second dose of psilocybin, or 1 week, 2 weeks, or 3 weeks after administering the second dose of psilocybin.

15. The method of any one of embodiments 1-14, wherein the first dose comprises 1 mg of psilocybin and second dose comprises 1 mg of psilocybin.

16. The method of any one of embodiments 1-14, wherein the first dose comprises 10 mg of psilocybin and second dose comprises 10 mg of psilocybin.

17. The method of any one of embodiments 1-14, wherein the first dose comprises 25 mg of psilocybin and second dose comprises 25 mg of psilocybin.

18. The method of any one of embodiments 1-17, wherein the psilocybin is administered to the subject in a pharmaceutical composition.

19. The method of embodiment 18, wherein the pharmaceutical composition comprises psilocybin, pregelatinized starch, and sodium stearyl fumarate.

20. The method of embodiment 18 or 19, wherein the pharmaceutical composition comprises about 1%-10%, by weight, psilocybin.

21. The method of any one of embodiments 18-20, wherein the pharmaceutical composition comprises about 85-99%, by weight, pregelatinized starch.

22. The method of any one of embodiments 18-21, wherein the pharmaceutical composition comprises about 0.5%-2%, by weight, sodium stearyl fumarate.

23. The method of any one of embodiments 1-22, wherein the psilocybin is crystalline psilocybin characterized by XRPD peaks at 11.5±0.1, 12.0±0.1, 14.5±0.1, 17.5±0.1 and 19.7±0.1°2θ.

24. The method of embodiment 23, wherein the crystalline psilocybin has a chemical purity of greater than 97% as determined by HPLC analysis.

25. The method of embodiment 24, wherein the crystalline psilocybin has no single impurity of greater than 2%.

26. A method of treating treatment-resistant depression in a subject that responded to a first dose of psilocybin, comprising administering a second dose at least about 20 weeks after administering the first dose.

27. The method of embodiment 26, wherein the second dose is administered 20 to 28 weeks after the first dose.

28. The method of embodiment 26, wherein the second dose is administered at least about 20 weeks, about 25 weeks, about 27 weeks, about 28 weeks, about 30 weeks, about 35 weeks, about 40 weeks, or about 45 weeks after the first dose.

29. The method of any one of embodiments 26-28, wherein the subject experiences a reduction in symptoms of depression after administering the first dose of psilocybin.

30. The method of any one of embodiments 26-29, further comprising measuring the subject's depressive symptoms after administering the first dose of psilocybin using a clinical depression rating scale.

31. The method of embodiment 30, wherein the clinical depression rating scale is MADRS.

32. The method of embodiment 32, wherein the subject has a change in baseline in the MADRS of −10 to −30 after administering the first dose if psilocybin.

33. The method of embodiment 32, wherein the subject has a change in baseline in the MADRS of −11, −12, −13, −14, −15, −16, −17, −18, −19, −20, −21, −22, −23, −24, or −25, after administering the first dose of psilocybin.

34. The method of embodiment 32 or 33, wherein the change in baseline is the MADRS is measured on Day 2, Day 3, Day 4, Day 5, or Day 6 after administering the first dose of psilocybin or 1 week, 5 weeks, 10 weeks, 12 weeks, 15 weeks, 16 weeks, 20 weeks, 24 weeks, 25 weeks, 28 weeks after administering the first dose of psilocybin, or up to 1 day before administering the second dose.

35. The method of any one of embodiments 26-34, wherein the subject continues to experience no or substantially no increase in symptoms of depression after administration of the second dose of psilocybin.

36. The method of embodiment 35, wherein the subject maintains a change in baseline in the MADRS of −10 to −30 after administering the second dose.

37. The method of embodiment 36, wherein the subject maintains a change in baseline in the MADRS of −11, −12, −13, −14, −15, −16, −17, −18, −19, −20, −21, −22, −23, −24, −25, −26, −27, −28, −29, or −30 after administering the second dose.

38. The method of any one of embodiments 26-37, wherein the first dose comprises 1 mg of psilocybin and second dose comprises 1 mg of psilocybin.

39. The method of any one of embodiments 27-37, wherein the first dose comprises 10 mg of psilocybin and second dose comprises 10 mg of psilocybin.

40. The method of any one of embodiments 27-37, wherein the first dose comprises 25 mg of psilocybin and second dose comprises 25 mg of psilocybin.

41. The method of any one of embodiments 26-40, wherein the psilocybin is administered to the subject in a pharmaceutical composition.

42. The method of embodiment 41, wherein the pharmaceutical composition comprises psilocybin, pregelatinized starch, and sodium stearyl fumarate.

43. The method of embodiment 41 or 42, wherein the pharmaceutical composition comprises about 1%-10%, by weight, psilocybin.

44. The method of any one of embodiments 41-43, wherein the pharmaceutical composition comprises about 85-99%, by weight, pregelatinized starch.

45. The method of any one of embodiments 41-44, wherein the pharmaceutical composition comprises about 0.5%-2%, by weight, sodium stearyl fumarate.

46. The method of any one of embodiments 26-45, wherein the psilocybin is crystalline psilocybin characterized by XRPD peaks at 11.5±0.1, 12.0±0.1, 14.5±0.1, 17.5±0.1 and 19.7±0.1°2θ.

47. The method of embodiment 46, wherein the crystalline psilocybin has a chemical purity of greater than 97% as determined by HPLC analysis.

48. The method of embodiment 46 or 47, wherein the crystalline psilocybin has no single impurity of greater than 2%.

49. The method of embodiment 1 or 26, wherein the first dose of psilocybin comprises about 1-25 mg of psilocybin.

50. The method of embodiment 1 or 26, wherein the second dose of psilocybin comprises about 1-25 mg of psilocybin.

51. A method of treating treatment-resistant depression in a subject in need thereof, comprising orally administering to the subject about 25 mg of psilocybin once daily.

52. The method of embodiment 51, comprising measuring the subject's depressive symptoms using a clinical depression evaluation at baseline (before administering 25 mg of psilocybin).

53. The method of embodiment 51 or 52, comprising measuring the subject's depressive symptoms using a clinical depression evaluation after administering 25 mg of psilocybin.

54. The method of any one of embodiments 51-53, wherein the subject shows an improvement in depressive symptoms compared to baseline.

55. The method of any one of embodiments 53-54, wherein the clinical depression evaluation is measured on Day 2 after administering 25 mg of psilocybin.

56. The method of embodiment 55, wherein the clinical depression evaluation is further measured on one or more of week 1, week 3, week 6, week 9, week 12, week 16, week 20, week 24, week 28, week 40, or week 52.

57. The method of any one of embodiments 51-56, wherein the subject shows an improvement clinical depression evaluation for at least 28 weeks.

58. The method of any one of embodiments 51-57, wherein the clinical depression evaluation is MADRS.

59. The method of embodiment 58, wherein the subject has a change in baseline in MARDS of about-11 or more, or greater than 50% on Day 2 after administering 25 mg of psilocybin.

58. The method of embodiment 59, wherein the subject maintains a change in baseline in MADRS of about-11 or more, or greater than 50% for at least 20 weeks after administering 25 mg of psilocybin.

59. The method of embodiment 59, wherein the subject maintains a change in baseline in MADRS of about-11 or more, or greater than 50% for at least 28 weeks after administering 25 mg of psilocybin.

60. The method of embodiment 51-59, wherein the subject is not administered a second dose of psilocybin after receiving 25 mg of psilocybin.

61. The method of any one of embodiments 51-60, wherein the psilocybin is administered to the subject in a pharmaceutical composition.

62. The method of embodiment 61, wherein the pharmaceutical composition comprises psilocybin, pregelatinized starch, and sodium stearyl fumarate.

63. The method of embodiment 61 or 62, wherein the pharmaceutical composition comprises about 1%-10%, by weight, psilocybin.

64. The method of any one of embodiments 61-63, wherein the pharmaceutical composition comprises about 85-99%, by weight, pregelatinized starch.

65. The method of any one of embodiments 61-64, wherein the pharmaceutical composition comprises about 0.5%-2%, by weight, sodium stearyl fumarate.

66. The method of any one of embodiments 61-65, wherein the psilocybin is crystalline psilocybin characterized by XRPD peaks at 11.5±0.1, 12.0±0.1, 14.5±0.1, 17.5±0.1 and 19.7±0.1°2θ.

67. The method of embodiment 66, wherein the crystalline psilocybin has a chemical purity of greater than 97% as determined by HPLC analysis.

68. The method of embodiment 67, wherein the crystalline psilocybin has no single impurity of greater than 2%.

69. The method of embodiment 54, wherein the subject exhibits no or substantially no symptoms of depression after administering 25 mg of psilocybin.

70. The method of embodiment 51-59, wherein the subject does not experience a depressive event for at least about 27 weeks after administering 25 mg of psilocybin.

71. The method of embodiment 70, wherein the depressive event comprise initiation of new antidepressant treatment (first new antidepressant treatment in time period only); hospitalization due to depression/suicidality; suicide attempt, prevention of an imminent suicide attempt, or completed suicide; increased suicidality measured by worsening on MADRS item 10; active suicidal ideation measured by the C-SSRS; MADRS worsening: or discontinuation for adverse event or lack of efficacy.

### Examples

The following examples, which are included herein for illustration purposes only, are not intended to be limiting.

### Example 1. Formulation Development

Table 1 shows the capsule formulations of psilocybin developed for clinical use. These capsule formulations were optimized to enhance flow, blend uniformity, content uniformity and dissolution as described in WIPO Patent Appln. Pub. No. 2022/207746, the entire contents of which are incorporated herein by reference.

TABLE 1
Unit Formula for Psilocybin Capsules

| | 1 mg | 1 mg | 5 mg | 5 mg | 25 mg | 25 mg |
|---|---|---|---|---|---|---|
| Excipient | (%) | (g) | (%) | (g) | (%) | (g) |
| Psilocybin (COM | 1.0 | 7.0 | 5.0 | 35.0 | 10.0 | 70.0 |
| 360) | | | | | | |
| Pregelatinized | 98.0 | 686 | 94.0 | 658 | 89.0 | 623.0 |
| starch (Starcap) | | | | | | |
| Sodium Stearyl | 1.0 | 7.0 | 1.0 | 7.0 | 1.0 | 7.0 |
| Fumarate (Pruv) | | | | | | |
| Capsule, HPMC, | 1 unit | Size 2* | 1 unit | Size 2* | 1 unit | Size 0** |
| white opaque | | | | | | |
| TOTAL | 100.0 | 700.0 | 100.0 | 700.0 | 100.0 | 700.0 |

*fill weight 100 mg,
**fill weight 250 mg

### Example 2. Clinical Study Examining Psilocybin for the Treatment of Treatment-Resistant Depression

Studies with more rigorous designs are needed to better the therapeutic potential of psilocybin in treatment-resistant depression (TRD). The aim of this study was to assess the effectiveness of 3 different doses of psilocybin (1 mg, 10 mg, and 25 mg) in TRD.

This was a Phase 2b, international multicentre, randomized, fixed dose, double-blind trial. The study population included adult men and women, 18 years of age and older, with TRD. Subjects with TRD are defined as those who meet the Diagnostic and Statistical Manual of Mental Disorders (5th Edition; DSM-5) diagnostic criteria for single or recurrent episode of major depressive disorder (MDD) without psychotic features which have failed to respond to an adequate dose and duration of 2, 3, or 4 pharmacological treatments for the current episode; if single episode MDD, the duration of the current episode must be at least 3 months but not more than 2 years. Augmentation counts as a second treatment, provided it was approved for the adjunctive treatment of MDD in that country.

Subjects were outpatients and were recruited primarily from general practitioners and specialized psychiatric services.

The majority of subjects had no prior exposure to psilocybin or so-called magic mushrooms; however, to reflect the prevalence of experience in general population, we allowed up to 10% of subjects with prior recreational experience with psilocybin or magic mushrooms (6% of study participants had prior recreational experience with psilocybin or magic mushrooms). Past exposure to psilocybin was be more than 12 months prior to screening and not during the current depressive episode. This was constrained by the centralized randomization process, Interactive Web-based Response System.

Subjects were assessed for their eligibility with the Mini International Neuropsychiatric Interview version 7.0.2 (MINI 7.0.2), the Hamilton Depression Rating Scale (HAM-D-17), the Massachusetts General Hospital Antidepressant Treatment History Response Questionnaire (MGH-ATRQ), the Columbia-Suicide Severity Rating Scale (C-SSRS), and McLean Screening Instrument for Borderline Personality Disorder (MSI-BPD). Those who met the eligibility criteria entered the screening period which lasted between 3 and 6 weeks. At the initial screening visit (Visit 1), the subject was evaluated with the 16-Item Quick Inventory of Depressive Symptomatology-Self Report (QIDS-SR-16), and the Adult Self Report Scale (ASRS). Additionally, a medical history, an electrocardiogram (ECG), blood tests, and vital signs were obtained.

Subjects taking antidepressant medications tapered their use at least 2 weeks prior to baseline (Visit 2). Eligible subjects were invited for a screening visit (Visit 1a). Visit 1a is the point at which the subject begins tapering off their antidepressant and/or antipsychotic medications, if appropriate. The subject must complete the taper within the first 4 weeks of this period, prior to 2 weeks completely off antidepressant and/or antipsychotic medications, before baseline Visit 2. The tapering period used in the study was set at the industry standard for depression trials.

All subjects were evaluated for safety at the clinic weekly for a minimum of 3 weeks prior to psilocybin administration to ensure safe discontinuation of current antidepressant therapy required by the protocol. Subjects' companions (friend or family member) were educated about the signs of worsening of depression and suicidality, and instructed on ways to contact the study team in case of significant worsening of depression. Any safety assessment visits during the screening period were called Visit 1a, Visit 1b, etc. During these visits, the C-SSRS and any changes in medications since the previous visit were obtained in addition to other assessments at the study clinician's discretion.

The subject met with a therapist for a minimum of 3 visits during screening. These are called safety sessions and covered what to expect during the psilocybin session. The therapist and the subject reviewed psychoeducational materials provided at the time of enrolment.

The day before the psilocybin session, the subjects underwent a baseline assessment (3 to 6 weeks after initial screening [Visit 1]) that consisted of the HAM-D-17, MADRS, QIDS-SR-16, C-SSRS, SDS, GAD-7, DSST, EQ-5D-3L (administered to both subject and caregiver [the latter was not mandatory]), WSAS, vital signs, urinalysis, urine drug screen, and urine pregnancy test (only for women of childbearing potential). Both the therapist and the subject were asked to fill out a therapeutic alliance evaluation questionnaire, STAR-C and STAR-P, respectively. After baseline data was entered into EDC, the CAT team completed a final review to ensure the subject's continued eligibility. Subjects were not progressed to Visit 3 until this approval is received.

The psilocybin administration session (Visit 3, Day 0) lasted approximately 6 h and were supported by a trained therapist. Some psilocybin sessions were video recorded for training and adherence monitoring. After the acute effects of the psilocybin pass, subjects were evaluated for safety and accompanied home. On Day 1 (Visit 4), the day following psilocybin administration, subjects were seen in person for a safety check, assessment of suicidality, and to discuss their experience during the psilocybin session. All sessions between the therapist and the subject may be audio recorded for adherence monitoring and quality assurance. Audio and video recording of the sessions were subject to subject consent. Subjects who do not consent to either or all recordings were not be excluded from the study.

All subjects were asked to remain off their antidepressant medications for at least 3 weeks following the psilocybin session until the primary endpoint assessment, or longer. Rescue medications were allowed and described in the Rescue Medications section below. Subjects who restarted their antidepressant medications during the first 3 weeks after the psilocybin treatment administration were assessed for reasons of resuming their medications and followed until 12 weeks post-psilocybin administration.

The treatment period was determined the optimal therapeutic dose; 233 subjects were randomised in an approximately 1:1:1 ratio to receive 1 mg psilocybin (79 subjects), 10 mg psilocybin (75 subjects), or 25 mg psilocybin (79 subjects). Average patient baseline MADRS values were 31.9 in the 25 mg group, 33.0 in the 10 mg group, and 32.7 in the 1 mg group. This represents moderate to severe depression (≥31=cut-off for severe depression based on MADRS).

TABLE 2
Demographics of the Subjects

| | | | | |
|---|---|---|---|---|
| | 25 mg | 10 mg | 1 mg | |
| | psilocybin | psilocybin | psilocybin | Overall |
| Parameter | (n = 79) | (n = 75) | (n = 79) | (n = 233) |

Sex, n (%)

| | | | | |
|---|---|---|---|---|
| Male | 35 (44.3) | 34 (45.3) | 43 (54.4) | 112 (48.1) |
| Female | 44 (55.7) | 41 (54.7) | 36 (45.6) | 121 (51.9) |

Ethnicity, n (%)

| | | | | |
|---|---|---|---|---|
| White | 70 (88.6) | 72 (96.0) | 73 (92.4) | 215 (92.3) |
| Black or | 4 (5.1) | 0 | 1 (1.3) | 5 (2.1) |
| African | | | | |
| American | | | | |
| Asian | 4 (5.1) | 3 (4.0) | 5 (6.3) | 12 (5.2) |
| Other | 1 (1.3) | 0 | 0 | 1 (0.4) |

Age at screening, years

| | | | | |
|---|---|---|---|---|
| Mean (SD) | 40.2 (12.19) | 40.6 (12.76) | 38.7 (11.71) | 39.8 (12.19) |
| Min, Max | 18, 69 | 20, 67 | 19, 72 | 18, 72 |
| 18-34 | n (%) | 24 (30.4) | 29 (38.7) | 31 (39.2) |
| 35-64 | n (%) | 52 (65.8) | 44 (58.7) | 46 (58.2) |
| 65-84 | n (%) | 3 (3.8) | 2 (2.7) | 2 (2.5) |
| >84 | n (%) | 0 | 0 | 0 |

BMI

| | | | | |
|---|---|---|---|---|
| Mean (SD) | 26.52 (6.134) | 28.26 (8.203) | 27.26 (6.025) | 27.34 (6.858) |
| Min, Max | 17.4, 45.3 | 17.4, 49.1 | 17.3, 48.4 | 17.3, 49.1 |

Prior psilocybin experience n (%)

| | | | | |
|---|---|---|---|---|
| Yes | 5 (6.3) | 5 (6.7) | 4 (5.1) | 14 (6.0) |
| No | 74 (93.7) | 70 (93.3) | 75 (94.9) | 219 (94.0) |

Subjects were seen at the clinic for screening (Visit 1, plus a minimum of 3 safety visits), baseline (Visit 2, day-1), day 0 (Visit 3, Dosing), day 1 (Visit 4), Week 1 (Visit 5), Week 2 (Visit 6), Week 3 (Visit 7), and Week 12 (Visit 10). Subjects were contacted for follow-up at Week 6 (Visit 8) and Week 9 (Visit 9). The MADRS were done by telephone and the other assessments were done electronically. Subjects were seen at the clinic for safety visits between the initial screening (Visit 1) and the baseline (Visit 2) visit, and the visits were labelled Visit 1a, Visit 1b, Visit 1c, etc.

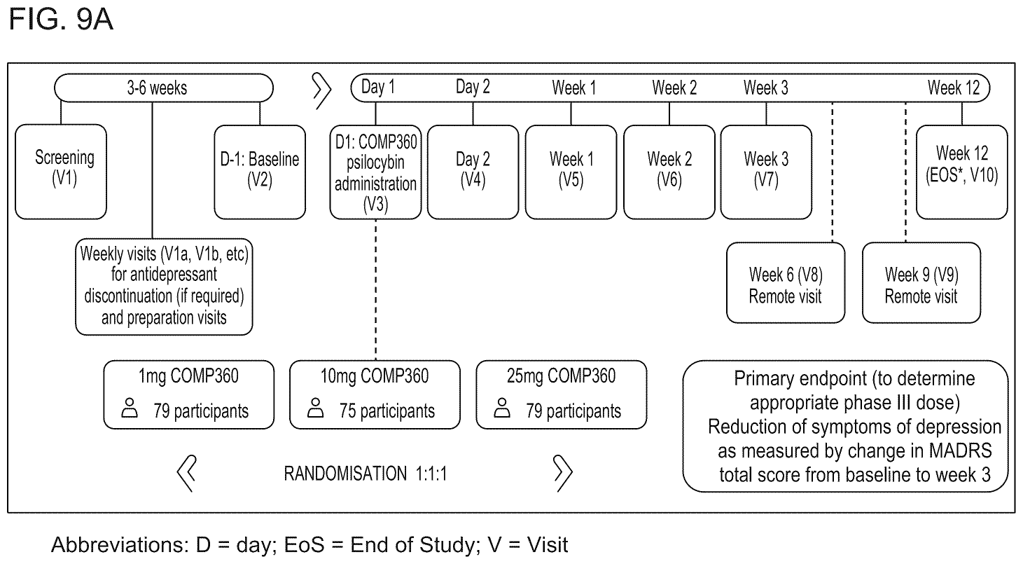

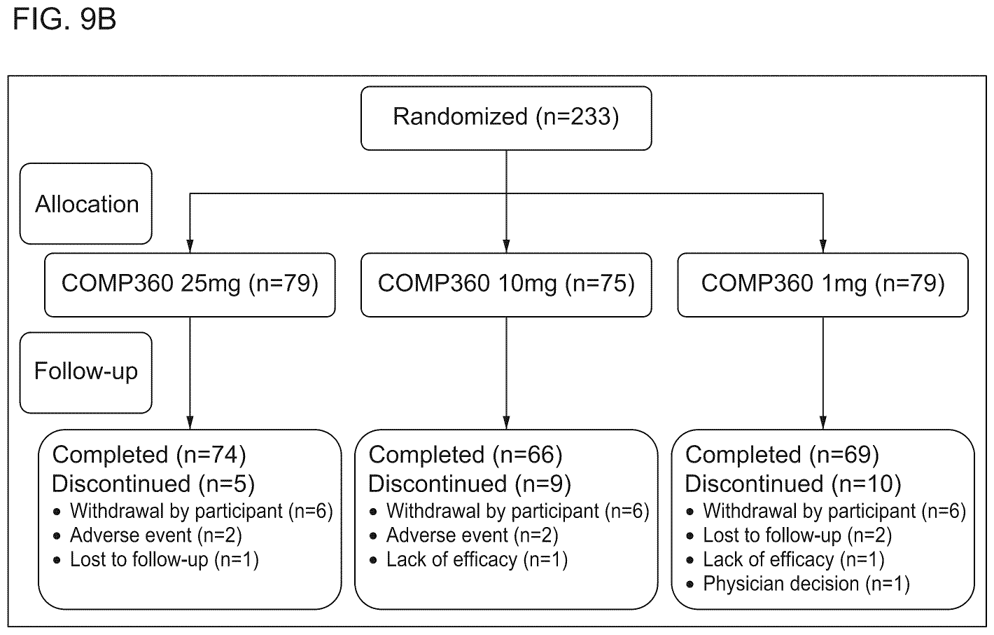

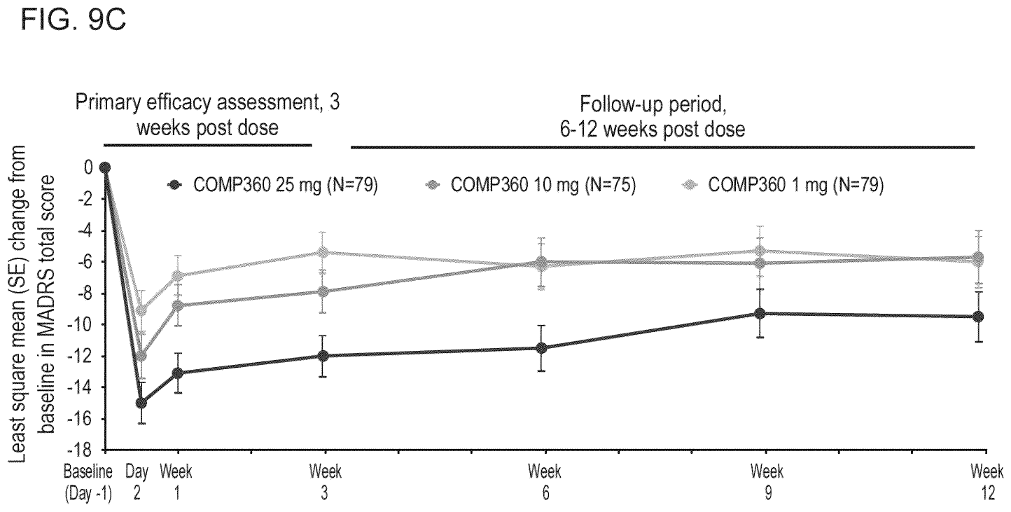

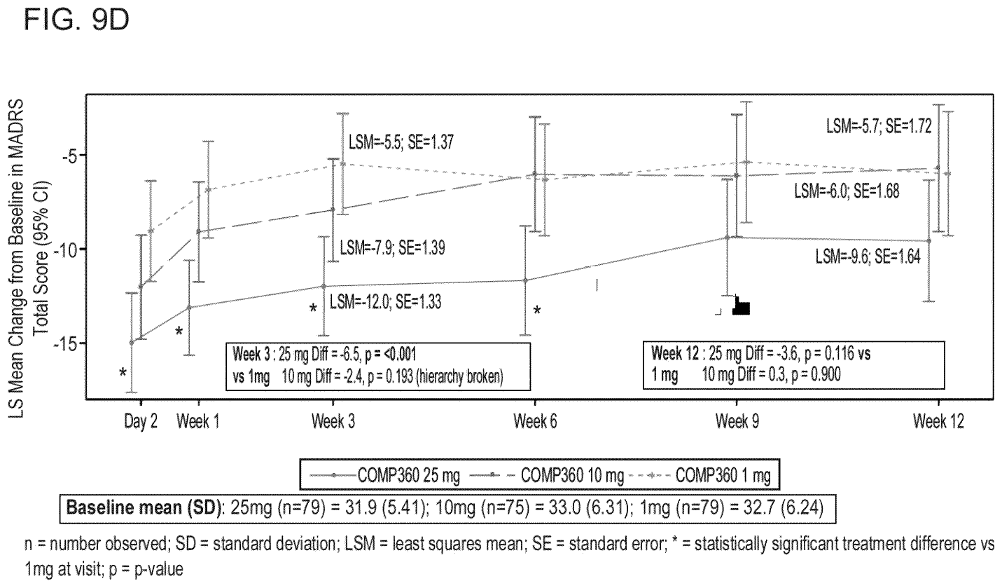

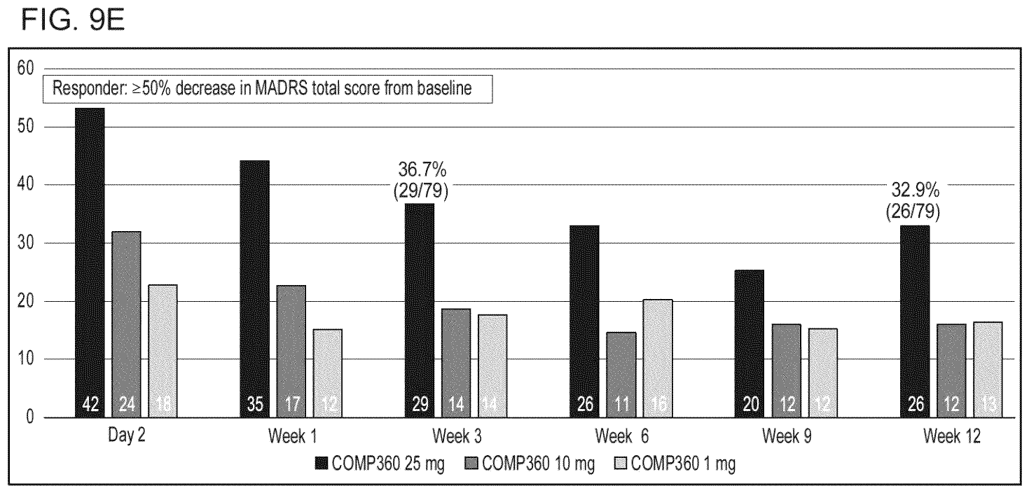

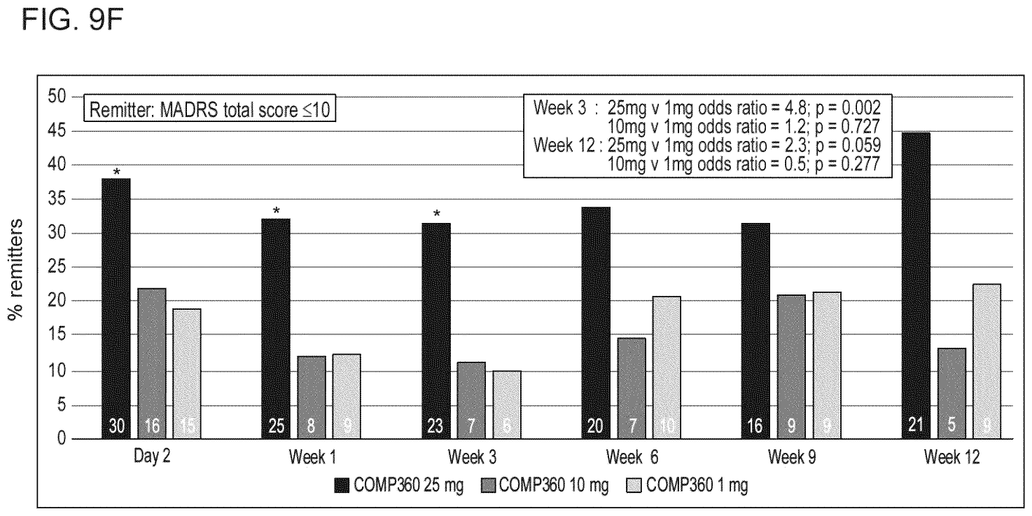

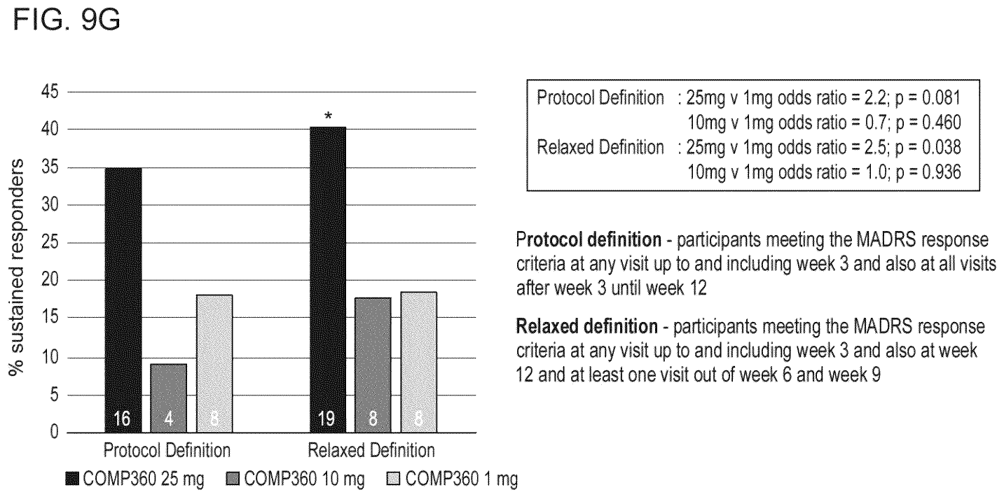

The study schematic is shown in FIG. 9A and the schedule of assessments are presented in Table 3A below.

TABLE 3A
Schedule of Assessments

| | 3-6 weeks | | | | | | | | | |
|---|---|---|---|---|---|---|---|---|---|---|
| | prior to | | | Time Post Psilocybin Session | | | | | | |
| | Baseline | | Psilocybin | | | | | | | Week 12 |
| Screen | Screening | Baseline | Session | | Week 1 | Week 2 | Week 3 | Week 6 | Week 9 | Day 84 |
| Visit² | Period | (Day −1) | (Day 0) | Day 1 | Day 7 | Day 14 | Day 21 | Day 42 | Day 63 | (ET)³ |
| Visit | | | | | | | | | | |
| 1 | 1a, 1b, etc. | 2 | 3 | 4 | 5 | 6 | 7 | 8 | 9 | 10 |
| Location of Visit¹ | | | | | | | | | | |
| Clinic | Clinic | Clinic | Clinic | Clinic | Clinic | Clinic | Clinic | Remote | Remote | Clinic |

Allowable Window

| | | | | | | | | |
|---|---|---|---|---|---|---|---|---|
| Weekly | ≤7 days | none | ±1 day | ±1 day | ±1 day | ±3 days | ±3 days | ±7 days |

Clinic Assessments and Procedures

| | | | | | | | | | | | |
|---|---|---|---|---|---|---|---|---|---|---|---|
| Informed Consent | □ | | | | | | | | | | |
| Medical History | □ | | □ | | | | | | | | |
| Inclusion/exclusion | □ | | □ | | | | | | | | |
| Criteria | | | | | | | | | | | |
| MINI | □ | | | | | | | | | | |
| HAM-D-17 | □ | | □ | | | | | | | | |
| MGH-ATRQ | □ | | | | | | | | | | |
| STAR-C | | | □ | | | | | | | | |
| C-SSRS4 | □ | □ | □ | □ | □ | □ | □ | □ | □ | □ | □ |
| Vital signs | □ | | □ | □ | □ | | | | | | |
| Weight | □ | | | | | | | | | | |
| Height | □ | | | | | | | | | | |
| ECG | □ | | | | □ | | | | | | |
| Clinical laboratory | □ | | | | □ | | | □ | | | |
| tests and biomarker⁵ | | | | | | | | | | | |
| Urinalysis⁵ | □ | | | | □ | | | | | | |
| Urine Drug Screen⁵ | □ | | □ | | | | | | | | |
| Urine Pregnancy | □ | | □ | | | | | | | | |
| Test⁶ | | | | | | | | | | | |
| Documentation of | □ | | | | | | | | | | |
| Birth Control to be | | | | | | | | | | | |
| used⁷ | | | | | | | | | | | |
| 2a polymorphism | □ | | | | | | | | | | |
| (optional) | | | | | | | | | | | |
| Activate/deactivate | □ | | | | | | | | | | □ |
| Mindstrong | | | | | | | | | | | |
| (optional) | | | | | | | | | | | |
| Provide access to | □ | □¹⁰ | | | | | | | | | |
| psychoeducational | | | | | | | | | | | |
| material (Longboat) | | | | | | | | | | | |
| Psilocybin dose | | | | □¹¹ | | | | | | | |
| Prior/Concomitant | □ | □ | □ | □ | □ | □ | □ | □ | □ | □ | □ |
| Medications | | | | | | | | | | | |
| AE/SAE | | | | □ | □ | □ | □ | □ | □ | □ | □ |
| Randomization | | | □ | | | | | | | | |

Subject Completed Assessments

| | | | | | | | | | | |
|---|---|---|---|---|---|---|---|---|---|---|
| 5D-ASC | | | □⁸ | | | | | | | |
| ASRS | □ | | | | | | | | | |
| DSST | | □ | | □ | | | □ | | | □ |
| EBI | | | | □ | | | | | | |
| EQ-5D-3L⁹ | | □ | | | | | □ | | | □ |
| GAD-7 | | □ | | | | | □ | | | □ |
| MSI-BPD | □ | | | | | | | | | |
| PANAS | | □ | | □ | | | □ | | | |
| QIDS-SR-16 | □ | □ | □ | □ | □ | □ | □ | □ | □ | □ |
| SDS | | □ | | | | | □ | | | □ |
| STAR-P | | □ | | | | | | | | |
| WSAS | | □ | | | | | □ | | | □ |

Remote-rater Assessment

| | | | | | | | |
|---|---|---|---|---|---|---|---|
| MADRS | □ | □ | □ | □ | □ | □ | □ |

Abbreviations:
5D-ASC, Five Dimension Altered States of Consciousness Questionnaire;
AE, adverse event;
ASRS, Adult Self-Report Scale;
CRP, C-reactive protein;
C-SSRS, Columbia-Suicide Severity Rating Scale;
DSM-5 Diagnostic and Statistical Manual of Mental Disorders (5th edition);
DSST, Digit Symbol Substitution Test;
EBI, Emotional Breakthrough Inventory;
ECG, electrocardiogram;
EQ-5D, EuroQoL 5-dimension;
ET, early termination;
GAD-7, Generalized Anxiety Disorder 7;
h, hour(s);
HAM-D-17, Hamilton Depression Rating Scale;
MADRS, Montgomery-Asberg Depression Scale;
MGH-ATRQ, Massachusetts General Hospital Antidepressant Treatment History Questionnaire;
MINI, Mini International Neuropsychiatric Interview;
MSI-BPD, McLean Screening Instrument for Borderline Personality Disorder;
PANAS, Positive and Negative Affect Schedule;
QIDS-SR-16, Quick Inventory of Depressive Symptomatology-Self-rated;
SAE, serious adverse event;
SDS, Sheehan Disability Scale;
STAR-C, Scale to Assess the Therapeutic Relationship-Clinician;
STAR-P, Scale to Assess the Therapeutic Relationship-Patient;
UK, United Kingdom;
VAS, visual analogue scale;
WSAS, Work and Social Adjustment Scale
¹On site clinic visits; visits allowed remotely had the MADRS performed by telephone and other assessments were done electronically.
²If additional visits were needed to ensure adequate time for discontinuation of prior antidepressant therapy, visits should occur weekly prior to the psilocybin session (V3). At subsequent screening period visits (V1a, V1b, etc.), medications taken and any changes in medications since the previous visit and C-SSRS were obtained, in addition, to other assessments at the study clinician’s discretion. Assessments were performed over several days, but all scales should be completed on the same day.
4The “Last 12 Months” version were administered at Screening and the “Since Last Visit” version were administered at all other visits.
⁵See Section 7.2.4 for complete list of required testsperformed.
⁶For women of child-bearing potential only.
⁷Site documented method of contraception agreed to be used by each subject.
⁸Administered immediately after the psilocybin session.
⁹The EQ-5D-3L were administered to both the subject and their caregiver (latter is not mandatory).
¹⁰Blood pressure Longboat access was granted at V1 or V1a at the study team’s discretion.
¹¹After baseline data was entered into EDC the CAT team completed a final review to ensure the subject’s continued eligibility. Subjects cannot complete psilocybin dose at V3 until this approval was received.
¹²Screening period visits were not be initiated until initial MM and CAT approval was received. Screening visits occured weekly 3-6 weeks prior to Baseline, number of visits was determined by the length of the subject’s taper off antidepressant medication.
Instruments (all measures below will be captured electronically):
5D-ASC—The Five Dimension Altered States of Consciousness Questionnaire measures the acute drug effects.
ASRS—The Adult Self-Report Scale is a tool to screen for attention deficit hyperactivity disorder. The 6-item screener version was administered.
C-SSRS—Columbia-Suicide Severity Rating Scale assesses treatment-emergent suicidal thoughts. This scale was administered prior to dosing, if possible.
DSST—The Digit Symbol Substitution Test is a measure of cognition. It consists of a number of digit-symbol pairs, and requires individuals to note the corresponding symbol for a given digit under time-limited conditions. This test was administered at approximately the same time of day each time to minimize the impact of diurnal variation on the results
EBI—The Emotional Breakthrough Inventory is an 8-item brief measure intended to index the degree to which an individual experiences his/her emotion during a psilocybin session. It is a VAS style scale, typically with units from 0 to 100. It is typically rated within 24 h of a psychedelic experience and ideally within 5 h of the ‘end’ of the psychedelic experience or once acute drug effects have significantly subsided.
EQ-5D-3L—The 3-level EQ-5D version (EQ-5D-3L) was introduced by the EuroQoL Group in 1990. The EQ-5D-3L essentially consists of 2 pages: the EQ-5D-3L descriptive system and the EQ visual analogue scale (EQ VAS).
HAM-D-17—The 17-item Hamilton Depression Rating scale measure the degree of symptom severity in depressed patients.
GAD-7—The Generalized Anxiety Disorder scale is a 7-item subject completed scale to assess anxiety in a subject.
MADRS—the Montgomery-Asberg Depression Scale is a clinician rated outcome measure to assess a subject’s level of depression.
MSI-BPD—The McLean Screening Instrument for Borderline Personality Disorder is a self-reporting screening tool to determine the presence of DSM-5 borderline personality disorder.
PANAS—Positive and Negative Affect Schedule measures the acute emotional drug effects.
QIDS-SR-16—Quick Inventory of Depressive Symptomatology scale is a subject-rated scale to assess their depression.
SDS—The Sheehan Disability Scale is a patient-reported outcome measure used to assess functional impairment and associated disability.
STAR-C and STAR-P—Scale to Assess the Therapeutic Relationship completed by the clinician (STAR-C) and the patient (STAR-P) to assess the therapeutic relationship in community psychiatry with good psychometric properties suitable for both research and routine care.
WSAS—Work and Social Adjustment Scale is a self-report scale used to assess psychosocial functioning and to predict durability of response to antidepressant treatment.

### Study Objectives

The primary objective of this study was to evaluate the efficacy of psilocybin (25 mg or 10 mg) compared to 1 mg, administered under supportive conditions to adult subjects with TRD, in improving depressive symptoms, as assessed by the change in the Montgomery-Asberg Depression Rating Scale (MADRS) total score from baseline. Baseline is defined as the assessment score obtained on Day-1. The primary time point is week 3; this variable was analyzed for the change from Baseline to day 1, and weeks 1, 3, 6, 9, and 12 of the study.

Additional objectives of this study were to assess the efficacy of psilocybin compared to 1 mg psilocybin on the:
(a) proportion of subjects with response defined as a ≥50% decrease in MADRS total score from baseline to week 3. This was assessed at day 1 and at weeks 1, 6, 9, and 12 of the study.
(b) proportion of subjects who have a sustained response at week 12. Sustained response is defined as the proportion of patients fulfilling response criteria at any visit up to and including week 3, that also fulfils response criteria at all subsequent visits up to and including week 12. Response is defined as ≥50% decrease in MADRS total score from baseline.

Another objective was to evaluate the safety and tolerability of psilocybin in subjects with TRD based on AEs, changes in vital signs, and suicidal ideation/behaviour (measured using the Columbia-Suicide Severity Rating Scale [C-SSRS]) score at all visits.

Other objectives were to evaluate the effects of psilocybin on quality of life and wellbeing, functioning and associated disability, cognitive function, and anxiety compared to 1 mg psilocybin on:
(a) Quality of life in subject EuroQoL (EQ)-5 dimension-3 level scale (EQ-5D-3L) score change from Baseline to week 3. This will also be assessed at week 12. This assessment was not mandatory.
(b) Quality of life in caregiver EQ-5D-3L score change from Baseline to week 3. This was assessed at week 12.
(c) Functioning and associated disability in the Sheehan Disability Scale (SDS) score change from Baseline to week 3. This was also assessed at week 12.
(d) Cognitive function as measured by the Digit Symbol Substitution Test (DSST) score change from baseline to week 3. This was assessed at day 1 and week 12.
(e) Level of anxiety as measured using the change in Generalised Anxiety Disorder 7 item Scale (GAD-7) total score change from baseline to week 3. This was assessed at week 12.
(f) Subject determined level of depression as measured using the change in Quick Inventory of Depressive Symptomatology Self-Rated (QIDS-SR-16) total score from Baseline to week 3. This was assessed at screening, day 1, and weeks 1, 2, 6, 9, and 12.
(g) Psychosocial functioning and predictor of response durability as measured using the change in Work and Social Adjustment Scale (WSAS) from Baseline to week 3. This was assessed at week 12.
(h) To evaluate the impact of different psilocybin doses on real life functional activity estimated from passive data streams collected on a mobile app on subjects' mobile phones. The data collected from the subject's phone included: i) number of and time of phone calls/e-mails/texts; ii) gestures used (taps, swipes, other); iii) gyroscope (orientation) of the phone (the way the phone is pointing); iv) acceleration of the phone (sudden movements of the phone); v) keystroke patterns with characters redacted; location information from the GPS; and vi) the app also maintains a histogram of daily words that the subject types on their phone. These words were stripped from their context and syntax, thus preventing the content of any particular message from being deciphered.
(i) Positive and Negative Affect Schedule (PANAS), Five Dimension Altered States of Consciousness Questionnaire (5D-ASC), 2a receptor polymorphism test and Scale to Assess Therapeutic Relationship (Clinician and Patient version, STAR-C and STAR-P, respectively) was assessed for correlation with the primary and secondary outcomes as possible predictors of response.
(j) The study endpoints are listed in Table 3B below.

TABLE 3B
Study Endpoints

| Study Endpoint | Assessments |
|---|---|
| Primary | MADRS total score from baseline (day −1) to 3 weeks post-psilocybin |
| Secondary | a) The proportion of subjects with a response (defined as a ≥50% |
| | improvement in MADRS total score from Baseline) at week 3 after the |
| | psilocybin session. |
| | b) The proportion of subjects with remission (defined as a MADRS |
| | total score ≤10) at week 3 post psilocybin. |
| | c) The proportion of subjects who have a sustained response at week 12. |
| | Sustained response is defined as the proportion of patients fulfilling response |
| | criteria at any visit up to and including week 3, that also fulfils response |
| | criteria at all subsequent visits up to and including week 12. Response is |
| | defined as ≥50% decrease in MADRS total score from baseline. |
| | d) Time to event measures: restart antidepressant medication for any reason, |
| | restart medication for continuing depressive symptoms, and relapse from a |
| | previously recovered state (clinical judgement, supported by the QIDS-SR- |
| | 16). Subjects who withdraw from the study will be censored from the time to |
| | event analysis. |
| Exploratory | Change from baseline in the following: |
| | a) Subject EQ-5D-3L at Week 3; |
| | b) Caregiver EQ-5D-3L at Week 3 (this assessment is not mandatory); |
| | c) SDS at Week 3; |
| | d) DSST at Week 3; |
| | e) GAD-7 at Week 3; |
| | f) QIDS-SR-16 at Week 3 |
| | g) WSAS at Week 3; and |
| | h) Measures derived from smart phone usage via the Mindstrong app. |
| Efficacy and | MADRS, SDS, QIDS-SR-16, GAD-7, DSST, EQ-5D-3L (subject and |
| Outcome | caregiver), WSAS |

### Study Procedures by Time Point

The study timeline includes 10 visits over a period of 12 weeks to assess the effects of psilocybin on TRD. The screening assessments for each Visit are listed in Table 4A.

### Visit 1: Screening Period

The subject was screened to evaluate suitability for the study. All subjects were seen at the clinic weekly for a minimum of 3 weeks prior to baseline (Visit 2) to ensure safe discontinuation of current antidepressant therapy required by the protocol, and to conduct psychoeducation.

At the screening visit, a number of assessments shown below in Table 4A were performed and recorded. These assessments were sometimes performed over several days, but all scales should be completed on the same day. All clinician or subject-rated assessments throughout the study were captured electronically.

If the subject was deemed eligible, they could proceed to the next visit (Visit 1a). Subjects could not begin psychoeducation or tapering off their antidepressant medication until they attend the clinic for the screening visit (Visit 1a).

At subsequent screening period visits (V1a, V1b, etc.), medications taken and any changes in medications since the previous visit, and the C-SSRS will be obtained.

### Visit 2: Baseline Visit—Day −1

The baseline visit occurred 3 to 6 weeks after initial screening (Visit 1). At the baseline visit, the subject's eligibility was confirmed by reviewing the Inclusion/Exclusion criteria and updating the medical history. The baseline visit occurred the day before the anticipated psilocybin session. The following assessments performed at the baseline visit are shown in Table 4A.

If the subject continued to meet the eligibility criteria, the trained therapist reviewed the psychoeducational material and the anticipated psilocybin session with the subject. The subject was randomized into the appropriate study group, i.e., 1 mg, 10 mg, or 25 mg psilocybin. using the IWRS (Section 8.2) and returned to the study site for treatment the following day. The research team completed a final review to ensure the subject's continued eligibility. Subjects could be progressed to Visit 3 until this approval was received.

### Visit 3: Psilocybin Session—Day 0

The psilocybin session (Visit 3) occurred the day after the baseline visit (Visit 2). In exceptional circumstances the subject visited the clinic $7 d following the baseline visit (Visit 2). A preparation session with the therapist was always conducted the day before the psilocybin session, even if the psilocybin session was not conducted the day after baseline (Visit 2). If the subject was out of the ≤ 7-day window, all baseline assessments were repeated, except randomization. The assessments provided at the psilocybin session are listed in Table 4A.

### Visit 4: Post-Dosing Day 1

The day after treatment the subject returned to the study site for a safety check and to discuss their experience during the psilocybin administration session. During this visit subjects were reminded at this dosing session to remain off any antidepressant medications until after Visit 7. The assessments provided at post-dosing session are listed in Table 4A.

### Visit 5:1 Week Post-Dosing

The subject visited the clinic 1 week (7 days±1 day) following psilocybin administration. During this visit, subjects were reminded to remain off any anti-depressant medications until after Visit 7. The assessments obtained during Visit 5 are shown in Table 4A.

### Visit 6:2 Weeks Post-Dosing

The subjects visited the clinic 2 weeks (14 days±1 day) following psilocybin administration. During this visit, subjects were reminded to remain off any anti-depressant medications until after Visit 7. The assessments obtained during Visit 6 are shown in Table 4A.

### Visit 7:3 Weeks Post-Dosing

The subjects visited the clinic 3 weeks (21 days±1 day) following psilocybin administration. During this visit, subjects were reminded to remain off any anti-depressant medications until after Visit 7. The assessments obtained during Visit 7 are shown in Table 4A.

### Visit 8:6 Weeks Post-Dosing

The subject was contacted by telephone by site staff or visit the clinic at the investigator's discretion 6 weeks (42 days±3 days) following psilocybin administration. All clinician or subject-rated assessments shown in Table 4A will be captured electronically.

### Visit 9:9 Weeks Post-Dosing

The subject was contacted by telephone 9 weeks (63 days±3 days) following psilocybin administration. The assessments obtained 9 weeks post-dosing are shown in Table 4A.

### Visit 10:12 Weeks Post-Dosing (End of Study)

The subjects visited the clinic 12 weeks (84 days±7 days) following psilocybin administration for the end of study visit. This visit was completed if the subject discontinued from the study early (early termination [ET]). The assessments obtained during Visit 10 are shown in Table 4A.

### Description of Study Procedures

Efficacy, safety, and other types of assessments performed during the study are described in Tables 3C-3E below. All measures below were captured electronically.

TABLE 3C
Efficacy Assessments

| | |
|---|---|
| Montgomery-Åsberg | MADRS evaluations will be performed by an independent |
| Depression Rating Scale | remote rater who is blinded to the subject's treatment assignment at |
| (MADRS) | baseline, day 1, and weeks 1, 3, 6, 9, and 12. The MADRS is a |
| | clinician-rated scale measuring depression severity, consisting of 10 |
| | items, each scored from 0 (normal) to 6 (severe), for a total possible |
| | score of 60; higher scores denote greater severity. The structure of the |
| | telephone-based interview will be controlled through the use of The |
| | Structured Interview Guide for the MADRS (SIGMA), which provides |
| | structured probes to ensure standardization of administration and |
| | comprehensive coverage of 10 questions. |
| Quick Inventory of | The 16-item QIDS-SR-16 a self-rated scale designed to assess |
| Depressive | the severity of depressive symptoms in the nine diagnostic symptom |
| Symptomatology | domains of a major depressive episodes, exclusive of atypical or |
| | melancholic symptoms. The QIDS-SR-16 is sensitive to change with |
| | various treatments, demonstrating its utility in research settings. The |
| | total score ranges from 0 to 27 with 0 representing no depression and |
| | 27 representing severe depression. The total score is the sum of the 9 |
| | symptom domains. The QIDS-SR-16 will be collected at every clinic |
| | visit or contact with the subject. |
| Sheehan Disability Scale | The SDS is a brief, 5-item self-report inventory that assesses |
| | functional impairment in work/school, social life, and family life. The |
| | total score ranges from 0 to 30 with 0 representing no impairment and |
| | 30 representing severe impairment. The last two items of the scale |
| | (Days Lost and Days Unproductive) do not count toward the total score. |
| | Each domain is rated on a 10-point visual analogue scale (VAS). The |
| | SDS will be obtained at baseline, and weeks 3 and 12. |
| Generalized Anxiety | The GAD-7 is useful in primary care and mental health settings |
| Disorder Scale | as a screening tool and symptom severity measure for the seven most |
| | common anxiety disorders. 32 Subjects choose one of 4 severity |
| | scores associated problems related to the common anxiety disorders |
| | and then indicate the degree to which these problems caused |
| | functional and/or social difficulties. Scores are determined by |
| | calculating the values for each column. A total score is obtained by the |
| | sum of all total column values. The GAD-7 will be obtained using the |
| | ePRO device at Baseline, and weeks 3 and 12. |
| Digit Symbol | The DSST is a neuropsychological test sensitive to brain |
| Substitution Test | damage, dementia, age and depression. The DSST consists of digit- |
| | symbol pairs followed by a list of digits. The subject will select the |
| | corresponding symbol as fast as possible. The number of correct |
| | symbols within 90 seconds will be recorded at baseline, day 1, and |
| | weeks 3 and 12. |
| EuroQoL-5 Dimension-3 | The 3-level EQ-5D version (EQ-5D-3L) was introduced by the |
| Level Scale | EuroQoL Group in 1990. The EQ-5D-3L essentially consists of 2 pages: |
| | the EQ-5D-3L descriptive system and the EQ visual analogue scale |
| | (EQ VAS). |
| | The descriptive system comprises five dimensions: mobility, |
| | self-care, usual activities, pain/discomfort and anxiety/depression. Each |
| | dimension has 3 levels: no problems, slight problems, moderate |
| | problems, severe problems and extreme problems. The subject is |
| | asked to indicate his/her health state by ticking the box next to the most |
| | appropriate statement in each of the five dimensions. This decision |
| | results in a 1-digit number that expresses the level selected for that |
| | dimension. The digits for the five dimensions can be combined into a |
| | 5-digit number that describes the subject's health state. |
| | The EQ VAS records the subject's self-rated health on a |
| | vertical VAS, where the endpoints are labelled ‘The best imaginable |
| | health state’ and ‘The worst imaginable health state’. The VAS can be |
| | used as a quantitative measure of health outcome that reflect the |
| | subject's own judgement. |
| | The EQ-5D-3L will be obtained at baseline, and weeks 3 and 12. |
| | Administration to a caregiver is not mandatory. |
| Work and Social | The WSAS is a 5-item self-report scale used to assess |
| Adjustment Scale | psychosocial functioning and to predict durability of response to |
| | antidepressant treatment. Each of the 5 questions is rated on a scale |
| | from 0 to 8, where 0 is no impairment and 8 is very severe impairment. |
| | A WSAS score above 20 appears to suggest moderately severe or |
| | worse psychopathology. Scores between 10 and 20 are associated with |
| | significant functional impairment but less severe clinical |
| | symptomatology. Scores below 10 appear to be associated with |
| | subclinical populations. |
| | The WSAS will be performed at baseline and weeks 3 and 12, |
| | and will be captured electronically. |

TABLE 3D
Safety Assessments

| | |
|---|---|
| Columbia-Suicide | The C-SSRS will be used to assess suicide potential or tendency as a |
| Severity Rating | study entry criteria and monitored throughout the study. |
| Scale (C-SSRS) | The C-SSRS is a semi-structured interview designed to assess the |
| | severity and intensity of suicidal ideation, suicidal behavior, and nonsuicidal |
| | self-injurious behavior over a specified time period. The measurement of |
| | suicidal ideation is based on 5 “yes” or “no” questions with accompanying |
| | descriptions arranged in order of increasing severity. If the patient answers |
| | “yes” to either questions 1 or 2, the intensity of ideation is assessed in 5 |
| | additional questions related to frequency, duration, controllability, deterrents, |
| | and reasons for the most severe suicidal ideation. Suicidal behavior is |
| | assessed by asking questions categorizing behaviors into actual, aborted, and |
| | interrupted attempts; preparatory behavior; and non-suicidal self-injurious |
| | behavior. |
| | If any item(s) on the C-SSRS are answered “yes”, the primary |
| | investigator or physician investigator must review the patient's responses in |
| | order to (a) at screening and Baseline determine the patient's study eligibility |
| | and potential need for referral to a mental health professional, and (b) during |
| | the study evaluate the patient's need for appropriate medical management such |
| | as a referral to a mental health professional. |
| | A significant risk of suicide is defined as a “yes” in answer to (a) |
| | questions 4 or 5 on the suicidal ideation section; or (b) any questions on any |
| | item in the suicidal behavior section. This must be reported as an SAE and |
| | followed up accordingly. Additionally, if a patient responds “yes” to any of the |
| | suicidal ideation questions 1 through 3, the investigator should apply clinical |
| | judgment to determine the need for reporting this as an AE or SAE and the |
| | need for any appropriate referral. |
| Vital Signs | Blood pressure will be measured supine, after at least 5 min at rest. |
| | The three measurements should be recorded 1 to 2 min apart, and the results |
| | averaged to inform eligibility. Respiratory rate, body temperature, and pulse |
| | rate will be obtained at screening, baseline, day 0, and day 1. |
| Electrocardiogram | Standard 12-lead ECGs will be obtained at screening (Visit 1) and day |
| | 1 (Visit 4). |
| Clinical Laboratory | Blood samples will be obtained at screening (Visit 1), day 1 (Visit 4), and week |
| Tests | 3 (Visit 7) for the following: |
| | a) Hematology: hemoglobin, hematocrit, red blood cell count, mean |
| | corpuscular hemoglobin, mean corpuscular volume, mean corpuscular |
| | hemoglobin concentration, white blood cell count (with differential), and |
| | platelet count. |
| | b) Chemistry: albumin, alkaline phosphatase, alanine aminotransferase |
| | (ALT), amylase, aspartate aminotransferase (AST), bicarbonate, |
| | bilirubin (direct, indirect, and total), calcium, chloride, creatine kinase, |
| | creatinine, gammaGT, glucose, lactate dehydrogenase, lipase, |
| | magnesium, phosphate, potassium, protein-total, sodium, urea (blood |
| | urea nitrogen), and uric acid. |
| | c) Biomarker: C-reactive protein. |
| | d) Other: 2a polymorphism at screening (Visit 1) only (optional). |
| | Urine samples will be obtained at screening (Visit 1) and baseline (Visit 2) for |
| | the following: |
| | a) Urinalysis: A dipstick urinalysis will be performed for blood, glucose, |
| | ketone, protein, pH, specific gravity, nitrite, leukocytes, bilirubin, and |
| | urobilinogen. |
| | b) Urine Drug Screen: for illicit drugs or drugs of abuse at screening |
| | (Visit 1) and baseline (Visit 2). Results of a positive drug screen will be |
| | reviewed by the study clinician for pattern of use. |
| | c) Urine Pregnancy Test: a dipstick test in women of childbearing |
| | potential at screening (Visit 1) and baseline (Visit 2). |
| Adverse Events | All AEs occurring after the subject signs the ICF and up to the last |
| | study event will be recorded. Any AEs occurring before the start of treatment |
| | (i.e., before the dose of the IP on day 0 [Visit 3]) will be recorded in the medical |
| | history. Any AE ongoing at Visit 10 (EOS/ET) will be followed until resolution |
| | or no longer considered clinically significant by the investigator. |

TABLE 3E
Other Assessment Instruments

| | |
|---|---|
| Hamilton Depression | The HAM-D 17-item scale is used to measure the degree of symptom |
| Rating Scale (HAM-D) | severity in depressed patients. The HAM-D-17 rating will be performed |
| | by the investigator using the eCOA device at the screening (Visit 1) and |
| | baseline (Visit 2) only. The total score from this assessment will be used |
| | as eligibility criteria prior to treatment (minimal total symptom score ≥ |
| | 18). The Structured Interview Guide for the HAM-D-17 (SIGH-D) will be |
| | administered. This will be captured electronically. |
| Mini International | The MINI was designed as a brief structured interview for the |
| Neuropsychiatric | major Axis I psychiatric disorders in DSM-5 and International |
| Interview (MINI) | Classification of Diseases-10. Validation and reliability studies have |
| | been done comparing the MINI to the Structured Clinical Interview for |
| | DMS-5 Patient Edition and the Composite International Diagnostic |
| | Interview (a structured interview developed by the World Health |
| | Organization). Version 7.0.2 of the MINI will be used for this study. The |
| | results of these studies show that the MINI has similar reliability and |
| | validity properties, but can be administered in a much shorter period |
| | (mean 18.7 ± 11.6 min, median 15 min) than the above referenced |
| | instruments. It can be used by clinicians after a brief training session. |
| | At screening (Visit 1), subjects will be assessed for MDD, as |
| | documented by DSM-5 criteria, and the lack of other psychiatric |
| | diagnoses will be confirmed by use of the MINI. |
| Massachusetts General | The MGH-ATRQ is a self-rated scale used to determine |
| Hospital-Antidepressant | treatment resistance in major depressive disorder. The scale examines |
| Treatment History | the efficacy (improvement from 0%, not improved at all to 100% |
| Questionnaire (MGH- | completely improved, and adequacy of a treatment. Subjects are asked |
| ATRQ) | by clinician about treatment adherence to each medication trial and |
| | examines the subjects' antidepressant history to identify pseudo- |
| | resistance and treatment resistance. The MGH-ATRQ will be collected |
| | at screening (Visit 1) only. This will be captured electronically. |
| McLean Screening | The MSI-BPD is a commonly used measure to assess for BPD. |
| Instrument for Borderline | The scale consists of 10 items based on the DSM-5 BPD criteria; the |
| Personality Disorder | first 8 items represent the first eight criteria in the DSM-5 for BPD |
| | diagnosis, while the last two questions assess the paranoia and |
| | dissociation criteria for BPD. Scores for the MSI-BPD range from 0 to |
| | 10, with each item rated as “1” if present and “0” if absent. A score of 7 |
| | or higher indicates a likelihood for the subject to meet criteria for BPD. |
| | The MSI-BPD will be collected at screening (Visit 1) only. This will be |
| | captured electronically. |
| Adult Self-Report Scale | The ASRS is a self-reported questionnaire used to determine the |
| | presence of attention-deficit/hyperactivity disorder in adults. The |
| | screener (6-item) version of the scale will be used in this study. The first |
| | question concerns inattention and the other 5 questions assess |
| | hyperactivity-impulsivity. Each question is answered on a 5-point Likert- |
| | type scale, ranging from “Never” to “Very Often”. This scale will be |
| | obtained at Screening (Visit 1) only and will be captured electronically. |
| Scale to Assess | The STAR-P and STAR-C is a 12-item measure assessing the |
| Therapeutic | therapeutic relationship between patient and clinician on three |
| Relationships - Patient | components: Collaboration, Positive Clinician Input, and Emotional |
| and Clinician Version | Difficulties (clinician version)/Non-Supportive Clinician (patient version) |
| | input. The Collaboration subtest reflects a good rapport and a shared |
| | understanding of goals, mutual understanding, openness, and trust. |
| | Positive Clinician Input reflects the perception (by the subject) of the |
| | clinician to encourage, support, and listen to the subject. Emotional |
| | Difficulties/Non-Supportive Clinician Input reflect problems in the |
| | relationship. The range of total scores for both versions is 0-48, with a |
| | higher score suggesting better therapeutic relationships. Each version of |
| | the scale takes approximately 5 min to complete. Total scores and |
| | subscale totals can be obtained. The STAR-P and STAR-C will be |
| | collected at Baseline (Visit 2) only. These will be captured electronically. |
| Five Dimension Altered | The 5D-ASC measures the acute drug effects using 5 primary |
| States of Consciousness | dimensions and respective subdimensions to assess alterations in |
| Questionnaire | mood, perception, and experience of self in relation to environment and |
| | thought disorder. The 5 dimensions include oceanic boundlessness, |
| | anxious ego dissolution, visionary restructuralization, auditory |
| | alterations, and reduction of vigilance. This will be administered |
| | immediately after the psilocybin session on Day 0 (Visit 3). This will be |
| | captured electronically. |
| The Positive and | The PANAS measures the acute emotional drug effects, and |
| Negative Affect Schedule | comprises 2 mood scales that measure positive and negative affect. |
| | Subjects respond to 20 items using a 5-point scale that ranges from |
| | “slightly or not at all (1)” to “extremely (5)”. A total higher score on the |
| | positive affect questions indicates more of a positive affect while a lower |
| | score on the negative affect questions indicates less of a negative effect. |
| | This will be administered at baseline (Visit 2), the day after the psilocybin |
| | session (Visit 4), and at week 3 (Visit 7). This will be captured |
| | electronically. |
| Emotional Breakthrough | The EBI is a VAS that describes the intensity and quality of the |
| Inventory | emotional experience following the psilocybin session, developed by the |
| | Imperial College London. This is collected on day 1 (Visit 4) |
| | electronically. |
| Mindstrong Application | If the subject opts to participate in the Mindstrong portion of the study, |
| | an app will be activated on the subject's mobile phone at Visit 1; it will be |
| | deactivated at the EOS. |
| | Mobile data collection: The Mindstrong app will collect data from the |
| | subject's smart phone and periodically send these data back to the |
| | Mindstrong secure database. The subject will not need to do anything to |
| | collect these, except use their phone as they normally do. The data |
| | collected from the subject's phone will include: |
| | a) Number of and time of phone calls/e-mails/texts (content will not be |
| | collected); |
| | b) Location information from the GPS; |
| | c) Orientation of the smart phone (the way the phone is pointing); |
| | d) Acceleration of the smart phone (sudden movements of the phone); |
| | e) Keystroke information with characters redacted; and |
| | f) Gestures used (taps, swipes, other). |
| | The app may also maintain a list of target words and the subject's use- |
| | frequency of these words. These words will be completely stripped from |
| | their context, thus preventing the content of any particular message from |
| | being deciphered. |
| | The data collection application does NOT collect information on who or |
| | what number the subject calls or receive calls from. The data collection |
| | application does NOT capture the content of messages the subject |
| | sends or receives in a readable format. No personally identifiable |
| | information is captured and all other data are encrypted on the |
| | smartphone before transmission to the Mindstrong data enclave hosted |
| | on Amazon Web Service's cloud computers. |
| | Specifically, the application does NOT collect: |
| | a) Email addresses of the emails that the subject sends or receives; |
| | b) Phone numbers of the phone calls that the subject makes or receives; |
| | c) Phone numbers of the text messages that the subject makes or |
| | receives; and |
| | d) Messages sent or received in a readable format. |

### Subject Recruitment

A total of 233 subjects were enrolled in the study examining pelocybin treatment for treatment resistant depression.

Subjects meeting all the following inclusion criteria as shown in Table 4F below were considered for admission into the study. No deviations were permitted from the inclusion criteria.

TABLE 3F
Study Inclusion Criteria

| No. | Criteria |
|---|---|
| 1 | Signed consent form |
| 2 | 18 years of age or older at screening; (USA only) less |
| | than age 55 at screening. |
| 3 | At least moderate MDD (single or recurrent episode as |
| | informed by DSM-5; if single episode, duration of ≥3 |
| | months and ≤2 years) based on medical records, clinical |
| | assessment and documented completion of the version |
| | 7.0.2 MINI |
| 4 | HAM-D-17 (17-item) score ≥18 at screening (Visit 1) |
| | and at Baseline (Visit 2) |
| 5 | Failure to respond to an adequate dose and duration of |
| | 2, 3, or 4 pharmacological treatment for the current |
| | episode as determined through the MGH-ATRQ and |
| | using the supplementary advice on additional |
| | antidepressants not included in MGH-ATRQ. |
| | Augmentation with an add on treatment counts as a |
| | second treatment, provided it is approved for the |
| | adjunctive treatment of MDD in that country. |
| 6 | McLean Screening Instrument for Borderline Personality |
| | Disorder <7 at screening (Visit 1) |
| 7 | Have successfully discontinued all antidepressant |
| | medications at least 2 weeks prior to Baseline (Visit 2) |
| 8 | Ability to complete all protocol required assessment tools |
| | without any assistance or alteration to the copyrighted |
| | assessments, and to comply with all study visits |

Subjects meeting any of the psychiatric or general medical exclusion criteria shown in Table 4G and Table 4H below were not be enrolled in the study. No deviations were permitted from the exclusion criteria.

TABLE 3G
Psychiatric Exclusion Criteria

| No. | Exclusion Criteria |
|---|---|
| 1 | Current or past history of schizophrenia, psychotic |
| | disorder (unless substance induced or due to a medical |
| | condition), bipolar disorder, delusional disorder, paranoid |
| | personality disorder, schizoaffective disorder, or borderline |
| | personality disorder, or any serious psychiatric |
| | comorbidity as assessed by medical history and structured |
| | clinical interview (version 7.0.2 MINI). |
| 2 | Prior electroconvulsive therapy and/or ketamine for |
| | current episode |
| 3 | Current cognitive behavioral therapy (CBT) that will not |
| | remain stable for the duration of the study. CBT cannot be |
| | initiated within 21 days of Baseline. |
| 4 | Current (within the last year) alcohol or substance abuse |
| | as informed by DSM-5 at screening (Visit 1) |
| 5 | Significant suicide risk as defined by (1) suicidal ideation |
| | as endorsed on items 4 or 5 on the C-SSRS within the |
| | past year, at screening or at baseline, or; (2) suicidal |
| | behaviors within the past year, or; (3) clinical assessment |
| | of significant suicidal risk during subject interview |
| 6 | Depression secondary to other medical conditions. |
| 7 | Other personal circumstances and behavior judged to be |
| | incompatible with establishment of rapport or safe |
| | exposure to psilocybin, including exposure to psilocybin |
| | within the past year and use of psychedelics, such as |
| | ayahuasca, during the current depressive episode. |

TABLE 3H
General Medical Exclusion Criteria

| No. | Exclusion Criteria |
|---|---|
| 1 | Women who are pregnant, nursing, or planning a |
| | pregnancy. Subjects who are sexually active must agree |
| | to use a highly effective contraceptive method throughout |
| | their participation in the study. Women of child bearing |
| | potential must have a negative urine pregnancy test at |
| | screening (Visit 1) and baseline (Visit 2) |
| 2 | Cardiovascular conditions: recent stroke (<1 year from |
| | signing of ICF), recent myocardial infarction (<1 year from |
| | signing of ICF), hypertension (blood pressure >140/90 |
| | mmHg or [USA only] QTc >450 msec) or clinically |
| | significant arrhythmia within 1 year of signing the ICF |
| 3 | Uncontrolled insulin-dependent diabetes. |
| 4 | Seizure disorder. |
| 5 | Positive urine drug screen for illicit drugs or drugs of |
| | abuse (USA only: to include but not limited to opiates, |
| | PCP, cocaine, amphetamines, methamphetamines, |
| | benzodiazepines, barbiturates and methadone; cannabis |
| | for medicinal purposes or recreational use is permitted) at |
| | Visit 1 and Visit 2. Any positive urine drug test will be |
| | reviewed with subjects to determine the pattern of use and |
| | eligibility will be determined at the investigator's discretion |
| | in conjunction with the MM. |
| 6 | Current enrolment in any investigational drug or device |
| | study or participation in such within 30 days of screening |
| | (Visit 1) |
| 7 | Current enrolment in an interventional study for |
| | depression or participation in such within 30 days of |
| | screening (Visit 1) |
| 8 | Abnormal and clinically significant results on the physical |
| | examination, vital signs, ECG, or laboratory tests at |
| | screening (Visit 1) |
| 9 | Any other clinically significant cardiovascular, pulmonary, |
| | gastrointestinal, hepatic, renal or any other major |
| | concurrent illness that, in the opinion of the investigator, |
| | may interfere with the interpretation of the study results or |
| | constitute a health risk for the subject if he/she takes part |
| | in the study |

All subjects were seen weekly for at least 3 weeks prior to the psilocybin session (Visit 3) to ensure the safe discontinuation of current antidepressant therapy required by the protocol. Rescreening of subjects considered not eligible for the study was allowed.

### Psilocybin Dose and Administration

Subjects were randomized to the appropriate therapy after it was confirmed at baseline (Visit 2) that the subject remained eligible to participate in the study. Randomization was stratified by country. Each subject was assigned 1 treatment bottle containing 5 capsules packaged in a double-blind fashion, depending on the randomized treatment arm, the bottle will contain 1 of the following:
(a) 10-mg treatment bottle: 2×5-mg capsules and 3×placebo capsules;
(b) 25-mg treatment bottle: 5×5-mg capsules; or
(c) 1-mg treatment bottle: 1×1-mg capsule and 4×placebo capsules.

After a light breakfast taken 2 h prior to dosing and under observation of research personnel, the 5-capsule dose was swallowed with a full glass of water. Due to the number of capsules in a dose, additional water may be necessary to swallow the dose. Research personnel ensured the entire 5-capsule dose was swallowed.

To prepare for the drug experience, the subject took the appropriate dose of psilocybin and lied down on a couch in a room with dim lights and a standard playlist of relaxing music playing quietly. A trained therapist was present with the subject at all times.

The effects of psilocybin usually start about 20 to 30 min after administration, becoming most intense in the first 90 to 120 min, and gradually subside in 5 to 6 h. The subjects were asked to remain in the room for the duration of the session regardless of the intensity of the effects, preferably lying down and mostly silent unless they had a concern or need to communicate a discomfort or seek reassurance from the therapist, or use the restroom. The therapist checked-in′ with the subject (i.e., ask how the subject is doing) in 30- to 60-min intervals post-dosing. A light meal and fruit was available for the subject.

About 5 to 6 h after dosing, trained therapist discussed the psilocybin experience with the subject. The subject was discharged 6 to 8 h post-dosing when, in the opinion of the investigator, the acute effects of psilocybin were resolved. The subject was accompanied home. The site was notified that they have returned home safely, and in the absence of receiving a phone call site staff to directly contact the subject.

### Psychotherapeutic Goals of Psilocybin:

The psychotherapeutic goals of the psilocybin session were to:
(a) Ensure psychological safety essential for optimal clinical efficacy;
(b) Allow subject's subjective experience to unfold naturally within the boundaries of the therapeutic intention set at the preparation;
(c) Maintain subject's attention and awareness on the experience of the present moment thus allowing exposure and processing of the challenging emotional states and personal memories; and
(d) Generation of insights and solutions for the resolution of challenging personal situations, conflicts and traumatic experiences.

### Methods:

Psychotherapeutic methods of the psilocybin session had the following objectives:
(a) Psychological safety: effective management of anxiety is essential to safety, tolerability and efficacy of psilocybin;
(b) It has also been shown in previous studies that severe prolonged anxiety in the beginning of the experience could adversely affect the efficacy of psilocybin, therefore the management of anxiety during the onset of the session is an essential skill of psilocybin therapists. Anxiety during the onset of action is not uncommon, and the therapists are specially trained to recognize and actively manage subjects through such periods of anxiety until the subject is comfortable enough to continue on their own. The examples of such active guiding include:
(i) Remember you enrolled in this research study of psilocybin for treatment of your depression. As psilocybin takes effect, some anxiety and fear are expected. It is part of the process. Remember we practice relaxation and breathing experiences for situations like this?
(ii) Let's take deep breaths together and focus on the sensations of the breath throughout the body. As you do this, pay attention to the rhythm of your breath and watch it becoming deeper and slower. Let go of muscle tension with every exhalation.

Therapists are asked to validate the feeling of anxiety without providing interpretations of perceptual disturbances or guiding subjects towards a particular image or memory, other than encouraging them to stay relaxed and open to the emergent experiences.

In the modern research setting, any anxiety during the onset of psilocybin action responded well to reassurance and meditation.

In preparation for the psilocybin session, therapists demonstrated and practiced skills of self-directed inquiry and experiential processing with subjects. Subjects were encouraged to face and explore their experience, including the challenging ones. During the peak and later stages of the session, self-directed inquiry and experiential processing become essential for subjects to develop a different perspective on their personal challenges and conflicts, and to generate their own solutions. Such self-generated insights are not only therapeutic because of the emotional resolution, but also empowering to subjects. This approach is used in MDMA-assisted psychotherapy for treatment of PTSD and is particularly helpful in the event of emergent traumatic memories.

### Structure of the Psilocybin Session:

The psilocybin session is supported by two appropriately qualified research personnel. The study psychiatrist were in the immediate vicinity of the session to respond to any emergencies.

On the day of the session, subjects arrived early in the morning with the goal to take the psilocybin treatment around 9 am.

Prior to dose administration, a team of psychotherapists reviewed the rules and structure of the session with the subject again. Once all the questions were answered, and the subject reconfirmed their consent for the session, they were administered the psilocybin (5 capsules) with a full glass of water. Delaying the intake of psilocybin would induce unnecessary anxiety in subjects, therefore it is recommended to have the treatment room prepared for the start of the session before subject's arrival.

The treatment rooms in all trial sites are furnished in soft furniture in muted colors to create a non-clinical calming feel. All treatment rooms are equipped with a high-resolution sound system that allows for simultaneous ambient and earphone listening.

Subjects were then encouraged to lie down, practice relaxation and breathing exercises, and listen to calming music. Therapists might want to revisit the intention for the treatment session with the subject and again, ask the question “What would it look and feel like to be free of depression?” Such revisions immediately prior to the session provide an implicit direction for the subjective experience during the psilocybin session.

Once the effects of psilocybin become noticeable, subjects were encouraged to put on Mindfold eyeshades and earphones and focus on their internal experience.

Psychotherapists sat at knee level of the subject on both sides of the couch. Psychotherapists were discouraged from reading, using laptops or phones, eating or drinking other than water during the first 2-3 hours of the session.

If adequately prepared, subjects tolerated the onset well using the skills practiced during preparation period. Psychotherapists offer support in the form of reminders, encouragement, grounding hand holding, or active guiding, should the challenging experiences arise. The best ways for support, and boundaries of physical touch were discussed and practiced during the preparation. In general, therapists were instructed to provide therapeutic grounding above shoulder level only. In case of subjects with a history of physical and sexual abuse, therapeutic touch were limited to hand and forearm areas only, or to the form of physical support that was agreed to during preparation.

Therapists were trained in the skill of recognizing when to allow the subject's experience to unfold naturally. During the peak experience, especially in the case of non-dual or full ego-dissolution experience, subjects are usually silent and may appear comfortable, even blissful. In such cases, no active guiding is needed.

As the drug effects start to subside, subjects again might become engaged with emergent narratives.

In case of prolonged anxiety or distress, therapists could choose to actively guide subjects through such experiences without interpreting or judging the experiences or giving advice. Once subjects were comfortable, they were encouraged again to engage in introspection.

At the end of the session and after the effects of psilocybin are no longer evident, subjects become more talkative and interactive. The role of the therapists now was to ensure that experiential processing was complete with some emotional resolution. In those cases where there was still anxiety or despair at the end of the session, subjects were encouraged to relax and reflect for a longer period of time. The provisions were made for therapists to stay with the subject until the effects of the drug fully subsided, and subject was assessed to be comfortable and fully sober. This was assessed through engaging in ‘small talk’ about non-contentious topics unrelated to the content of the session. The therapist might for example ask:
(a) It is almost 6 o'clock. What do you normally do at this time of the day?
(b) What do you usually have for dinner?
(c) What do you like to cook?

Subjects and therapists were discouraged to discuss the content of the session until the next day to avoid premature consolidation of the insights.

At the end of the session, therapists and subjects could have a light meal together to mark the closure of the session. The subject's family or friends could also be invited to join the meal if the subject consents.

After the safety assessments, subjects were discharged in the care of a family member or a friend. Please refer to the section of safety for more detailed information on discharge assessments and unexpected adverse events.

### Before the Session:

On the day of the psilocybin session, the subject arrived at the clinical center between 8 am and 9 am. It is essential for the therapist and the supportive assistant (the therapeutic team) to prepare the room and take care of all the logistics prior to the subject's arrival.

The therapist and assistant should welcome the subject shortly following his/her arrival and allow for the expression of any questions or concerns. Since subjects are likely to be at least mildly anxious, it is important to validate their anxiety and assure them it is common to be anxious prior to a new experience. The time following arrival and prior to entering the treatment room should be as minimal as possible, as “waiting outside” (even if reading a book) tends to increase anxiety.

The behavioral rules are reviewed again. The subject should reconfirm that he/she:
(a) Will stay in the room for the duration of the session;
(b) Will follow the therapist's instructions as all directions are given entirely to ensure their safety;
(c) Have an accurate mutual understanding of ways the therapist can provide support during the session, including interpersonal grounding, guided imagery and breathing exercises;

Key components of mental set/intention are also reviewed with the subject and include the following:
(a) Every experience is welcome; nothing to censor or avoid;
(b) Face anything that looks potentially frightening as rapidly and openly as possible;
(c) Reach out for grounding, support or sharing at any time;
(d) Remember key instructions: Trust, Let Go, Be Open;
(e) Remember to breathe deeply if needed;
(f) You'll never be left alone;
(g) No need to entertain the therapist/assistant; and
(h) This is your day. We're with you, whether you need us or not.

Once all the agreements are reconfirmed and the subject is settled in the treatment room, the study investigator or designee offers 5 capsules of the psilocybin with a full glass of water. After the subject takes the capsules and drinks all the water, he/she should settle back on the couch, listen to the music, focus on his/her breathing and relax. This is often when the subject may share photographs or meaningful objects he/she has brought, or when he/she may leaf through an art book-often with the therapist and assistant sitting on both sides of the subject on the couch. As the initial effects of psilocybin are beginning or about to begin, a final trip to the bathroom is offered before the subject reclines and accepts the eyeshade and headphones.

Before the drug's effects begin, it is helpful to re-establish the subject's stated goals for the treatment and to revisit the question: “What does feeling better or recovery feel like?” The subject is reminded that their primary task during this session is to simply collect new and interesting experiences which can then be discussed with the therapist during the integration phase. The therapist can remind the subject of the purpose of the psilocybin therapy and the role of experiential processing, namely allowing the subject to be open and curious to whatever arises and encountering thoughts and feelings previously unknown to them. It should be emphasized that this process inherently requires letting go and a willing passivity to the psychedelic experience; the willingness to let go is correlated with better outcomes in psilocybin therapy. The therapist should remind the subject that the therapeutic team will be supporting them at all times.

Subjects are encouraged to relax and focus on internal experience, but are allowed to move around, sit up, talk, and stretch as needed. Occasionally subjects may feel the need to move around or express their emotions physically. All expressions, including physical expressions are encouraged.

### Onset of Action:

### Setting and Music

A standardized playlist is employed in all sessions regardless of the apparent level of psilocybin dosage. It begins with soft background music before the subject enters the room and continues through the various phases of a typical high-dose session. It may include some periods of silence. In intense sessions, the choice of music rarely appears to influence experiential content, but it can provide strong nonverbal support and engagement with unfolding inner content unique to each subject. In the latter two hours of a psilocybin session, most any music can be appreciated and explored. The subject is instructed to accept and explore the music as the day progresses, irrespective of their usual personal preferences or current emotional responses. Criticizing and trying to control the music has often been found to be a symptom of resistance to unfolding content. Therapists may choose to deviate from the playlist in highly unusual situations but allowing the standardized playlist to unfold generally proves effective and frees the therapist to focus on the subject. The playlist is skillfully designed to provide variety in a context of accumulated experience with many different persons undergoing psychedelic therapy.

### Managing Anxiety

Transient anxiety is often reported as subjects encounter changing psychological content. Such anxiety might be viewed as natural and even necessary. It can manifest in different ways, ranging from mild intractability and avoidance of the emerging experiences to extreme paranoia. In most cases, anxiety resolves on its own accord and can be minimized with skillful interpersonal support. Psilocybin provides a unique opportunity for a subject to normalize anxiety and view it as excitement and experience the encounter with honest ambivalence.

During the acute onset of action, the subject might experience perceptual changes in visual, auditory or olfactory modes, and a range of unusual physical sensations. These experiences could be anxiety-provoking, particularly in psychedelically naïve subjects. During preparation, the therapist encourages the subject to become curious about these experiences and to freely explore them.

If the subject continues to manifest anxiety and emotional distress, the therapist may offer therapeutic touch or interpersonal grounding, if that is something the subject has agreed to during preparation and has been rehearsed. An example of interpersonal grounding would be: “I want to state again my commitment to be here for you. I will do whatever is necessary to make this a safe place for you so that you can fully experience whatever comes up. If what comes up is difficult, I'd like you to try and stay with it and explore it as much as you can. Please ask me for whatever you need.”

If the subject is agitated and/or frightened, simple reminders could be helpful such as “You remember that you are participating in a clinical trial of a new medication for your depression. During preparation, we talked about possible anxiety, unusual sensations and intense emotions. This is simply the drug taking effect. It is safe; you will not be harmed. These challenging experiences pass by very quickly if you relax and just watch them. You will return to everyday reality as the effect of psilocybin wanes.”

The therapist encourages the subject to focus inwards and fully immerse him/herself in all aspects of the experience. The subject may want to practice guided imagery or breathing relaxation techniques in preparation.

### Managing Distractions and Avoidance

Occasionally, the subject will try to avoid emerging experiences or distract him/herself while trying to regain cognitive control over the unusual state of their mind. The therapist must recognize that such distractions could take different forms. The subject might want to engage in a conversation or prematurely describe in detail their experience, visions or insights. When this occurs, the therapist and assistant aim to remain as silent as possible, thereby enabling the subject and his/her inner experience to direct the course of the psilocybin session. Active listening skills may be required if the subject engages the therapist in conversation; this should be paired with prompts to encourage the subject to continue focusing attention on present experiences. As shown in the example below, the therapist does not add any new information or even words, but still acknowledges the subject.
Subject: “Yeah, everything has this shiny quality to it, like it's made of this otherworldly metal.”
Therapist: “I wonder if you could stay with that otherworldly experience and see where it takes you.”

And if the subject continues to engage in the conversation, the therapist can say: “Perhaps you can focus attention to the details so that we can talk about it later. If you talk about this now, you will be less able to focus on the experience and might miss important details.”

Here the therapist again acknowledges the subject's comment without introducing new topics. Then the therapist guides the subject back to his/her present experience.

Sometimes a subject might ask to go to the bathroom or have a drink of water. The sudden and urgent character of such requests might suggest that they are really trying to avoid emerging material. In such cases, the therapist might suggest: “We will take a bathroom break at the end of this piece of music” or “I will get you water in a little while. Why don't you put the eye shades back on and relax for a few minutes?”

If a subject is trying to avoid a difficult experience, they might listen to the suggestion and relax.

### Challenging Shifts Versus Adverse Events

Emotions and feelings can shift quickly during psilocybin experiences. Such shifts must be differentiated from distress that requires active guidance. It is essential that the therapist use his/her clinical judgment to establish whether the subject is in need of the therapist's quiet and non-intrusive presence, or active guidance. During preparation, the subject has been informed that they can ask for support from the therapist for any reason at any time, especially when they do not feel able to navigate through a particular experiential sequence.

Support can be in the form of therapeutic touch, verbal reassurance, guided imagery or a breathing exercise. It is advisable to apply one technique at a time to allow for minimal intervention and interference with the subject's unique process. In the preparatory session, the therapist and subject may have discussed the most helpful ways to support in case of emotional distress. It might be useful to remind the subject about this conversation by stating: “You remember I told you that anxiety is to be expected and we have agreed that I may support you though physical grounding. If you feel anxious, please reach out for my hand or let me know what else might help you.” Another example of how to handle emotional distress in the subject is shown below:
Subject: “The negative feelings are getting really overwhelming now”
Therapist: “Do you remember the conversation we had about negative experiences?”
Subject: “Yeah. You suggested I should sit with them.”
Therapist: “How does that sound to you?”
Subject: “It's going to be difficult, but I'll try. If I can't, can you help me?”
Therapist: “Yes I'll do all I can to help you. I'd like you to tune out and just stay focused on what you're feeling right now.”
Subject: “Okay, I'm doing that. It doesn't feel good.”
Therapist: “As you focus on it, are you feeling it altering or changing in any way?”
Subject: “It feels like it's coming and going.”
Therapist: “How do you feel about that?”
Subject: “It's difficult.”
Therapist: “What's making it difficult?”
Subject: “I'm not sure.”
Therapist: “Would it be helpful if I held your arm?”
Subject: “Yes, I think that would help.”

As soon as the subject is comfortable, the therapist should encourage him/her to return to further exploration. Remind the subject that you will be there if needed and that there will be a chance during integration to talk through the experience.

Therapists may talk with clinicians/therapists and may coordinate psychotherapy outside the trial. Information should be provided regarding the integration of the psilocybin session with any psychotherapy the subject is currently receiving (and will continue to receive throughout the duration of the study and cannot have been initiated within 21 days of baseline).

### Use of Rescue Medication:

If, in the judgement of the investigator, the subject cannot be calmed through all the available methods and is presenting a current threat to him/herself or others, then the use of benzodiazepines and/or antipsychotics is allowed. These should be administered according to their respective approved prescribing information and dose levels. The subject should be made safe in the least invasive and distressing way possible.

### Peak Experience:

With adequate dosage and an acute onset of action, a ‘peak’ experience may occur about 60-90 minutes after ingestion. Factors that influence the quality and intensity of peak experiences include the subject's ability to stay with whatever arises in awareness, their ability to relax and let go of expectations and fears, dose of psilocybin, etc.

Non-dual ego-dissolution experiences have been shown to positively correlate with the magnitude and durability of the clinical response, so this state needs to be attended with care. The goal of the therapist is to encourage and support the subject in being fully present and relaxed in this state. Verbal communication should be minimal.

Subjects who reach peak non-dual experience tend to be quiet; even non-responsive. In the unlikely event that a subject would express a need to prematurely attempt to share such an experience, it should be viewed as distraction or avoidance. The therapist needs to encourage the subject to stay quiet and focus on the sensations, insights and feelings, and to collect as many details about the experience as possible so as to be able to relate the story later. However, in true ego-loss and non-dual experiences, there is no ego present. When recalled in memory, subjects usually claim such states to be beyond language.

Transcendent non-dual experiences are frequent, especially during high-dose psilocybin sessions. There may be perceptions that extend well beyond the usual sense of self, such as feelings of oneness in which the subject experiences an openness and enhanced connection to their own humanity and to the surrounding environment. Such experiences can be difficult to interpret and may challenge a therapist's own worldview. The therapist is not required to understand, support or even have an opinion about the nature or content of these experiences, but it is essential that they validate them and convey openness toward the subject's own view of them without dismissing or pathologizing any experience based on its unusual content. These experiences may provide the subject with a perspective that goes beyond identification with their personal narrative.

It is equally important that a therapist not show disappointment when a subject does not report a profound transcendental experience. The therapist should remain mindful of his/her own reactions and responses to the subject's experiences and validate any and all of them. However, that doesn't mean that the therapist must agree with unusual, magical thinking. Validation of the experiences simply means acknowledging the courage of opening up to the experience and the possibility that any experience will serve the intention of the session.

### Conclusion of Psilocybin Session:

The drug effects last for 4-6 hours. During the final phase, it's important that the subject doesn't prematurely terminate the session by excessive talking. Even if little appears to be happening, periodic returns to the couch with music, headphones and eyeshade often provide for not only unexpected new experiential content, but safe and complete closure. The use of music is continued and light conversations between the therapist and subject are encouraged. This serves the dual role of re-orienting the subject to everyday reality and enabling the therapist to assess for immediate risks. By having a light casual conversation about dinner or the weather, the therapist assesses whether the subject is struggling to re-enter reality, or is distressed by the content of the session.

This can be discussed over a light meal with the subject, which—if they choose—can include the subject's family members. The therapist needs to be mindful that the subject is adequately reoriented to everyday reality, and be certain that the subject is fully competent to travel before making the journey home. If the subject requires a longer period of time to return to normality, the therapist is expected to remain with the subject. If the subject requires emotional support due to a difficult session, the therapist is required to support the subject until the discomfort is resolved and the subject is fully back in everyday reality. The therapist should use open questions and empathic attention, as in previous sessions, to ensure that the subject feels supported.

Specific Criteria for Discharge from the Facility on the Day of Drug Administration:

Subjects will remain in the treatment facility for a full 8 hours after the start of the session, to ensure that the psilocybin effects have fully subsided. Subjects are then assessed for safety by the therapists and the study clinician. Blood pressure will be monitored before discharge.

Subjects are observed in the facility to ensure that they are fully ambulatory, have good balance, are psychologically stable, and can perform activities of daily living. Subjects are also asked to complete the QIDS-SR-16 (Quick Inventory of Depressive Symptomatology-Self-Rated), C-SSRS (Columbia-Suicide Severity Rating Scale) and 5D-ASC (5 Dimensions of Altered States of Consciousness). These scales will be performed by the study team and will allow the clinician to assess subject's emotional and cognitive stability, risk for suicidality and whether the perception-altering effects of psilocybin have subsided.

When judged safe and functional, subjects are discharged in the care of a friend or a family member, referred to as a companion. This person is not the aforementioned caregiver who completes the EQ-5D-3L. Plans for the follow-up visit the next morning are confirmed prior to discharge.

Subjects who live more than 30 minutes away from the treatment center may be offered nearby accommodation in a hotel.

When the subject is ready to leave, he/she will be given information regarding the time and location of the next session (safety assessment and integration within 24 hours of the psilocybin session) as well as contact numbers if help is needed before then. The therapist is expected to be available 9 am-5 pm on weekdays and the emergency services number can be used on weekends and evenings. The subject must agree not to drive or use alcohol during the evening following the psilocybin session. The therapist should check in with the subject and family member(s) later in the evening to confirm that the subject is comfortable and safe.

Subjects who may have an unusually prolonged experience or remain in severe distress and are considered at risk for adverse reactions will be asked to stay for observation overnight and will be supported by research personnel.

In case of psychotic reaction, subjects will be assessed by a study psychiatrist and hospitalized as needed. The emergency protocol specifies oral benzodiazepines, followed by antipsychotics as needed.

### Concomitant Therapy:

All prescription and non-prescription medications (e.g., over-the-counter drugs and herbal supplements) that subjects report taking during the 30 days prior to screening (Visit 1) was assessed and recorded at that visit. For each medication, documentation listed the trade or generic name, the total daily dose including units (or the dose, units and scheduled and actual frequency of administration if the medication is not taken daily), the route of administration, and the reason for use.

Concomitant medication refers to all drugs and therapies used from the time the informed consent form was signed through the end of study participation.

Changes, additions, or discontinuations to medications was assessed and recorded during each study visit. All as-needed prescriptions were converted to reflect actual number of pills or dose taken per day.

Medications for the management of concurrent anxiety and insomnia, or non-psychiatric medications that have a potential psychotropic effect were permitted within the following limitations. From the initial screening (Visit 1) through final study visit (Visit 10, EOS), subjects were permitted to use benzodiazepines (up to 2 mg of lorazepam equivalents per day for insomnia and anxiety if it is not taken within 12 h before the psilocybin dose. Prescription and nonprescription medications with psychoactive properties that were used as needed for nonpsychiatric conditions (e.g., pseudoephedrine for allergies or cold symptoms; zopiclone for sleep disorders) were not used no more than 2 times a week and not in the 12 h before any study assessment. Documentation of the use of adjunctive anxiolytics, hypnotics, or medication with potential psychotropic properties (including over-the-counter preparations) were obtained at each clinic visit.

Therapy considered necessary for the subject's welfare may be given at the discretion of the study clinician.

In some embodiments, the following plan was followed to manage the occurrence of physiological adverse events during the psilocybin session:
(a) Headaches-Tylenol 650 mg taken by mouth, once a day;
(b) Symptomatic elevated blood pressure-short-acting beta blocker on the formulary once a day by mouth or IV;
(c) Chest pain-ECG, nitroglycerin sublingual, acetylsalicylic acid once a day and further evaluation as needed;
(d) Nausea, Vomiting, Aspiration-precautions, ice chips.

Rescue medications could be used during and after the psilocybin session. The decision to medicate a subject depended on whether the monitors and responsible physician judge that they were capable of maintaining the safety of the patient and others without medical intervention.

Benzodiazepine anxiolytics is the pharmacological intervention of choice in case of acute psychological distress (e.g., medications such as lorazepam or alprazolam that have a rapid onset, a short time until peak plasma concentration, and a short duration of therapeutic action; the oral route is preferable because IV injection procedures may further exacerbate the subject's anxiety).

Antipsychotic medications (e.g., risperidone) were available in the event that an adverse reaction escalates to unmanageable psychosis.

In case of development of acute anxiety or psychotic symptoms requiring pharmacological intervention, the subject was managed appropriately. Rescue medication was administered according to their respective approved prescribing information and dose levels. The subject was discharged from the clinic when, in the opinion of investigator, the condition had stabilized. The subject was accompanied home. The site was notified by the subject that they had returned home safely, and in the absence of receiving a phone call site staff directly contacted the subject.

### Adverse Events

Throughout the course of the study, all adverse events (AEs) were monitored and recorded and included the AE's description, start and end date, seriousness, severity, action taken, and relationship to the treatment. If AEs occurred, the first concern was the safety of the study subjects.

An AE is any untoward medical occurrence in a subject administered a pharmaceutical product and which does not necessarily have to have a causal relationship with this treatment. An AE can therefore be any unfavorable and unintended sign (including an abnormal laboratory finding, for example), symptom, or disease temporally associated with the use of a medicinal product, whether considered related to the medicinal product.

Medical interventions such as surgeries, diagnostic procedures, and therapeutic procedures are not AEs but the action taken to treat the medical condition. They should be recorded as treatment of the AEs.

### Efficacy and Outcome Measures:

Efficacy and outcome measures include MADRS, QIDS-SR-16, SDS, GAD-7, DSST, EQ-5D-3L (subject and caregiver), and WSAS.

The change from baseline in MADRS total score at week 3 will be evaluated with a mixed effects model for repeated measures analysis. The model includes treatment, visit, study site, prior psychedelic experience, treatment by visit interaction, subject as a random effect, and baseline MADRS total score. Comparison of the psilocybin optimal dose versus 1 mg psilocybin was performed at the 0.05 testing level. A sensitivity analysis was performed on the primary mixed model repeated measures model adding treatment by study site or country interaction into the model. If it was significant (at the 10% level), then further investigations of sites were performed.

Four additional efficacy endpoints that are dichotomous variables (proportion of subjects who are responders, remitters, and sustained responders) were analyzed using the Cochran Mantel Haenszel chi square test, stratified by country, to compare the psilocybin optimal therapeutic dose versus 1 mg psilocybin. A stepdown procedure to correct for multiplicity was employed.

Time-to-event measures will be evaluated using Kaplan-Meier methods.

Response and remission rates were summarized at each visit.

Change from baseline in continuous efficacy measures, including the QIDS-SR-16 scale and GAD-7 total scores at each point, were analyzed based on last observation carried forward data using an analysis of covariance model, with treatment and study site as factors, and the respective baseline score as the covariate. The exploratory analyses for quality of life and wellbeing, functioning and associated disability, cognitive function, and anxiety are not hierarchical; there was no correction for multiplicity in these analyses.

Scores for all efficacy endpoints, including dimension scores of the EQ-5D-3L and the EQ VAS, were summarized over time using descriptive statistics for all visits during the observation period.

The covariate selection process was addressed in the SAP to be approved before any analyses were undertaken.

Continuous behavior sampling was tested with the Mindstrong application technology in a subgroup of smart phone users consented to this part of the study. Subjects who did not consent to the app installation, or don't have a smart phone, were not excluded from the study. Following the installation of the Mindstrong app on the subject's smart phone, the app begins to collect data from the smart phone and periodically send these data back to the secure database. The subject did not need to do anything at this stage, except use their phone as they normally do. As explained above, personal data from mobile phone usage was analyzed in a sub-group of consenting subjects.

The correlation between sensor data, keyboard behavior, or voice and speech metrics was assessed for correlation with standard clinical assessments and the ability of app features to identify impending relapse before clinical change is apparent with traditional ratings. The goal of these exploratory assessments is to identify mobile use patterns (app features) predictive of clinically significant mood changes that will have an impact on care and treatment outcomes.

### Analysis of Safety:

Safety analyses were performed using data from the safety population. Safety was evaluated based of AEs, vital signs, clinical laboratory assessments, and ECG findings.

### Columbia-Suicide Severity Rating Scale (C-SSRS)

Item scores from the C-SSRS, all visits by randomized treatment, the item scores from the version assessing suicidality since the last visit, and all postbaseline visits (Visit 3 to Visit 10, inclusive) by treatment will be tabulated. Summary statistics of suicidal ideation and suicidal behavior following psilocybin administration was presented by randomized treatment.

### Adverse Events

AEs were coded by Preferred Term (PT) using the Medical Dictionary for Regulatory Activities (MedDRA) classification. All reported AEs with onset or worsening after the administration of study medication were included in the analysis. The incidence of AEs was summarized by treatment group, and by severity and relationship to psilocybin. Serious AEs and AEs leading to withdrawal from the study will be tabulated.

A TEAE is defined as any AE that has an onset on or after the dose of psilocybin, or any pre-existing condition that has worsened on or after the dose of psilocybin.

The incidence of TEAEs and treatment-related AEs were summarized by maximum severity and most-related relationship to psilocybin by MedDRA primary system organ class and PT. The summary included the total number and percentage of subjects reporting an event. In counting the number of events reported, a continuous event, i.e., reported more than once and which did not cease, will be counted only once; non continuous AEs reported several times by the same subject were counted as multiple events.

### Electrocardiographic Data

The ECG data was summarized descriptively based on measures of change in each ECG parameter from Visit 1 to post-treatment (Visit 4). Frequency tabulations of the abnormalities were provided. ECG variables analyzed included heart rate, PR interval, QRS interval, QT interval, and corrected QT interval using the following correction methods: QT corrected according to Bazett's formula and QT corrected according to Fridericia's formula.

### Laboratory Data

Laboratory data (hematology and blood chemistry parameters) was presented for each treatment group using descriptive statistics, including mean and mean change from baseline values at each scheduled time point. Shift tables displayed numbers of subjects with normal/abnormal values at Baseline versus post-treatment. The frequency of laboratory abnormalities was tabulated. By-subject data listings flagged laboratory values that were outside normal reference ranges or markedly abnormal findings.

### Vital Signs

Changes from baseline in vital signs, blood pressure (systolic and diastolic), body temperature, pulse rate, and respiratory rate, were summarized for each treatment group using descriptive statistics. The last measurement obtained prior to treatment administration served as baseline.

The percentage of subjects with values outside clinically important limits were summarized. A listing of weight and height at Visit 1 will be provided.

### Demographic and Baseline Characteristics

Treatment groups were compared with respect to subject demographics and baseline characteristics will be summarized using descriptive statistics, no formal statistical analysis tests were performed.

### Results:

In the randomised, controlled, double-blind trial, a single dose of investigational COMP360 psilocybin was given to 233 patients in conjunction with psychological support from specially trained therapists. All patients discontinued antidepressants prior to participation. The trial was powered to compare two active doses of COMP360, 25 mg and 10 mg, against a comparator 1 mg dose. The MADRS was assessed by independent raters who were remote from the trial site, and blind to intervention and study design, effectively creating a triple blind.

FIG. 9B shows the patient disposition from the clinical trial.

### Efficacy

Table 4 shows the rate of participants starting new treatments in the 3 dosing groups. The data show a similar rate of participants starting new treatments; however, the 25 mg typically started new treatments at a later timepoint than those in the 10 mg and 1 mg arms.

TABLE 4
Rate of Participants Starting New Treatments

| | COMP360 | | COMP360 | | COMP360 | | | |
|---|---|---|---|---|---|---|---|---|
| Participants started | 25 mg | | 10 mg | | 1 mg | | Overall | |
| new treatment for | N = 79 | | N = 75 | | N = 79 | | N = 233 | |
| depression | n (%) | | | | | | | |
| Day 2 | 0 | | 0 | | 0 | | 0 | |
| Week 1 | 0 | | 5 | (6.7) | 4 | (5.1) | 9 | (3.9) |
| Week 3 | 4 | (5.1) | 4 | (5.3) | 9 | (11.4) | 17 | (7.3) |
| Week 6 | 11 | (13.9) | 10 | (13.3) | 9 | (11.4) | 30 | (12.9) |
| Week 9 | 10 | (12.7) | 5 | (6.7) | 3 | (3.8) | 18 | (7.7) |
| Week 12 | 5 | (6.3) | 3 | (4.0) | 5 | (6.3) | 13 | (5.6) |
| Total | 30 | (38.0) | 27 | (36.0) | 30 | (38.0) | 87 | (37.3) |

FIG. 9C and FIG. 9D show the change from baseline in MADRS total score (Primary analysis: main analysis)—MMRM analysis. There was a fast onset of action with treatment differences between 25 mg vs 1 mg apparent from day 2 and maintained to week 6. Statistically significant primary endpoint at week 3, 25 mg vs 1 mg was observed.

FIG. 9E shows the number of MADRS responders by visit (Responder: ≥50% decrease in MADRS total score from baseline) using generalised linear mixed model (GLMM) analysis. The 25 mg group demonstrated rapid response with treatment differences from day 2 to week 3 compared to 1 mg group.). The 25 mg group vs the 1 mg group showed a −6.6 difference on the MADRS depression scale at week 3 (p<0.001). The 25 mg group demonstrated statistical significance on the MADRS efficacy endpoint on the day after the COMP360 psilocybin administration (p=0.002). The 10 mg vs 1 mg dose did not show a statistically significant difference at week 3 (36.7% (29 patients) in 25 mg group showed response at week 3, compared with 17.7% (14 patients) in 1 mg group). The results show that a single 25 mg dose of COMP360 demonstrated a highly statistically significant and clinically relevant reduction in depressive symptom severity after three weeks, with a rapid and durable treatment response. Table 5 shows the number of MADRS responders at Day 2, Week 1 and Week 3.

TABLE 5
Number of MADRS Responders at Day 2, Week 1, and Week 3

| | | | |
|---|---|---|---|
| | COMP360 | COMP360 | COMP360 |
| | 25 mg | 10 mg | 1 mg |
| Visit/Statistic | N = 79 | N = 75 | N = 79 |

Day 2

| | | | |
|---|---|---|---|
| LS mean | −15.0 | −12.0 | −9.0 |
| (95% CI) | (−17.6, −12.3) | (−14.8, −9.3) | (−11.7, −6.4) |
| LS mean diff vs 1 mg | −5.9 | −3.0 | — |
| (95% CI) | (−9.6, −2.3) | (−6.7, 0.7) | |
| p-value | 0.001 | 0.116 | — |

Week 1

| | | | |
|---|---|---|---|
| LS mean | −13.1 | −9.1 | −6.8 |
| (95% CI) | (−15.6, −10.6) | (−11.7, −6.4) | (−9.4, −4.3) |
| LS mean diff vs 1 mg | −6.3 | −2.2 | — |
| (95% CI) | (−9.8, −2.8) | (−5.8, 1.3) | |
| p-value | <0.001 | 0.214 | — |

Week 3

| | | | |
|---|---|---|---|
| LS mean | −12.0 | −7.9 | −5.5 |
| (95% CI) | (−14.6, −9.4) | (−10.6, −5.2) | (−8.2, −2.8) |
| LS mean diff vs 1 mg | −6.6 | −2.4 | — |
| (95% CI) | (−10.1, −2.8) | (−6.1, 1.2) | |
| p-value | <0.001 | 0.193 | — |

N = number of participants in the population;
n = number observed;
LS = least squares;
CI = confidence interval

Table 6 shows the number of MADRS responders at Week 6, Week 9 and Week 12.

TABLE 6
Number of MADRS Responders at Week 6, Week 9, and Week 12

| | | | |
|---|---|---|---|
| | COMP360 | COMP360 | COMP360 |
| | 25 mg | 10 mg | 1 mg |
| Visit/Statistic | N = 79 | N = 75 | N = 79 |

Week 6

| | | | |
|---|---|---|---|
| LS mean | −11.7 | −6.0 | −6.3 |
| (95% CI) | (−14.6, −8.8) | (−9.1, −3.0) | (−9.3, −3.4) |
| LS mean diff vs 1 mg | −5.3 | 0.3 | — |
| (95% CI) | (−9.4, −1.3) | (−3.8, 4.4) | |
| p-value | 0.010 | 0.891 | — |

Week 9

| | | | |
|---|---|---|---|
| LS mean | −9.4 | −6.1 | −5.4 |
| (95% CI) | (−12.5, −6.3) | (−9.4, −2.9) | (−8.6, −2.2) |
| LS mean diff vs 1 mg | −4.0 | −0.7 | — |
| (95% CI) | (−8.3, 0.3) | (−5.1, 3.6) | |
| p-value | 0.070 | 0.743 | — |

Week 12

| | | | |
|---|---|---|---|
| LS mean | −9.6 | −5.7 | −6.0 |
| (95% CI) | (−12.8, −6.3) | (−9.1, −2.3) | (−9.3, −2.7) |
| LS mean diff vs 1 mg | −3.6 | 0.3 | — |
| (95% CI) | (−8.0, 0.9) | (−4.2, 4.8) | |
| p-value | 0.116 | 0.900 | — |

N = number of participants in the population;
n = number observed;
LS = least squares;
CI = confidence interval

FIG. 9F shows the number of MADRS remitters by visit (Remitter: MADRS total score≤10) using generalised linear mixed model (GLMM) analysis. The 25 mg group demonstrated rapid remission with treatment differences from day 2 to week 3 compared to 1 mg group. 29.1% (23 patients) in 25 mg group were in remission at week 3, compared with 7.6% (6 patients) in 1 mg group. At least twice the number of patients in the 25 mg group showed response and remission at week 3 and week 12, compared with the 1 mg group.

FIG. 9F shows the number of MADRS sustained responders at week 12 using logistic regression analysis. The 25 mg group had a higher proportion of sustained responders compared to 1 mg group. The protocol-defined sustained response (protocol-defined sustained response=patients meeting the MADRS response criteria from week 3 until week 12) up to week 12 was double, with 20.3% of patients in the 25 mg group vs 10.1% in the 1 mg group. Using a definition of sustained response that is consistent with other TRD studies (sustained response=patients meeting the MADRS response criteria at week 3 and at week 12, and at least at one visit out of week 6 and week 9), the difference was more than double, with 24.1% of patients in the 25 mg group vs 10.1% in the 1 mg group.

Analyses of exploratory measures including anxiety, self-reported depression, positive and negative affect, and functioning, showed greater improvements for patients receiving a 25 mg dose of COMP360 psilocybin compared with those receiving a 1 mg dose after three weeks. These exploratory measures also showed that patients in the 25 mg dose group of COMP360 psilocybin therapy had advantages over those in the 1 mg group. On the PANAS scale measuring positive and negative affect, patients in the 25 mg group had a higher increase in positive affect (eg including feeling interested, excited, strong) and a greater decrease in negative affect (eg including feeling distressed, upset, afraid) on the day after COMP360 administration and at the questionnaire's final administration at week 3. On scales measuring anxiety (GAD-7), self-rated depression (QIDS-SR-16) and functioning (SDS and WSAS), a greater improvement was also shown at week 3, by patients in the 25 mg group compared with the 1 mg group.

Affect: results from the PANAS (Positive Affect and Negative Affect Schedule) showed higher positive affect and lower negative affect changes from baseline in the 25 mg vs 1 mg groups at day 2 and week 3. For positive affect and negative affect, there was a least squares mean (95% confidence intervals) treatment difference favouring the 25 mg vs 1 mg group at week 3:6.17 (3.53, 8.82) and −3.18 (−5.59, −0.77), respectively.

Anxiety: changes from baseline in the GAD-7 (Generalised Anxiety Disorder-7 item scale) total score were greater in the 25 mg group vs the 1 mg group at week 3. For GAD-7, a least squares mean (95% confidence intervals) treatment difference favouring the 25 mg vs 1 mg group was found at week 3: −1.79 (−3.35, −0.23).

Self-reported depression: changes from baseline in the QIDS-SR-16 (Quick Inventory of Depressive Symptomatology-Self Rated-16 item scale) total score were greater in the 25 mg group at weeks 1, 2 and 3, compared with the 1 mg group. The least squares mean treatment difference (95% confidence intervals) at week 3 was −2.78 (−4.62, −0.95).

Functioning: changes from baseline in the SDS (Sheehan Disability Scale) and the WSAS (Work and Social Adjustment Scale) were greater in the 25 mg group vs the 1 mg group at week 3. There was a treatment difference of −6.49 (95% confidence interval=−9.52, −3.46) on the SDS 25 mg vs 1 mg at week 3 and a treatment difference of −5.11 (95% confidence interval=−8.39, −1.82) on the WSAS between the 25 mg vs 1 mg at week 3.

Quality of life: no differences were seen between the groups on the EQ-5D-3L (EuroQol-5-Dimension-3-Level Scale) total score and EuroQoL-Visual Analogue Scale-all groups showed an improvement over time. On the EQ-5D-3L total score, the 25 mg vs 1 mg least squares mean treatment differences at week 3 was 0.06 (95% confidence intervals=−0.03, 0.15). On the EuroQoL Visual Analogue Scale, the 25 mg vs 1 mg treatment difference at week 3 was 6.77 (95% confidence interval=−0.37, 13.90). An analysis of sustained responders in the 25 mg group found that changes in quality of life were clinically meaningful, with mean scores in these patients returning to “normal” levels and maintained to 12 weeks.

Cognition: no differences were seen between the groups on the DSST (Digit Symbol Substitution Test)—all groups showed an improvement over time. The least squares mean treatment difference between the 25 mg vs 1 mg groups at week 3 was 1.32 (95% confidence intervals=−1.00, 3.64).

An analysis of sustained responders in the 25 mg group found that changes in quality of life, self-reported depression severity, and functioning were clinically meaningful, with mean scores for these patients returning to “normal” levels and maintained to 12 weeks, the end of the trial.

Durability is being studied in a one-year follow-up study.

### Safety:

COMP360 was generally well tolerated, with more than 90% of treatment-emergent adverse events (TEAEs) being mild or moderate in severity. In the study, 179 participants reported at least one treatment emergent adverse event (TEAE): 66 participants (83.5%) in the 25 mg group; 56 participants (74.7%) in the 10 mg group, and 57 participants (72.2%) in the 1 mg group.

Table 7 shows the treatment emergent adverse events by group. The AE incidence is higher in the 25 mg group overall; however, key mood-related AEs (euphoric mood, depression, depressed mood, suicidal ideation) do not have a higher incidence in the 25 mg arm.

TABLE 7
Treatment Emergent Adverse Events by Group

| | COMP360 | COMP360 | COMP360 | |
|---|---|---|---|---|
| MedDRA TEAE | 25 mg | 10 mg | 1 mg | Overall |
| Preferred Term | N = 79 | N = 75 | N = 79 | N = 233 |
| (at least 5%) | n (%) | | | |
| Headache | 27 (34.2) | 16 (21.3) | 20 (25.3) | 63 (27.0) |
| Nausea | 18 (22.8) | 7 (9.3) | 4 (5.1) | 29 (12.4) |
| Fatigue | 12 (15.2) | 5 (6.7) | 7 (8.9) | 24 (10.3) |
| Insomnia | 8 (10.1) | 11 (14.7) | 14 (17.7) | 33 (14.2) |
| Anxiety | 7 (8.9) | 13 (17.3) | 3 (3.8) | 23 (9.9) |
| Mood altered | 7 (8.9) | 3 (4.0) | 1 (1.3) | 11 (4.7) |
| Back pain | 6 (7.6) | 0 | 3 (3.8) | 9 (3.9) |
| Dizziness | 6 (7.6) | 1 (1.3) | 1 (1.3) | 8 (3.4) |
| Suicidal ideation | 5 (6.3) | 5 (6.7) | 4 (5.1) | 14 (6.0) |
| Myalgia | 5 (6.3) | 2 (2.7) | 1 (1.3) | 8 (3.4) |
| Euphoric mood | 4 (5.1) | 5 (6.7) | 4 (5.1) | 13 (5.6) |
| Depression | 4 (5.1) | 6 (8.0) | 5 (6.3) | 15 (6.4) |
| Abdominal pain upper | 4 (5.1) | 2 (2.7) | 1 (1.3) | 7 (3.0) |
| Irritability | 4 (5.1) | 2 (2.7) | 1 (1.3) | 7 (3.0) |
| Panic reaction | 4 (5.1) | 1 (1.3) | 1 (1.3) | 6 (2.6) |
| Depressed mood | 3 (3.8) | 5 (6.7) | 4 (5.1) | 12 (5.2) |
| Paraesthesia | 3 (3.8) | 4 (5.3) | 1 (1.3) | 8 (3.4) |
| Thinking abnormal | 0 | 4 (5.3) | 0 | 4 (1.7) |

MedDRA = Medical Dictionary for Regulatory Activities;
TEAE = treatment emergent adverse event;
N = number of participants in the population;
n = number observed
In the study, 12 participants reported at least one treatment emergent serious adverse event (TESAE): 5 participants (6.3%) in the 25 mg group; 6 participants (8.0%) in the 10 mg group, and 1 participants (1.3%) in the 1 mg group.

Table 8 shows the treatment emergent serious adverse events by group. The TESAE incidence was comparable between 25 mg and 10 mg groups and lower in the 1 mg arm.

TABLE 8
Treatment Emergent Serious Adverse Events

| | COMP360 | COMP360 | COMP360 | |
|---|---|---|---|---|
| MedDRA | 25 mg | 10 mg | 1 mg | Overall |
| TESAE | N = 79 | N = 75 | N = 79 | N = 233 |
| Preferred Term | n (%) | | | |
| Any TESAE | 5 (6.3) | 6 (8.0) | 1 (1.3) | 12 (5.2) |
| Suicidal behaviour | 3 (3.8) | 0 | 0 | 3 (1.3) |
| Intentional self-injury | 2 (2.5) | 2 (2.7) | 1 (1.3) | 5 (2.1) |
| Suicidal ideation | 2 (2.5) | 2 (2.7) | 0 | 4 (1.7) |
| Drug withdrawal | 1 (1.3) | 0 | 0 | 1 (0.4) |
| syndrome* | | | | |
| Adjustment disorder | 1 (1.3) | 0 | 0 | 1 (0.4) |
| with anxiety | | | | |
| Adjustment disorder | 1 (1.3) | 0 | 0 | 1 (0.4) |
| with mixed | | | | |
| anxiety and depressed | | | | |
| mood | | | | |
| Depression | 0 | 1 (1.3) | 0 | 1 (0.4) |
| Hospitalisation | 0 | 1 (1.3) | 0 | 1 (0.4) |

MedDRA = Medical Dictionary for Regulatory Activities;
TEAE = treatment emergent adverse event;
N = number of participants in the population;
n = number observed

Overall, 209 patients completed the study; there were five withdrawals from the 25 mg group, nine from the 10 mg, and 10 from the 1 mg.

The majority of treatment-emergent adverse events (TEAEs) occurring on the day of COMP360 administration resolved on the same day or the day after (77.4%); most of these events were mild or moderate in nature, eg headache, nausea, fatigue. All TEAEs involving hallucination (which only occurred in the 25 mg and 10 mg groups) and illusion (which occurred in all groups) started on the day of administration and resolved on the same day.

TEAEs of suicidal ideation, suicidal behaviour and intentional self-injury were seen in all groups, as is regularly observed in a TRD population. Two thirds of the patients had previous thoughts of wishing to be dead, as assessed by a suicidality scale completed during patient screening; this included all patients reporting one of these adverse events.
There was no difference between the three groups post-administration in scores from item 10 on the MADRS, which measures suicidality and was assessed by a blinded remote rater; mean scores across treatment groups were lower than baseline at all subsequent time points.
27 of the TEAEs of suicidal ideation, suicidal behaviour and intentional self-injury occurred across 17 patients, with seven patients in the 25 mg group, six in the 10 mg group, and four in the 1 mg group
14 of these events were reported as treatment-emergent serious adverse events (TESAEs); these occurred across nine patients, with four patients in the 25 mg group, four patients in the 10 mg group, and one in the 1 mg group.
The majority of these TESAEs (10 events out of 14) occurred at least one week after the COMP360 psilocybin session

All suicidal behaviours occurred at least one month after the psilocybin therapy session and all patients reporting these events were non responders at the time of event onsets.

### Example 3. Long Term Follow-Up Study of Example 2

### Study Design

The following example is a long term follow up of study of a sustained responder population from Example 2. 58 participants from Example 2 were selected for a long term 52 week follow up study. Of the 58 patients, 22 received a 25 mg dose of psilocybin, 19 received a 10 mg dose of psilocybin, and 17 received a 1 mg dose of psilocybin. The sustained responder population was characterized as participants from Example 2 that had a ≥50% reduction in their baseline MADRS total score at weeks 3 and 12 and at least one of week 6 and week 9. None of the patients in this study received treatment for depression before their week 12 MADRS visit in Example 2. Patients were analyzed for a change in MADRS total score from Baseline to 12, 16, 20, 24, 28, 40, and 52 weeks.

### Study Objectives and Results

The primary endpoint of this study was to determine the median time to a depressive event after administration of psilocybin. The depressive event includes:
Initiation of new antidepressant treatment (first new antidepressant treatment in time period only);
Hospitalization due to depression/suicidality;
Suicide attempt, prevention of an imminent suicide attempt, or completed suicide;
Increased suicidality measured by worsening on MADRS item 10;
Active suicidal ideation measured by the C-SSRS;
MADRS worsening; and
Discontinuation for adverse event or lack of efficacy.

The median time to a depressive event was longer for the majority of participants in the 25 mg group compared to both the 10 mg and 1 mg groups (Table 9). As shown in Table 9, the median time to a depressive event for patients administered 25 mg of psilocybin was 189 days (24 days, 95% CI) whereas the median time to a depressive event for patients administered 10 mg and 1 mg of psilocybin was 43 days and 21 days, respectively.

TABLE 9
Estimate (95% CI) of KM quartiles for time to any depressive event (days)

| Quartile (KM) | 25 mg (N = 22) | | 10 mg (N = 19) | | 1 mg (N = 17) | |
|---|---|---|---|---|---|---|
| Q1 | 24.0 (2.0, 132.0) | days | 19.0 (2.0, 41.0) | days | 6.0 (2.0, 14.0) | days |
| Q2 (median) | 189.0 (24.0, —) | days | 43.0 (19.0, 142.0) | days | 21.0 (3.0, 78.0) | days |
| Q3 | — (199.0, —) | days | — (43.0, —) | days | 78.0 (21.0, —) | days |

The secondary endpoint of this study was to determine the change in MADRS total score from Baseline to 12, 16, 20, 24, 28, 40, and 52 weeks post treatment. From baseline to 52 weeks, none of the sustained responders in any arms started new treatments for depression. The results indicated that treatment differences favored the patients administered 25 mg vs 1 mg in terms of sustained response observed to Week 28 (Tables 10-13). Note, “n*” changed to account for subjects that dropped out the long term study. Table 10 shows MADRS for responders (participants with a ≥50% reduction from baseline in MADRS total score at a specified timepoint); Table 11 shows MADRS for remitters (participants with a MADRS total score≤10 at a specified timepoint); Table 12 shows MADRS for sustained responders (defined as the proportion of patients fulfilling response criteria at Week 3 postdosing (in the lead-in studies), that also fulfils response criteria at all subsequent visits up to and including Week 12, 16, 20, 24, 28, 40, and 52); and Table 13 shows MADRS for relaxed sustained responders (relaxed sustained population includes participants that had a ≥50% reduction in their baseline MADRS total score at weeks 3 and 12 and at least one of week 6 and week 9).

TABLE 10
MADRS for responders at Day 2 - Week 52

| | 25 mg | 10 mg | 1 mg |
|---|---|---|---|
| | (N = 22) | (N = 19) | (N = 17) |
| Visit | n/n* (%) | n/n* (%) | n/n* (%) |
| Day 2 | 12/22 (54.5) | 8/19 (42.1) | 3/17 (17.6) |
| Week 1 | 10/22 (45.5) | 7/18 (38.9) | 2/14 (14.3) |
| Week 2 | — | — | — |
| Week 3 | 9/20 (45.0) | 6/15 (40.0) | 1/11 (9.1) |
| Week 6 | 6/18 (33.3) | 3/11 (27.3) | 3/9 (33.3) |
| Week 9 | 4/16 (25.0) | 5/10 (50.0) | 1/8 (12.5) |
| Week 12 | 6/16 (37.5) | 4/10 (40.0) | 2/7 (28.6) |
| Week 16 | 7/12 (58.3) | 1/5 (20.0) | 0/5 |
| Week 20 | 6/11 (54.5) | 1/3 (33.3) | 2/4 (50.0) |
| Week 24 | 5/11 (45.4) | 2/3 (66.7) | 2/4 (50.0) |
| Week 28 | 4/9 (44.4) | 2/4 (50.0) | 1/4 (25.0) |
| Week 40 | 1/6 (16.7) | 3/5 (60.0) | 2/4 (50.0) |
| Week 52 | 2/6 (33.3) | 2/5 (40.0) | 0/4 |

Percentages are based on the number of participants in the Full Analysis Set with non-missing observations (identified as n* in the table).

TABLE 11
MADRS for remitters at Day 2 - Week 52

| | 25 mg | 10 mg | 1 mg |
|---|---|---|---|
| | (N = 22) | (N = 19) | (N = 17) |
| Visit | n/n* (%) | n/n* (%) | n/n* (%) |
| Day 2 | 9/22 (40.9) | 8/19 (42.1) | 2/17 (11.8) |
| Week 1 | 8/22 (36.4) | 4/18 (22.2) | 1/14 (7.1) |
| Week 2 | — | — | — |
| Week 3 | 8/20 (40.0) | 4/15 (26.7) | 0/11 |
| Week 6 | 5/18 (27.8) | 2/11 (18.2) | 1/9 (11.1) |
| Week 9 | 3/16 (18.8) | 5/10 (50.0) | 0/8 |
| Week 12 | 4/16 (25.0) | 3/10 (30.0) | 0/7 |
| Week 16 | 4/12 (33.3) | 0/5 | 0/5 |
| Week 20 | 5/11 (45.5) | 1/3 (33.3) | 0/4 |
| Week 24 | 4/11 (36.4) | 1/3 (33.3) | 1/4 (25.0) |
| Week 28 | 3/9 (33.3) | 2/4 (50.0) | 0/4 |
| Week 40 | 0/6 | 3/5 (60.0) | 1/4 (25.0) |
| Week 52 | 0/6 | 2/5 (40.0) | 0/4 |

Percentages are based on the number of participants in the Full Analysis Set with non-missing observations (identified as n* in the table). Participants enrolled from COMP 001 did not have a Week 2 MADRS visit.

TABLE 12
MADRS for sustained responders at Week 12 - Week 52

| | 25 mg | 10 mg | 1 mg |
|---|---|---|---|
| | (N = 22) | (N = 19) | (N = 17) |
| Visit | n/n* (%) | n/n* (%) | n/n* (%) |
| Week 12 | 4/16 (25.0) | 3/10 (30.0) | 0/7 |
| Week 16 | 4/12 (33.3) | 1/5 (20.0) | 0/5 |
| Week 20 | 4/11 (36.4) | 0/3 | 0/4 |
| Week 24 | 4/11 (36.4) | 0/3 | 0/4 |
| Week 28 | 2/9 (22.2) | 0/4 | 0/4 |
| Week 40 | 0/6 | 0/5 | 0/4 |
| Week 52 | 0/6 | 0/5 | 0/4 |

Percentages are based on the number of participants in the Full Analysis Set for which a sustained responder status could be uniquely defined based on their observed MADRS total scores and that did not take any new treatment for depression after baseline (identified with n* in the table).

TABLE 13
MADRS for relaxed sustained responders at Week 12 - Week 52

| | 25 mg | 10 mg | 1 mg |
|---|---|---|---|
| | (N = 22) | (N = 19) | (N = 17) |
| Visit | n/n* (%) | n/n* (%) | n/n* (%) |
| Week 12 | 5/16 (31.3) | 4/10 (40.0) | 0/7 |
| Week 16 | 5/12 (41.7) | 1/5 (20.0) | 0/5 |
| Week 20 | 5/11 (45.5) | 0/3 | 0/4 |
| Week 24 | 5/11 (45.5) | 1/3 (33.3) | 1/4 (25.0) |
| Week 28 | 3/9 (33.3) | 1/4 (25.0) | 0/4 |
| Week 40 | 0/6 | 1/5 (20.0) | 0/4 |
| Week 52 | 1/6 (16.7) | 1/5 (20.0) | 0/4 |

Percentages are based on the number of participants in the Full Analysis Set for which a sustained responder status could be uniquely defined based on their observed MADRS total scores and that did not take any new treatment for depression after baseline (identified with n* in the table).

### Example 4: Clinical Study Examining Psilocybin for the Treatment of Treatment-Resistant Depression in Non-Responder Patient Populations

The purpose of this study is to evaluate the effectiveness of a second dose of psilocybin in treating treatment-resistant depression in patients that did not respond to a first dose of psilocybin. In this study, patients are assigned to three groups based on the experimental dose administered to the patient. (Table 14). A MADRS will be determined for each patient at baseline (prior to administration of a first dose psilocybin) and up to week 3 after administration of the first dose. If the patient is determined to be unresponsive to psilocybin treatment after administration of a first dose psilocybin (“non-responder”), the patient is administered a second dose of psilocybin equal to the amount of the first dose (i.e., both the first and second dose of psilocybin will be 1 mg, 10 mg, or 25 mg). A clinical evaluation, such as MARDS, is performed after the first dose of psilocybin to identify the patient as a non-responder. The clinical depression evaluation may be performed on any time point from Day 1 through week 3 after the first dose of psilocybin. The second dose of psilocybin is administered about 3 weeks after the first dose of psilocybin. It is contemplated that patients who did not respond to a first dose of psilocybin will respond to a second dose psilocybin.

TABLE 14
Study Groups for Example 4

| Study Group | Dose |
|---|---|
| Group 1 | 1 mg |
| Group 2 | 10 mg |
| Group 3 | 25 mg |

All, documents, patents, patent applications, publications, product descriptions, and protocols which are cited throughout this application are incorporated herein by reference in their entireties for all purposes.

1. A method of treating treatment-resistant depression with psilocybin in a subject that did not respond to a first dose of psilocybin, comprising administering a second dose of psilocybin about 3 weeks after administering the first dose of psilocybin.

2. A method of treating treatment-resistant depression in a subject in need thereof, comprising administering a first dose of psilocybin to the subject;
measuring the subject's depressive symptoms using a clinical depression evaluation after administering the first dose of psilocybin;
identifying the subject as a non-responder to the first dose of psilocybin; and
administering a second dose of psilocybin to the subject 3 weeks after administering the first dose.

3. The method of claim 1 or 2, wherein the subject exhibits no or substantially no reduction in symptoms of depression after administering the first dose of psilocybin.

4. The method of claim 1, comprising identifying the subject as non-responsive to the first dose of psilocybin using a clinical depression rating scale.

5. The method of claim 2 or 4, wherein the clinical depression evaluation is Montgomery-Åsberg Depression Rating Scale (MADRS).

6. The method of any one of claims 2-5, wherein the subject that is non-responsive has a change in baseline in a MADRS of about-10 to 0 or less than 50% after administering the first dose of psilocybin.

7. Th method of claim 6, wherein the subject has a change in baseline in the MADRS of −10, −9, −8, −7, −6, −5, −4, −3, −2, or −1 after administering the first dose of psilocybin.

8. The method of claim 6 or 7, wherein the change in baseline in the MADRS is measured on Day 2, Day 3, Day 4, Day 5, or Day 6 after administering the first dose of psilocybin, or 1 week, 2 weeks, or 3 weeks after administering the first dose of psilocybin.

9. The method of any one of claims 1-7, wherein the subject is responsive to the second dose of psilocybin.

10. The method of any one of claims 1-9, wherein the subject exhibits a reduction in symptoms of depression after administering the second dose of psilocybin.

11. The method of any one of claims 1-10, comprising identifying the subject as responsive to the second dose of psilocybin using a clinical depression rating scale.

12. The method of claim 11, wherein the clinical depression rating scale is MADRS, and the subject has a change in baseline in the MADRS of −11 to −30 after administering the second dose of psilocybin.

13. The method of claim 12, wherein the subject has a change in baseline in the MADRS of −11, −12, −13, −14, −15, −16, −17, −18, −19, −20, −21, −22, −23, −24, or −25, after administering the second dose of psilocybin.

14. The method of claim 12 or 13, wherein the change in baseline in the MADRS is measured on Day 2, Day 3, Day 4, Day 5, or Day 6 after administering the second dose of psilocybin, or 1 week, 2 weeks, or 3 weeks after administering the second dose of psilocybin.

15. The method of any one of claims 1-14, wherein the first dose comprises 1 mg of psilocybin and second dose comprises 1 mg of psilocybin.

16. The method of any one of claims 1-14, wherein the first dose comprises 10 mg of psilocybin and second dose comprises 10 mg of psilocybin.

17. The method of any one of claims 1-14, wherein the first dose comprises 25 mg of psilocybin and second dose comprises 25 mg of psilocybin.

18. The method of any one of claims 1-17, wherein the psilocybin is administered to the subject in a pharmaceutical composition.

19. The method of claim 18, wherein the pharmaceutical composition comprises psilocybin, pregelatinized starch, and sodium stearyl fumarate.

20. The method of claim 18 or 19, wherein the pharmaceutical composition comprises about 1%-10%, by weight, psilocybin.

21. The method of any one of claims 18-20, wherein the pharmaceutical composition comprises about 85-99%, by weight, pregelatinized starch.

22. The method of any one of claims 18-21, wherein the pharmaceutical composition comprises about 0.5%-2%, by weight, sodium stearyl fumarate.

23. The method of any one of claims 1-22, wherein the psilocybin is crystalline psilocybin characterized by XRPD peaks at 11.5±0.1, 12.0±0.1, 14.5±0.1, 17.5±0.1 and 19.7±0.1°2θ.

24. The method of claim 23, wherein the crystalline psilocybin has a chemical purity of greater than 97% as determined by HPLC analysis.

25. The method of claim 24, wherein the crystalline psilocybin has no single impurity of greater than 2%.

26. A method of treating treatment-resistant depression in a subject that responded to a first dose of psilocybin, comprising administering a second dose at least about 26 weeks after administering the first dose.

27. The method of claim 26, wherein the second dose is administered 26 to 28 weeks after the first dose.

28. The method of claim 26, wherein the second dose is administered at least about 26 weeks, about 27 weeks, about 28 weeks, about 30 weeks, about 35 weeks, about 40 weeks, or about 45 weeks after the first dose.

29. The method of any one of claims 26-28, wherein the subject experiences a reduction in symptoms of depression after administering the first dose of psilocybin.

30. The method of any one of claims 26-29, further comprising measuring the subject's depressive symptoms after administering the first dose of psilocybin using a clinical depression rating scale.

31. The method of claim 30, wherein the clinical depression rating scale is MADRS.

32. The method of claim 31, wherein the subject has a change in baseline in the MADRS of −10 to −30 after administering the first dose if psilocybin.

33. The method of claim 32, wherein the subject has a change in baseline in the MADRS of −11, −12, −13, −14, −15, −16, −17, −18, −19, −20, −21, −22, −23, −24, or −25, after administering the first dose of psilocybin.

34. The method of claim 32 or 33, wherein the change in baseline is the MADRS is measured on Day 2, Day 3, Day 4, Day 5, or Day 6 after administering the first dose of psilocybin or 1 week, 5 weeks, 10 weeks, 12 weeks, 15 weeks, 16 weeks, 20 weeks, 24 weeks, 25 weeks, or 28 weeks after administering the first dose of psilocybin, or up to 1 day before administering the second dose.

35. The method of any one of claims 26-34, wherein the subject continues to experience no or substantially no increase in symptoms of depression after administration of the second dose of psilocybin.

36. The method of claim 35, wherein the subject maintains a change in baseline in the MADRS of −10 to −30 after administering the second dose.

37. The method of claim 36, wherein the subject maintains a change in baseline in the MADRS of −11, −12, −13, −14, −15, −16, −17, −18, −19, −20, −21, −22, −23, −24, −25, −26, −27, −28, −29, or −30 after administering the second dose.

38. The method of any one of claims 26-37, wherein the first dose comprises 1 mg of psilocybin and second dose comprises 1 mg of psilocybin.

39. The method of any one of claims 27-37, wherein the first dose comprises 10 mg of psilocybin and second dose comprises 10 mg of psilocybin.

40. The method of any one of claims 27-37, wherein the first dose comprises 25 mg of psilocybin and second dose comprises 25 mg of psilocybin.

41. The method of any one of claims 26-40, wherein the psilocybin is administered to the subject in a pharmaceutical composition.

42. The method of claim 41, wherein the pharmaceutical composition comprises psilocybin, pregelatinized starch, and sodium stearyl fumarate.

43. The method of claim 41 or 42, wherein the pharmaceutical composition comprises about 1%-10%, by weight, psilocybin.

44. The method of any one of claims 41-43, wherein the pharmaceutical composition comprises about 85-99%, by weight, pregelatinized starch.

45. The method of any one of claims 41-44, wherein the pharmaceutical composition comprises about 0.5%-2%, by weight, sodium stearyl fumarate.

46. The method of any one of claims 26-45, wherein the psilocybin is crystalline psilocybin characterized by XRPD peaks at 11.5±0.1, 12.0±0.1, 14.5±0.1, 17.5±0.1 and 19.7±0.1°2θ.

47. The method of claim 46, wherein the crystalline psilocybin has a chemical purity of greater than 97% as determined by HPLC analysis.

48. The method of claim 46 or 47, wherein the crystalline psilocybin has no single impurity of greater than 2%.

50. The method of claim 1, 2, or 26, wherein the first dose comprises about 1-25 mg of psilocybin.

51. The method of claim 1, 2, or 26, wherein the second dose comprises about 1-25 mg of psilocybin.

52. The method of any one of claims 26-51, wherein the subject does not experience a depressive event for at least about 27 weeks after administering the first dose of psilocybin.

53. The method of claim 52, wherein the depressive event comprise initiation of new antidepressant treatment (first new antidepressant treatment in time period only); hospitalization due to depression/suicidality; suicide attempt, prevention of an imminent suicide attempt, or completed suicide; increased suicidality measured by worsening on MADRS item 10; active suicidal ideation measured by the C-SSRS; MADRS worsening; or discontinuation for adverse event or lack of efficacy.

54. The method of any one of claims 1-25, wherein the subject does not experience a depressive event for at least about 27 weeks after administering the first second of psilocybin.

55. The method of claim 54, wherein the depressive event comprise initiation of new antidepressant treatment (first new antidepressant treatment in time period only); hospitalization due to depression/suicidality; suicide attempt, prevention of an imminent suicide attempt, or completed suicide; increased suicidality measured by worsening on MADRS item 10; active suicidal ideation measured by the C-SSRS; MADRS worsening; or discontinuation for adverse event or lack of efficacy.
